(19) United States
(12) Patent Application Publication
(10) Pub. No.: US 2021/0130312 A1
(43) Pub. Date: May 6, 2021

(54) INHIBITORS OF EYA3-PROTEIN TYROSINE PHOSPHATASE IN DNA DAMAGE REPAIR SIGNALING OF PULMONARY ARTERIAL HYPERTENSION

(71) Applicant: CHILDREN'S HOSPITAL MEDICAL CENTER, Cincinnati, OH (US)

(72) Inventor: Rashmi Hegde, Cincinnati, OH (US)

(21) Appl. No.: 17/044,714

(22) PCT Filed: Mar. 29, 2019
(86) PCT No.: PCT/US2019/024979

§ 371 Date: Oct. 1, 2020

Related U.S. Application Data
(60) Provisional application No. 62/652,154, filed on Apr. 3, 2018.

(51) Int. Cl. C07D 307/80; A61P 9/12

(52) U.S. Cl. CPC C07D 307/80; A61P 9/12

(57) ABSTRACT

Inhibitors of EYA3-protein tyrosine phosphatase are provided herein, as well as pharmaceutical compositions and methods relating thereto.

### STATEMENT REGARDING FEDERALLY SPONSORED R&D

The invention was made with government support under HL119810 awarded by the National Institutes of Health. The government has certain rights in the invention.

### BACKGROUND

### Field

The subject matter provided herein relates to inhibition of EYA3-protein tyrosine phosphatase (EYA3-PTP); in particular compounds, compositions and methods relating to inhibition of EYA3-PTP.

### Description of the Related Technology

Protein tyrosine phosphatases (PTPs) are a group of enzymes that remove phosphate groups from phosphorylated tyrosine residues on proteins. Protein tyrosine (pTyr) phosphorylation is a post-translational modification that can create recognition motifs for protein interactions and cellular localization, affect protein stability, and regulate enzyme activity. Maintaining an appropriate level of protein tyrosine phosphorylation activity plays a role in many cellular functions.

Pulmonary Arterial Hypertension (PAH) is a progressive, life-threatening disorder typically diagnosed in middle-age for which there is no cure. Treatment largely targets the symptoms and approximately 50% of patients die within 5 years of diagnosis.

PAH occurs when the pressure in the pulmonary artery rises accompanied by thickening and stiffening of the vessel walls. PAH is more common in women, is frequently diagnosed in the prime of life, and is life-threatening. The precise causes of PAH pathology are not understood, but it is hypothesized that a combination of genetic predisposition and environmental risk factors are involved in the initial stages of the disease. About 50% of familial primary pulmonary hypertension is associated with mutations in BMPR2, and the majority of others have genetic linkage to areas near the BMPR2 gene. Additional risk factors include chronic pulmonary diseases, congenital heart diseases. HIV infection, liver disease and some diet drugs. In 2-10 persons per million PAH has no known cause (idiopathic iPAH). PAH is commonly treated with phosphodiesterase inhibitors (sildenafil and Tadalafil), endothelin receptor antagonist (Bosentan), and prostacycline analogs (Illoprost and IV Epoprostanolo). However, none of these treatments is directed towards the pathological vascular remodeling and there remains no cure, underscoring the need for novel therapeutic approaches.

Pulmonary vascular remodeling (PVR) is a crucial pathological characteristic of PAH. It is driven by hyper-proliferation and apoptosis-resistance of pulmonary arterial smooth muscle cells (PASMC) leading to occlusion of distal pulmonary arteries. No current treatments for PAH are directed towards this fundamental pathology. PVR is promoted by the ability of PASMC from PAH patients to survive and proliferate in the presence of elevated levels of reactive oxygen species (ROS) and basal DNA damage, a property dependent on the upregulation of DNA Damage Repair (DDR) mechanisms.

DNA Damage, Reactive Oxygen Species (ROS), and PAH: Pulmonary vasoconstriction caused by chronic hypoxia is generally accepted as an initiating event in many lung diseases including PAH. Tissue hypoxia activates a stress response, most notably stabilization of the oxygen tension-dependent master regulator HIF-1a. HIF-1a, in turn, triggers expression of a host of pro-angiogenic, pro-proliferative and pro-inflammatory genes. Hence hypoxia and inflammation are inextricably linked. Chronic hypoxia and systemic inflammation are both strongly associated with PAH, and can cause DNA damage. There is much evidence for higher levels of DNA damage and altered DNA damage control as well as increased ROS in PAH lungs. Notably even peripheral blood cells from PAH patients have higher levels of basal DNA damage and are more susceptible to drug-induced DNA damage. Similar susceptibility to DNA damage was found in relatives of PAH patients, and did not correlate with BMPR2 mutations. Endothelial cells in PAH plexiform lesions (PAECs) show microsatellite instability and PAEC from lung explants of PAH patients have cytogenetic abnormalities. Curiously, while elevated ROS and DNA damage is also present in PAH-PASMC, there is no significant increase in chromosomal aberrations.

Development of inhibitors of protein tyrosine phosphatases may be a promising new avenue for treatment of PAH.

### SUMMARY

Provided are compounds, compositions and methods relating to the inhibition of EYA3-PTP. In some embodiments, the methods provided herein include a compound having the structure of Formula I:

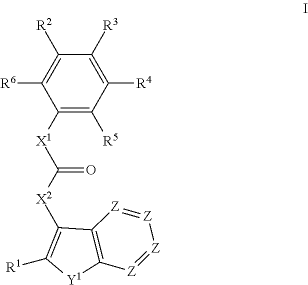

or a pharmaceutically acceptable salt thereof.

wherein:

R¹ is selected from the group consisting of C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, and amino, said C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl are each optionally substituted with one or more R^{1A};

each R^{1A} is independently selected from the group consisting of hydroxy, halo, cyano, nitro, C₁₋₆ alkyl optionally substituted with up to 5 fluoro, C₁₋₆ alkoxy optionally substituted with up to 5 fluoro;

R² is selected from the group consisting of H (hydrogen), halo, hydroxy, and C₁₋₆ alkyl substituted with one or more hydroxy;

R³ is selected from the group consisting of halo, hydroxy, and C₁₋₆ alkyl substituted with one or more hydroxy;

R⁴ is H (hydrogen) or halo;

R⁵ and R⁶ are each independently selected from the group consisting of H (hydrogen), halo, cyano, C₁₋₆ alkyl, aryl, heteroaryl, heterocyclyl, and amino, said C₁₋₆ alkyl, aryl, heteroaryl, and heterocyclyl each optionally substituted with one or more R^{1A};

X¹ is [C(R^{2A})₂]ₙ, O (oxygen), or NR^{2A}, or X¹ is absent;

X² is [C(R^{2A})₂]ₙ, O (oxygen), or NR^{2A}, or X² is absent;

each R² is independently selected from the group consisting of H (hydrogen), halo, hydroxy, O-carbamyl, N-carbamyl, C-amido, S-sulfonamido, N-sulfonamido, C-carboxy, amino, aryl, heteroaryl, heterocyclyl, arylalkyl, heteroarylalkyl, heterocyclylalkyl, cycloalkyl, cycloalkenyl, (cyclolalkyl)alkyl, C₁₋₆ alkyl substituted with one or more hydroxyl, and C₁₋₆ alkyl optionally substituted with up to 5 fluoro;

each n is independently 1 or 2;

Y¹ is O (oxygen), S (sulfur), or NR^{2A}; and

each Z is independently selected from the group consisting CR^{2A}, and N (nitrogen).

In some embodiments, the methods provided herein include a compound having the structure of Formula IV:

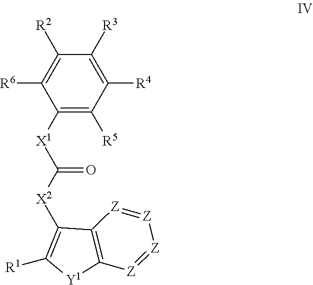

or a pharmaceutically acceptable salt thereof, wherein:

R¹ is selected from the group consisting of C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, and amino, said C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl are each optionally substituted with one or more R^{1A};

each R^{1A} is independently selected from the group consisting of hydroxy, halo, cyano, nitro, C₁₋₆ alkyl optionally substituted with up to 5 fluoro, C₁₋₆ alkoxy optionally substituted with up to 5 fluoro, O-carbamyl, N-carbamyl O-thiocarbamyl, N-thiocarbamyl, C-amido, N-amido, S-sulfonamido, N-sulfonamido, C-carboxy, O-carboxy, isocyanato, thiocyanato, isothiocyanato, nitro, and amino;

R² is selected from the group consisting of H (hydrogen), halo, hydroxy, O-carbamyl, N-carbamyl, O-thiocarbamyl, N-thiocarbamyl, C-amido, N-amido, S-sulfonamido, N-sulfonamido, C-carboxy, O-carboxy, and C₁₋₆ alkyl substituted with one or more hydroxy;

R³ is selected from the group consisting of halo, hydroxy, O-carbamyl, N-carbamyl, C-amido, S-sulfonamido, N-sulfonamido, C-carboxy, amino, and C₁₋₆ alkyl substituted with one or more hydroxy:

R⁴ is selected from the group consisting of H (hydrogen), halo, and C alkyl optionally substituted with up to 5 fluoro and C alkoxy optionally substituted with up to 5 fluoro;

R⁵ and R⁶ are each independently selected from the group consisting of H (hydrogen), halo, cyano, C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, and amino, said C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl each optionally substituted with one or more R^{1A};

X¹ is [C(R^{2A})₂]ₙ, O (oxygen), or NR^{2A}, or X¹ is absent;

X² is [C(R^{2A})₂]ₙ, O (oxygen), or NR^{2A}, or X² is absent;

each R^{2A} is independently selected from the group consisting of H (hydrogen), halo, hydroxy, O-carbamyl, N-carbamyl, C-amido, S-sulfonamido, N-sulfonamido, C-carboxy, amino, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, C₁₋₆ alkyl substituted with one or more hydroxyl, and C₁₋₆ alkyl optionally substituted with up to 5 fluoro;

each n is independently 1 or 2;

Y¹ is O (oxygen), S (sulfur), or NR^{2A}; and each Z is independently selected from the group consisting CR^{2A}, and N (nitrogen);

Also presented herein is a compound having the structure of Formula I for use in treating pulmonary arterial hypertension. Also presented herein is a compound having the structure of Formula I for use in attenuating pulmonary vascular remodeling in an individual. Also presented herein is a compound having the structure of Formula I for use in preventing, reducing, or lowering the risk of vascular smooth muscle cell hyperproliferation or preventing or reducing thickening of pulmonary artery walls in an individual.

Also presented herein is a method of treating preventing, reducing the symptoms, and lowering the risk of a disease in an individual, including; selecting or identifying an individual having or at risk of pulmonary arterial hypertension; and administering to the individual an effective amount of a compound having the structure of Formula IV. In some embodiments, the compound of Formula IV has the structure of Formula V. Also presented herein is a method of preventing, reducing, or lowering the risk of vascular smooth muscle cell hyperproliferation, or preventing or reducing thickening of pulmonary artery walls in an individual, including; selecting or identifying an individual; and administering to the individual an effective amount of a compound having the structure of Formulae I, II, III or IV. Also presented herein is a method of effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy in an individual, including; selecting or identifying an individual; and administering to the individual an effective amount of a compound having the structure of Formulae I, II, III or IV.

Also presented herein is a composition comprising a pharmaceutically acceptable excipient, and a compound having the structure of Formulae I, II, III or IV.

Also presented herein is a method for evaluating the inhibition of EYA3-PTP comprising contacting a full-length EYA3-PTP with a compound from a library of compounds and evaluating the results; wherein the compound has a user selected relative level of inhibitory activity compared to the inhibitory activity of the same compound when it contacts the catalytic domain (ED) of EYA3-PTP.

Also presented herein is a method for evaluating the inhibition of EYA3-PTP comprising: a) contacting the catalytic domain (ED) of EYA3-PTP with a compound from a library of compounds and evaluating the results; and b) contacting a full-length EYA3-PTP with the compound and evaluating the results. The method can further comprise: c) performing a) for each compound in the library of compounds; d) selecting one or more compounds from c) that inhibit the catalytic domain (ED) of EYA3-PTP according to a user-selected level; e) performing b) for each compound selected in d); and f) selecting one or more compounds from e) that inhibit full-length EYA3-PTP according to a user-selected level.

Also presented herein is a method for identifying a compound that specifically inhibits EYA3-PTP comprising: a) contacting EYA3-PTP with a compound and evaluating the results; and b) contacting a cysteine catalysis-based protein tyrosine phosphatase or an FCP/SCP family protein tyrosine phosphatase with a compound have the structure of Formulae I, II, III or IV and evaluating the results.

Also presented herein is a method of evaluating a compound for inhibition of DNA Damage Repair or pulmonary vascular remodeling, comprising contacting EYA3-PTP with a compound and evaluating the results. In some embodiments, the compound comprises a compound having the structure of Formulae I III or IV.

Also presented herein is a method of evaluating a compound for hepatotoxicity, comprising contacting immortaliaed human hepatocytes (HepG2) with a compound and evaluating the results. In some embodiments, the compound comprises a compound having the structure of Formulae I, II, III, or IV.

Also presented herein is a method of treating a disease in an individual, including; selecting or identifying an individual having angio-obliterative pulmonary hypertension, or idiopathic pulmonary arterial hypertension; and administering to the individual an effective amount of a compound having the structure of Formulae I, II, III or IV.

Also presented herein is a compound having the structure of Formulae I, II, III or IV for use in treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy. Also presented herein is a compound having the structure of Formulae I, II, III or IV for use in preventing, reducing, or lowering the risk of vascular smooth muscle cell hyperproliferation, or preventing or reducing thickening of pulmonary artery walls. Also presented herein is a compound having the structure of Formulae I, II, III or IV for use in effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy.

Also presented herein is a method of treating proliferative retinopathy, retinopathy of prematurity, diabetic retinopathy, age related macular degeneration, retinal vasculitis, exudative vitreoretinopathy, tumor angiogenesis, hemangiomas, tumor metastasis, treating breast cancer, ductal carcinoma lobule carcinoma, breast epithelial cancer, ovarian cancer, including epithelial ovarian cancer, desmoid tumor, malignant peripheral nerve sheath cancer, acute leukemia, rhabdomyosarcoma. Ewing's sarcoma, extra-skeletal myxoid chondrosarcoma, or endometrial cancer with a compound have the structure of Formulae I, II or III or IV.

### DETAILED DESCRIPTION

It is to be understood that both the foregoing general description and the following detailed description are exemplary and explanatory only and are not restrictive of the embodiments claimed. In this application, the use of the singular includes the plural unless specifically stated otherwise. In this application, the use of “or” means “and/or” unless stated otherwise. Furthermore, use of the term “including” as well as other forms, such as “includes.” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

The Eyes Absent protein (EYA3) is a protein tyrosine phosphatase (PTP) that promotes survival in the survival-versus-apoptosis decision of DNA damaged cells.

Provided are compounds, compositions and methods relating to the inhibition of EYA3-protein tyrosine phosphatase.

In some embodiments, the compounds, compositions and methods provided herein include a compound having the structure of Formula I:

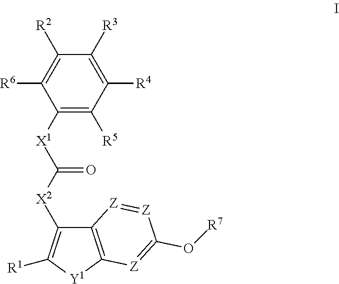

or a pharmaceutically acceptable salt thereof, wherein:
R¹ is selected from the group consisting of C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, and amino, said C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl are each optionally substituted with one or more R^{1A};
each R^{1A} is independently selected from the group consisting of hydroxy, halo, cyano, nitro, —C(═O)N(R^{1AA}))₂, C₁₋₆alkyl optionally substituted with up to 5 fluoro, and C₁₋₆ alkoxy optionally substituted with up to 5 fluoro;
each R^{1A} is independently selected from the group consisting of H (hydrogen), C₁₋₆ alkyl optionally substituted with up to 5 fluoro, and C₁₋₆ alkyl substituted with one or more hydroxyl;
R² is selected from the group consisting of H (hydrogen), halo, hydroxy, and C₁₋₆ alkyl substituted with one or more hydroxy;
R³ is selected from the group consisting of halo, hydroxy, and C₁₋₆ alkyl substituted with one or more hydroxy;
R⁴ is H (hydrogen) or halo;
R⁵ and R⁶ are each independently selected from the group consisting of H (hydrogen), halo, cyano, C₁₋₆ alkyl, aryl, heteroaryl, heterocyclyl, and amino, said C₁₋₆ alkyl, aryl, heteroaryl, and heterocyclyl each optionally substituted with one or more R^{1A};
R⁷ is selected from the group consisting of —C(═O)aryl, —C(═O)C₁₋₆ alkyl and C₁₋₆ alkyl, said C₁₋₆ alkyl optionally substituted with one or more R^{1B};
each R^{1B} is independently selected from the group consisting of hydroxy, halo, aryl, heteroaryl, C₁₋₆ alkoxy optionally substituted with up to 5 fluoro;
X¹ is [C(R^{2A})₂]ₙ, O (oxygen), or NR^{2A}, or X¹ is absent;
X² is [C(R^{2A})₂]ₙ, O (oxygen), or NR^{2A}, or X² is absent;
each R² is independently selected from the group consisting of H (hydrogen), halo, hydroxy, O-carbamyl, N-carbamyl, C-amido, S-sulfonamido. N-sulfonamido, C-carboxy, amino, aryl, heteroaryl, heterocyclyl, arylalkyl, heteroarylalkyl, heterocyclylalkyl, cycloalkyl, cycloalkenyl, (cyclolalkyl)alkyl, C₁₋₆ alkyl substituted with one or more hydroxyl, and C₁₋₆ alkyl optionally substituted with up to 5 fluoro;
each n is independently 1 or 2;
Y¹ is O (oxygen), S (sulfur), or NR^{2A}; and

each Z is independently selected from the group consisting CR^{2A}, and N (nitrogen).

In some embodiments, R⁵ and R⁶ are H (hydrogen). In some embodiments, R² is iodo or bromo. In some embodiments, R⁴ is iodo or bromo.

In some embodiments, the compound having the structure of Formula I may have the structure of Formula Ia, or Ib,

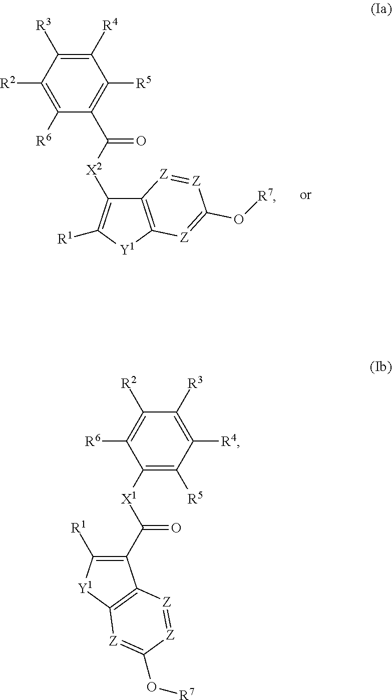

or a pharmaceutically acceptable salt thereof.

In some embodiments, R¹ is C₁₋₆ alkyl optionally substituted with one or more R^{1A}. In some embodiments, X¹ is [C(R^{2A})₂], or NR^{2A}. In some embodiments, X² is [C(R^{2A})₂], or NR^{2A}. In some embodiments, Y¹ is O (oxygen), or S (sulfur). In some embodiments, each Z is CR^{2A}, where each R^{2A} is independently selected from the group consisting of H (hydrogen) and hydroxy. In some embodiments, X¹ or X² is NR^{2A}. In some embodiments, X² is NR^{2A}.

In some embodiments, each Z is CH.

In some embodiments, the compound having the structure of Formula I may have the structure of Formula II:

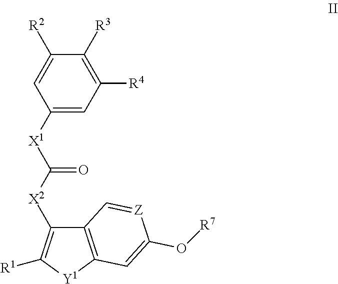

or a pharmaceutically acceptable salt thereof, wherein:

X¹ is O (oxygen), or NR^{2A}, or X¹ is absent;

X² is O (oxygen), or NR^{2A}, or X² is absent;

each R² is independently selected from the group consisting of H (hydrogen), halo, hydroxy, C₁₋₆ alkyl substituted with one or more hydroxyl, and C₁₋₆ alkyl optionally substituted with up to 5 fluoro; and

Y¹ is O (oxygen), or S (sulfur).

In some embodiments, the compound having the structure of Formula II may have the structure of Formula IIa, or IIb,

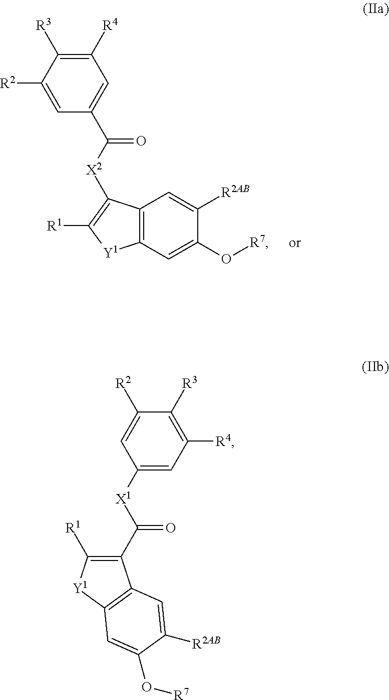

or a pharmaceutically acceptable salt thereof, wherein R² and R⁴ are each independently H (hydrogen) or halo; and each R^{2A} is independently hydrogen, halo or hydroxyl, wherein at least one R^{2A} is hydroxyl.

In some embodiments, R¹ is C₁₋₆ alkyl optionally substituted with one or more hydroxy. In some embodiments, X¹ is or NR^{2A}. In some embodiments, X² is or NR^{2A}.

In some embodiments, the compound of Formula I may have the structure of Formula III:

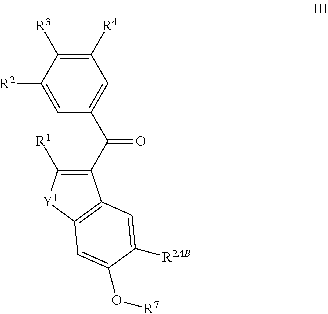

or a pharmaceutically acceptable salt thereof, wherein:

R³ is halo or hydroxy;

R^{2AA} is H (hydrogen) or hydroxyl;

R^{2AB} is H (hydrogen) or hydroxyl; and Y¹ is O (oxygen), or S (sulfur). In some embodiments, R^{2AA} is hydroxyl. In some embodiments, R^{2AB} is hydroxyl). In some embodiments, R^{2AA} is H (hydrogen). In some embodiments, R^{2AB} is H (hydrogen).

In some embodiments, Y¹ is O (oxygen). In some embodiments, R¹ is selected from the group consisting of C₁₋₆ alkyl, cycloalkyl, (cyclolalkyl)alkyl, each optionally substituted with one or more hydroxy. In some embodiments, R² is selected from the group consisting of halo, hydroxy, O-carbamyl, N-carbamyl, C-amido, N-amido, S-sulfonamido, N-sulfonamido, C-carboxy, O-carboxy, and C₁₋₃ alkyl substituted with one or more hydroxy. In some embodiments, R³ is selected from the group consisting of hydroxy and C₁₋₃ alkyl substituted with one or more hydroxy. In some embodiments, R⁴ is compound from the group consisting of halo, C₁₋₃ alkyl optionally substituted with up to 5 fluoro, and C₁₋₃ alkoxy optionally substituted with up to 5 fluoro.

In some embodiments, each R^{2A} is independently selected from the group consisting of H (hydrogen), and C₁₋₆ alkyl optionally substituted with up to 5 fluoro. In some embodiments, each R^{2AA} is H (hydrogen). In some embodiments, R¹ is selected from the group consisting of C₁₋₃ alkyl, C-amido, and S-sulfonamido, said C₁₋₃ alkyl optionally substituted with one or more hydroxy. In some embodiments, R² is selected from the group consisting of hydroxy, C-amido, N-amido, S-sulfonamido, and C₁₋₃ alkyl substituted with hydroxy. In some embodiments, R³ is selected from the group consisting of hydroxy, C-amido, and C₁₋₃ alkyl substituted with hydroxy. In some embodiments, R⁴ is selected from the group consisting of fluoro, chloro, bromo, methyl, —CF₃, —OCH₃, and —OCF₃. In some embodiments, R² is selected from the group consisting of fluoro, chloro, bromo, and iodo. In some embodiments, R⁴ is selected from the group consisting of fluoro, chloro, bromo, and iodo. In some embodiments, R¹ is substituted C₁₋₆ alkyl, and R^{1A} is hydroxy. In some embodiments, R¹ is C₁₋₆ alkyl optionally substituted with one or more R^{1A}. In some embodiments, R¹ is ethyl. In some embodiments, R² is iodo or bromo. In some embodiments, R⁴ is iodo or bromo.

In some embodiments, the compounds, compositions and methods provided herein include a compound having the structure of Formula IV

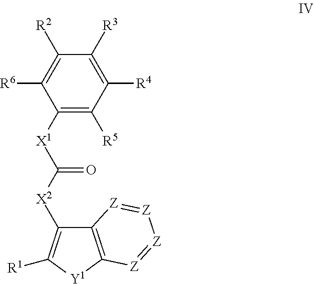

or a pharmaceutically acceptable salt thereof, wherein:

R¹ is selected from the group consisting of C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, and amino, said C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl are each optionally substituted with one or more R^{1A};

each R^{1A} is independently selected from the group consisting of hydroxy, halo, cyano, nitro, C₁₋₆ alkyl optionally substituted with up to 5 fluoro, C₁₋₆ alkoxy optionally substituted with up to 5 fluoro, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, and amino;

R² is selected from the group consisting of H (hydrogen), halo, hydroxy, O-carbamyl, N-carbamyl, O-thiocarbamyl, N-thiocarbamyl, C-amido, N-amido, S-sulfonamido, N-sulfonamido, C-carboxy, O-carboxy, and C₁₋₆ alkyl substituted with one or more hydroxy;

R³ is selected from the group consisting of halo, hydroxy, O-carbamyl, N-carbamyl, C-amido, S-sulfonamido, N-sulfonamido, C-carboxy, amino, and C₁₋₆ alkyl substituted with one or more hydroxy:

R⁴ is selected from the group consisting of H (hydrogen), halo, and C₁₋₆ alkyl optionally substituted with up to 5 fluoro and C₁-6 alkoxy optionally substituted with up to 5 fluoro;

R⁵ and R⁶ are each independently selected from the group consisting of H (hydrogen), halo, cyano, C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, O-carbamyl, N-carbamyl, O-thiocarbamyl, N-thiocarbamyl, C-amido, N-amido, S-sulfonamido. N-sulfonamido, C-carboxy, O-carboxy, isocyanato, thiocyanato, isothiocyanato, nitro, and amino, said C₁₋₆ alkyl, C₂₋₆ alkenyl. C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl each optionally substituted with one or more R^{1A};

X¹ is [C(R^{2A})₂]ₙ, O (oxygen), or NR^{2A}, or X¹ is absent;

X² is [C(R^{2A})₂]ₙ, O (oxygen), or NR^{2A}, or X² is absent;

each R² is independently selected from the group consisting of H (hydrogen), halo, hydroxy, O-carbamyl, N-carbamyl, C-amido, S-sulfonamido, N-sulfonamido, C-carboxy, amino, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, C₁₋₆ alkyl substituted with one or more hydroxyl, and C₁₋₆ alkyl optionally substituted with up to 5 fluoro;

each n is independently 1 or 2;

Y¹ is O (oxygen). S (sulfur), or NR^{2A}; and each Z is independently selected from the group consisting CR^{2A}, and N (nitrogen).

In certain embodiments, a compound, composition or method as disclosed herein is provided, wherein the compound having the structure of Formula I has the structure of Formula Ia, or Ib,

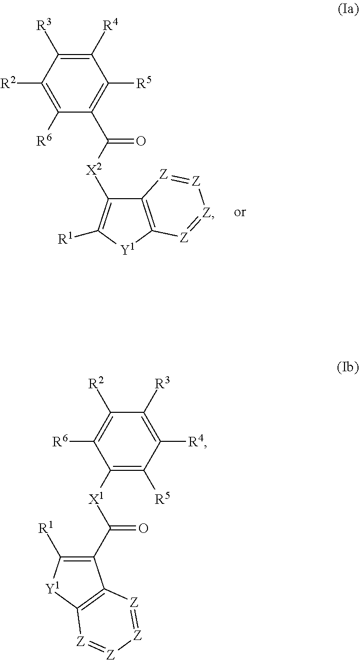

or a pharmaceutically acceptable salt thereof.

In certain embodiments, a compound, composition or method as disclosed herein is provided, wherein the compound having the structure of Formula I has the structure of Formula II:

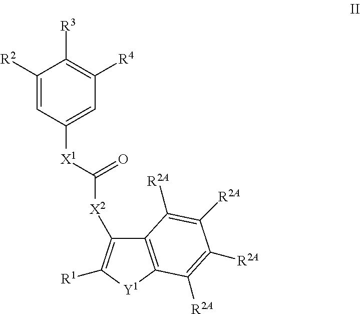

or a pharmaceutically acceptable salt thereof, wherein:

R¹ is selected from the group consisting of C₁₋₆ alkyl, C₂₋₆ alkenyl, C₂₋₆ alkynyl, aryl, arylalkyl, cycloalkyl, cycloalkenyl. (cyclolalkyl)alkyl, C-amido. N-amido, S-sulfonamido, and N-sulfonamido, said C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl are each optionally substituted with one or more R^{1A};

X¹ is O (oxygen), or NR^{2A}, or X¹ is absent:

X² is O (oxygen), or NR^{2A}, or X² is absent;

each R^{2A} is independently selected from the group consisting of H (hydrogen), halo, hydroxy. O-carbamyl, N-carbamyl, C-amido, S-sulfonamido, N-sulfonamido, C-carboxy, amino, C₂₋₆ alkenyl, C₂₋₆ alkynyl, aryl, arylalkyl, cycloalkyl, cycloalkenyl, (cyclolalkyl)alkyl, C₁₋₆ alkyl substituted with one or more hydroxyl, and C₁₋₆ alkyl optionally substituted with up to 5 fluoro; and Y¹ is O (oxygen), or S (sulfur).

In certain embodiments, a compound, composition or method as disclosed herein is provided, wherein the compound having the structure of Formula II has the structure of Formula IIa, or IIb:

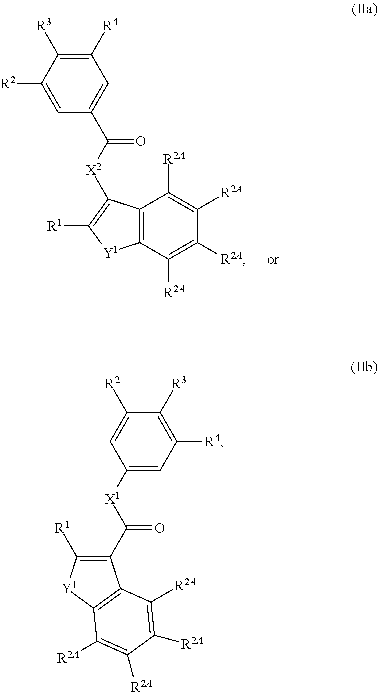

or a pharmaceutically acceptable salt thereof.

In certain embodiments, a compound, composition or method as disclosed herein is provided, wherein the compound having the structure of Formula I has the structure of Formula III:

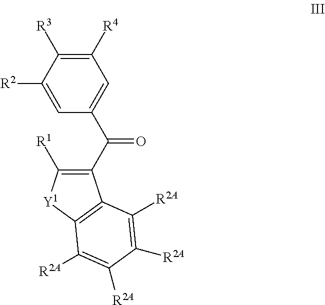

or a pharmaceutically acceptable salt thereof, wherein:

R¹ is selected from the group consisting of C₁₋₆ alkyl, C₂₋₆ alkenyl, C₂₋₆ alkynyl, cycloalkyl, cycloalkenyl, (cyclolalkyl)alkyl, C-amido, N-amido, S-sulfonamido, N-sulfonamido, said C₁₋₆ alkyl, C₂₋₆ alkenyl, C₂₋₆ alkynyl, cycloalkyl, cycloalkenyl, (cyclolalkyl)alkyl are each optionally substituted with one or more R^{1A};

each R^{1A} is independently selected from the group consisting of hydroxy, halo, C₁₋₆ alkyl substituted with up to 5 fluoro, and C₁₋₆ alkoxy optionally substituted with up to 5 fluoro;

each R^{2A} is independently selected from the group consisting of H (hydrogen), halo, hydroxy, O-carbamyl, N-carbamyl, C-amido, S-sulfonamido, N-sulfonamido, C-carboxy, amino, C₂₋₆ alkenyl, C₂₋₆ alkynyl, aryl, arylalkyl, cycloalkyl, cycloalkenyl, (cyclolalkyl)alkyl, C₁₋₆ alkyl substituted with one or more hydroxyl, and C₁₋₆ alkyl optionally substituted with up to 5 fluoro; and Y¹ is O (oxygen), or S (sulfur).

In certain embodiments, a compound, composition or method as disclosed herein is provided wherein R⁵ and R⁶ are H (hydrogen).

In certain embodiments, a compound, composition or method as disclosed herein is provided wherein R¹ is selected from the group consisting of C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, O-carbamyl, N-carbamyl, O-thiocarbamyl, N-thiocarbamyl, C-amido, S-sulfonamido, and N-sulfonamido, said C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl are each optionally substituted with one or more R^{1A}. In certain embodiments, a compound, composition or method as disclosed herein is provided wherein R¹ is C₁₋₆ alkyl. In certain embodiments, a compound, composition or method as disclosed herein is provided wherein R¹ is substituted C₁₋₆ alkyl, and R^{1A} is hydroxy.

In certain embodiments, a compound, composition or method as disclosed herein is provided wherein X¹ is [C(R^{2A})₂]ₙ or NR^{2A}.

In certain embodiments, a compound, composition or method as disclosed herein is provided wherein X² is [C(R^{2A})₂]ₙ or NR^{2A}.

In certain embodiments, a compound, composition or method as disclosed herein is provided wherein Y¹ is O (oxygen), or S (sulfur).

In certain embodiments, a compound, composition or method as disclosed herein is provided wherein each Z is CR^{2A}, where each R^{2A} is independently selected from the group consisting of H (hydrogen), hydroxy, and C₁₋₆ alkyl optionally substituted with up to 5 fluoro.

In certain embodiments, a compound, composition or method as disclosed herein is provided wherein R¹ is selected from the group consisting of C₁₋₃ alkyl, C-amido, and S-sulfonamido, said C₁₋₃ alkyl optionally substituted with one or more hydroxy. In certain embodiments, a compound, composition or method as disclosed herein is provided wherein R¹ is C₁₋₆ alkyl. In certain embodiments, a compound, composition or method as disclosed herein is provided wherein R¹ is substituted C₁₋₆ alkyl, and R^{1A} is hydroxy.

In certain embodiments, a compound, composition or method as disclosed herein is provided wherein R² is selected from the group consisting of hydroxy. C-amido, N-amido, S-sulfonamido, and C₁₋₃ alkyl substituted with hydroxy. In certain embodiments, a compound, composition or method as disclosed herein is provided wherein R² is bromo. In certain embodiments, a compound, composition or method as disclosed herein is provided wherein R² is iodo.

In certain embodiments, a compound, composition or method as disclosed herein is provided wherein R³ is selected from the group consisting of hydroxy, C-amido, and C₁₋₃ alkyl substituted with hydroxy.

In certain embodiments, a compound, composition or method as disclosed herein is provided wherein each Z is CH.

In certain embodiments, a compound, composition or method as disclosed herein is provided wherein R¹ is selected from the group consisting of C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, each optionally substituted with one or more R^{1A}. In certain embodiments, a compound, composition or method as disclosed herein is provided wherein R¹ is C₁₋₆ alkyl. In certain embodiments, a compound, composition or method as disclosed herein is provided wherein R¹ is substituted C₁₋₆ alkyl, and R^{1A} is hydroxy.

In certain embodiments, a compound, composition or method as disclosed herein is provided wherein X¹ is or NR^{2A}.

In certain embodiments, a compound, composition or method as disclosed herein is provided wherein X² is or NR^{2A}.

In certain embodiments, a compound, composition or method as disclosed herein is provided wherein Y¹ is O (oxygen).

In certain embodiments, a compound, composition or method as disclosed herein is provided wherein R¹ is selected from the group consisting of C₁₋₆ alkyl, cycloalkyl. (cyclolalkyl)alkyl, each optionally substituted with one or more R^{1A}.

In certain embodiments, a compound, composition or method as disclosed herein is provided wherein R² is selected from the group consisting of halo, hydroxy, O-carbamyl, N-carbamyl, C-amido, N-amido, S-sulfonamido, N-sulfonamido, C-carboxy, O-carboxy, and C₁₋₃ alkyl substituted with one or more hydroxy. In certain embodiments, a compound, composition or method as disclosed herein is provided wherein R² is bromo. In certain embodiments, a compound, composition or method as disclosed herein is provided wherein R² is iodo.

In certain embodiments, a compound, composition or method as disclosed herein is provided wherein R³ is selected from the group consisting of hydroxy, C-amido, S-sulfonamido, N-sulfonamido, and C₁₋₃ alkyl substituted with one or more hydroxy.

In certain embodiments, a compound, composition or method as disclosed herein is provided wherein R¹ is selected from the group consisting of halo, C₁₋₃ alkyl optionally substituted with up to 5 fluoro, and C₁₋₃ alkoxy optionally substituted with up to 5 fluoro. In certain embodiments, a compound, composition or method as disclosed herein is provided wherein R⁴ is bromo. In certain embodiments, a compound, composition or method as disclosed herein is provided wherein R⁴ is iodo.

In certain embodiments, a compound, composition or method as disclosed herein is provided wherein each R^{2A} is independently selected from the group consisting of H (hydrogen), hydroxy, and C₁₋₆ alkyl optionally substituted with up to 5 fluoro.

In certain embodiments, a compound, composition or method as disclosed herein is provided wherein each R^{2A} is H (hydrogen).

In certain embodiments, a compound, composition or method as disclosed herein is provided wherein R¹ is selected from the group consisting of C₁₋₃ alkyl, C-amido, and S-sulfonamido, said C₁₋₃ alkyl optionally substituted with one or more hydroxy.

In certain embodiments, a compound, composition or method as disclosed herein is provided wherein R² is selected from the group consisting of hydroxy. C-amido, N-amido, S-sulfonamido, and C₁₋₃ alkyl substituted with hydroxy.

In certain embodiments, a compound, composition or method as disclosed herein is provided wherein R³ is selected from the group consisting of hydroxy. C-amido, and C₁₋₃ alkyl substituted with hydroxy.

In certain embodiments, a compound, composition or method as disclosed herein is provided wherein R⁴ is selected from the group consisting of fluoro, chloro, bromo, methyl, —CF₃, —OCH₃, and —OCF₃.

In certain embodiments, a method of treating pulmonary arterial hypertension is provided, wherein the compound is:

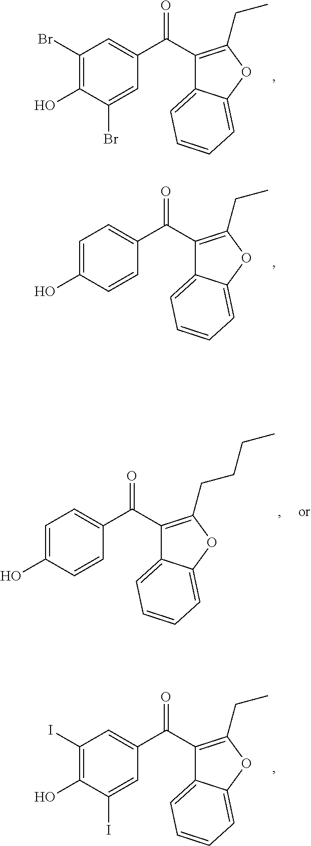 [SMILES: CCc1oc2ccccc2c1C(=O)c1cc(Br)c(O)c(Br)c1 ; CCc1oc2ccccc2c1C(=O)c1ccc(O)cc1 ; CCCCc1oc2ccccc2c1C(=O)c1ccc(O)cc1 ; CCc1oc2ccccc2c1C(=O)c1cc(I)c(O)c(I)c1]

or a pharmaceutically acceptable salt thereof.

In certain embodiments, a method of treating pulmonary arterial hypertension is provided, wherein the compound is:

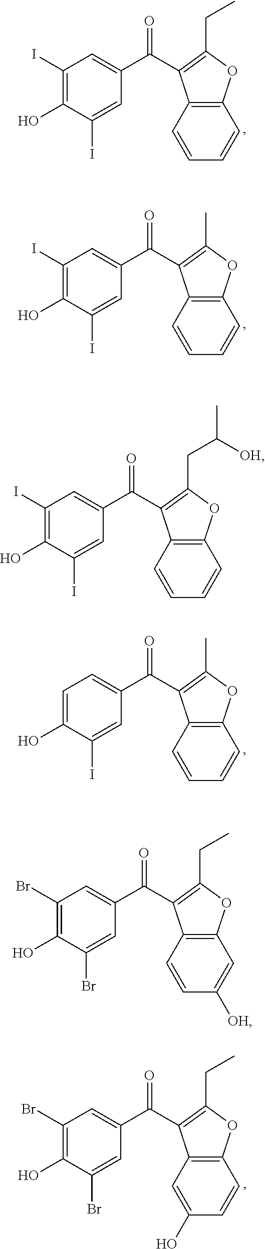

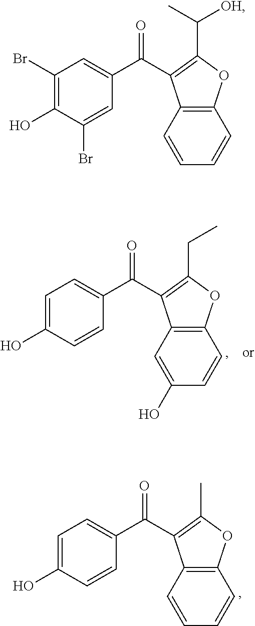 [SMILES: CC(O)c1oc2ccccc2c1C(=O)c1cc(Br)c(O)c(Br)c1 ; CCc1oc2ccc(O)cc2c1C(=O)c1ccc(O)cc1 ; Cc1oc2ccccc2c1C(=O)c1ccc(O)cc1]

or a pharmaceutically acceptable salt thereof.

In certain embodiments, a method of treating pulmonary arterial hypertension is provided, wherein the compound is:

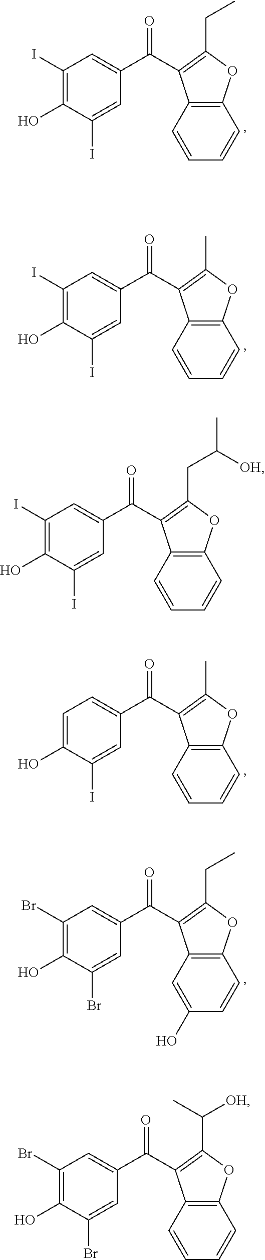

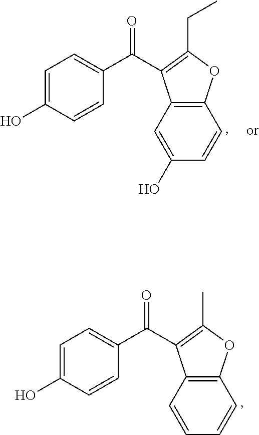 [SMILES: CCc1oc2ccc(O)cc2c1C(=O)c1ccc(O)cc1 ; Cc1oc2ccccc2c1C(=O)c1ccc(O)cc1]

or a pharmaceutically acceptable salt thereof.

In certain embodiments, a method of treating pulmonary arterial hypertension is provided, wherein the compound is:

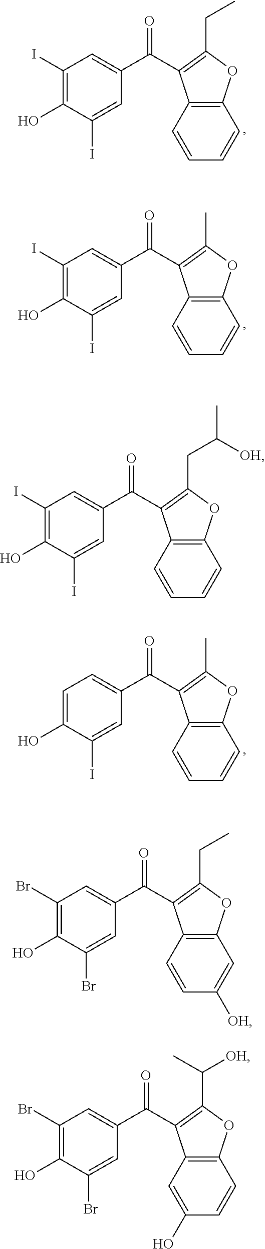

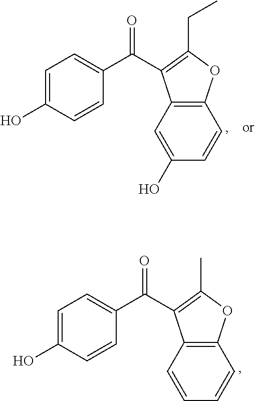 [SMILES: CCc1oc2ccc(O)cc2c1C(=O)c1ccc(O)cc1 ; Cc1oc2ccccc2c1C(=O)c1ccc(O)cc1]

or a pharmaceutically acceptable salt thereof.

In certain embodiments, a method of treating pulmonary arterial hypertension is provided, wherein the compound is:

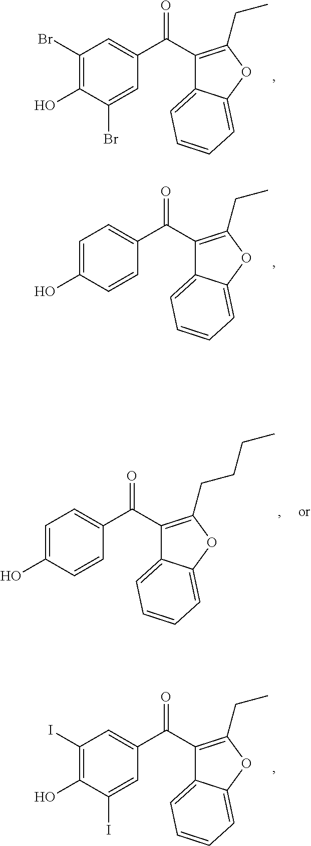 [SMILES: CCc1oc2ccccc2c1C(=O)c1cc(Br)c(O)c(Br)c1 ; CCc1oc2ccccc2c1C(=O)c1ccc(O)cc1 ; CCCCc1oc2ccccc2c1C(=O)c1ccc(O)cc1 ; CCc1oc2ccccc2c1C(=O)c1cc(I)c(O)c(I)c1]

or a pharmaceutically acceptable salt thereof.

In certain embodiments, a method of treating pulmonary arterial hypertension is provided, wherein the compound is:

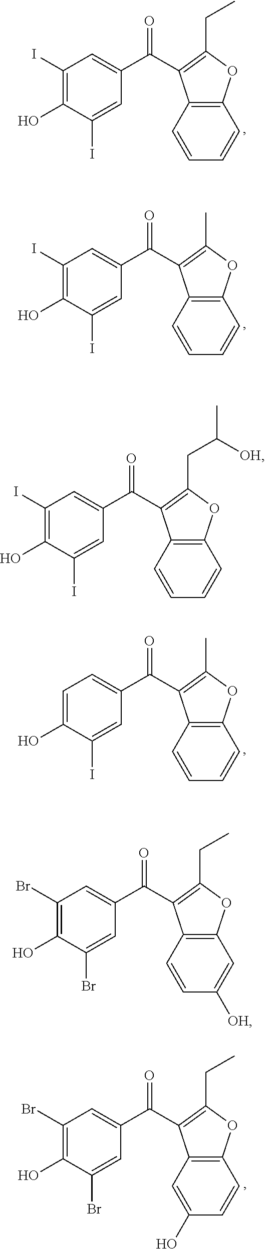

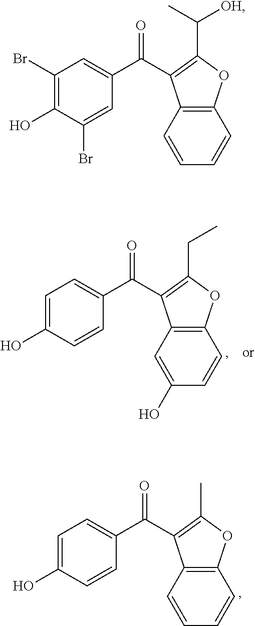 [SMILES: CC(O)c1oc2ccccc2c1C(=O)c1cc(Br)c(O)c(Br)c1 ; CCc1oc2ccc(O)cc2c1C(=O)c1ccc(O)cc1 ; Cc1oc2ccccc2c1C(=O)c1ccc(O)cc1]

or a pharmaceutically acceptable salt thereof.

In certain embodiments, a method of treating pulmonary arterial hypertension is provided, wherein the compound is:

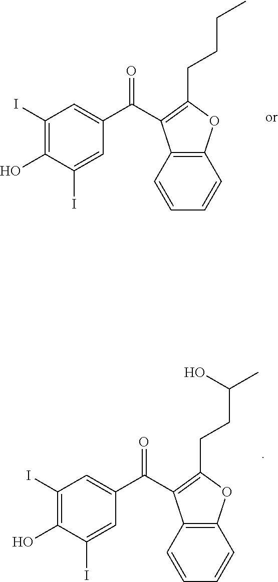 [SMILES: CCCCc1oc2ccccc2c1C(=O)c1cc(I)c(O)c(I)c1 ; CC(O)CCc1oc2ccccc2c1C(=O)c1cc(I)c(O)c(I)c1]

In certain embodiments, a method of treating pulmonary arterial hypertension is provided, wherein the compound is:

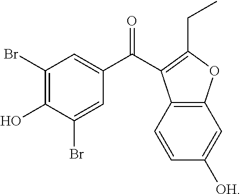 [SMILES: CCc1oc2cc(O)ccc2c1C(=O)c1cc(Br)c(O)c(Br)c1]

In certain embodiments, a compound, composition or method as disclosed herein is provided wherein R¹ is selected from the group consisting of C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, O-carbamyl, N-carbamyl, O-thiocarbamyl, N-thiocarbamyl, C-amido, N-amido, S-sulfonamido. N-sulfonamido, C-carboxy, O-carboxy, isocyanato, thiocyanato, isothiocyanato, nitro, and amino, said C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl are each optionally substituted with one or more R^{1A}; each R^{1A} is independently selected from the group consisting of hydroxy, halo, cyano, nitro, C₁₋₆ alkyl optionally substituted with up to 5 fluoro, C₁₋₆ alkoxy optionally substituted with up to 5 fluoro, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, and amino; R² is selected from the group consisting of H (hydrogen), halo, hydroxy, O-carbamyl, N-carbamyl, O-thiocarbamyl, N-thiocarbamyl, C-amido, N-amido, S-sulfonamido, N-sulfonamido, C-carboxy, O-carboxy, and C₁₋₆ alkyl substituted with one or more hydroxy; R³ is selected from the group consisting of halo, hydroxy, O-carbamyl, N-carbamyl, C-amido, S-sulfonamido, N-sulfonamido, C-carboxy, amino, and C₁₋₆ alkyl substituted with one or more hydroxy; R is selected from the group consisting of H (hydrogen), halo, and C₁₋₆ alkyl optionally substituted with up to 5 fluoro and C₁₋₆ alkoxy optionally substituted with up to 5 fluoro; R⁵ and R⁶ are each independently selected from the group consisting of H (hydrogen), halo, cyano, C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, and amino, said C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl each optionally substituted with one or more R^{1A}; X¹ is [C(R^{2A})₂]ₙ, O (oxygen), or NR^{2A}, or X¹ is absent; X² is [C(R^{2A})₂]ₙ, O (oxygen), or NR^{2A}, or X² is absent; each R^{2A} is independently selected from the group consisting of H (hydrogen), halo, hydroxy, O-carbamyl, N-carbamyl, C-amido, S-sulfonamido, N-sulfonamido, C-carboxy, amino, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, C₁₋₆ alkyl substituted with one or more hydroxyl, and C₁₋₆ alkyl optionally substituted with up to 5 fluoro; each n is independently 1 or 2; Y¹ is O (oxygen), S (sulfur), or NR^{2A}; and each Z is independently selected from the group consisting CR^{2A}, and N (nitrogen).

In certain embodiments, a compound, composition or method as disclosed herein is provided wherein R¹ is selected from the group consisting of C₁₋₆alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, O-carbamyl, N-carbamyl, O-thiocarbamyl, N-thiocarbamyl, C-amido, S-sulfonamido, and N-sulfonamido, said C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl are each optionally substituted with one or more R^{1A}.

In certain embodiments, a compound, composition or method as disclosed herein is provided wherein X¹ is [C(R^{1A})₂]ₙ or NR².

In certain embodiments, a compound, composition or method as disclosed herein is provided wherein X² is [C(R^{2A})₂]ₙ or NR^{2A}.

In certain embodiments, a compound, composition or method as disclosed herein is provided wherein Y¹ is O (oxygen), or S (sulfur).

In certain embodiments, a compound, composition or method as disclosed herein is provided wherein each Z is CR^{2A}, where each R^{2A} is independently selected from the group consisting of H (hydrogen), hydroxy, and C₁₋₆ alkyl optionally substituted with up to 5 fluoro.

In certain embodiments, a compound, composition or method as disclosed herein is provided wherein X¹ is NR^{2A}.

In certain embodiments, a compound, composition or method as disclosed herein is provided wherein X² is NR^{2A}.

In certain embodiments, a compound, composition or method as disclosed herein is provided wherein each Z is CH.

In certain embodiments, a compound, composition or method as disclosed herein is provided wherein X¹ is O (oxygen), or NR^{2A}, or X¹ is absent; X² is O (oxygen), or NR^{2A}, or X² is absent; each R^{2A} is independently selected from the group consisting of H (hydrogen), halo, hydroxy, O-carbamyl, N-carbamyl, C-amido, S-sulfonamido. N-sulfonamido, C-carboxy, amino, C₂₋₆ alkenyl, C₂₋₆ alkynyl, aryl, arylalkyl, cycloalkyl, cycloalkenyl, (cyclolalkyl)alkyl, C₁₋₆ alkyl substituted with one or more hydroxyl, and C₁₋₆ alkyl optionally substituted with up to 5 fluoro; and Y¹ is O (oxygen), or S (sulfur).

In certain embodiments, a compound, composition or method as disclosed herein is provided wherein R¹ is selected from the group consisting of C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, (cyclolalkyl)alkyl, each optionally substituted with one or more R^{1A}.

In certain embodiments, a compound, composition or method as disclosed herein is provided wherein R¹ is selected from the group consisting of C₁₋₆ alkyl, C₂₋₆ alkenyl. C₂₋₆ alkynyl, cycloalkyl, cycloalkenyl, (cyclolalkyl)alkyl, C-amido, N-amido, S-sulfonamido, N-sulfonamido, said C₁₋₆ alkyl, C₂₋₆ alkenyl, C₂₋₆ alkynyl, cycloalkyl, cycloalkenyl, (cyclolalkyl)alkyl are each optionally substituted with one or more R^{1A}; each R^{1A} is independently selected from the group consisting of hydroxy, halo, C₁₋₆ alkyl substituted with up to 5 fluoro, and C₁₋₆ alkoxy optionally substituted with up to 5 fluoro; R³ is selected from the group consisting of halo, hydroxy, O-carbamyl, N-carbamyl, C-amido, S-sulfonamido, N-sulfonamido, C-carboxy, and C₁₋₆ alkyl substituted with one or more hydroxy; each R^{2A} is independently selected from the group consisting of H (hydrogen), halo, hydroxy, O-carbamyl, N-carbamyl, C-amido, S-sulfonamido, N-sulfonamido, C-carboxy, amino, C₂₋₆ alkenyl, C₂₋₆ alkynyl, aryl, arylalkyl, cycloalkyl, cycloalkenyl, (cyclolalkyl)alkyl, C₁₋₆ alkyl substituted with one or more hydroxyl, and C₁₋₆ alkyl optionally substituted with up to 5 fluoro; and Y¹ is O (oxygen), or S (sulfur).

In certain embodiments, a compound, composition or method as disclosed herein is provided wherein R¹ is selected from the group consisting of C₁₋₆ alkyl, cycloalkyl. (cyclolalkyl)alkyl, each optionally substituted with one or more R^{1A}.

In certain embodiments, a compound, composition or method as disclosed herein is provided wherein R² is selected from the group consisting of halo, hydroxy, O-carbamyl, N-carbamyl, C-amido, N-amido, S-sulfonamido, N-sulfonamido, C-carboxy. O-carboxy, and C₁₋₃ alkyl substituted with one or more hydroxy.

In certain embodiments, a compound, composition or method as disclosed herein is provided wherein R³ is selected from the group consisting of hydroxy, C-amido, S-sulfonamido, N-sulfonamido, and C₁₋₃ alkyl substituted with one or more hydroxy.

In certain embodiments, a compound, composition or method as disclosed herein is provided wherein R¹ is compound from the group consisting of halo, C₁₋₃ alkyl optionally substituted with up to 5 fluoro, and C₁₋₃ alkoxy optionally substituted with up to 5 fluoro.

In certain embodiments, a compound, composition or method as disclosed herein is provided wherein each R² is independently selected from the group consisting of H (hydrogen), hydroxy, and C₁₋₆ alkyl optionally substituted with up to 5 fluoro.

In certain embodiments, a compound, composition or method as disclosed herein is provided wherein each R¹ is H (hydrogen).

In certain embodiments, a compound, composition or method as disclosed herein is provided wherein R¹ is selected from the group consisting of C₁₋₃ alkyl, C-amido, and S-sulfonamido, said C₁₋₃ alkyl optionally substituted with one or more hydroxy.

In certain embodiments, a compound, composition or method as disclosed herein is provided wherein R² is selected from the group consisting of hydroxy, C-amido. N-amido, S-sulfonamido, and C₁₋₃ alkyl substituted with hydroxy.

In certain embodiments, a compound, composition or method as disclosed herein is provided wherein R³ is selected from the group consisting of hydroxy, C-amido, and C₁₋₃ alkyl substituted with hydroxy.

In certain embodiments, a compound, composition or method as disclosed herein is provided wherein R¹ is selected from the group consisting of fluoro, chloro, bromo, methyl, —CF₃, —OCH₃, and —OCF₃. In certain embodiments, a compound, composition or method as disclosed herein is provided wherein R² is bromo and R⁴ is bromo. In certain embodiments, a compound, composition or method as disclosed herein is provided wherein R² is iodo and R⁴ is iodo.

Some embodiments are directed to a a compound having the structure of Formulae I, II or III for use in preventing, reducing, lowering the risk of, or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy. Also presented herein is a compound having the structure of Formulae I, II or III for use in preventing, reducing, or lowering the risk of vascular smooth muscle cell hyperproliferation, or preventing or reducing thickening of pulmonary artery walls.

Some embodiments are directed to a method of treatment of an Eya-related disorder comprising administering to an individual an effective amount of a compound having the structure of Formulae I, II or III. One pathway implicated in proliferative disorders such as cancer is the evolutionally conserved gene network termed the retinal determination gene network, or “RDGN.” Indeed, the Six and Eya families of genes, members of the RDGN, are frequently found upregulated in cancers. The Eya protein has been shown to be a protein tyrosine phosphatase (PTP). PTPs in general are emerging as important new targets for cancer therapy. Anti-vascular therapy has emerged as an extremely promising option for the treatment of several major diseases including solid tumors and hematological cancers, and the vision-compromising ailments, such as diabetic retinopathy, age-related macular degeneration (AMD) and retinopathy of prematurity (ROP). Also presented herein is a compound having the structure of Formulae I, II or III for use in treating proliferative retinopathy, retinopathy of prematurity, diabetic retinopathy, age related macular degeneration, retinal vasculitis, exudative vitreoretinopathy, tumor angiogenesis, hemangiomas, tumor metastasis, treating breast cancer, ductal carcinoma lobule carcinoma, breast epithelial cancer, ovarian cancer, including epithelial ovarian cancer, desmoid tumor, malignant peripheral nerve sheath cancer, acute leukemia, rhabdomyosarcoma, Ewing's sarcoma, extra-skeletal myxoid chondrosarcoma, or endometrial cancer in an individual. Also presented herein is a compound having the structure of Formulae I, II or III for use in treating breast cancer, ductal carcinoma lobule carcinoma, breast epithelial cancer, ovarian cancer, including epithelial ovarian cancer, desmoid tumor, malignant peripheral nerve sheath cancer, acute leukemia, rhabdomyosarcoma, Ewing's sarcoma, extra-skeletal myxoid chondrosarcoma, or endometrial cancer in an individual.

In view of the data provided herein, the protein tyrosine phosphatases of the Eyes Absent family (Eyes Absent phosphatases or EYA-PTPs) are highly likely to be useful drug targets in anti-vascular therapy. Also, in view of the data provided herein. Eyes Absent phosphatases are highly likely to be useful drug targets in preventing, reducing, lowering the risk of, or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy. Eyes Absent phosphatases are expressed in vascular smooth muscle cells. Also, Eyes Absent phosphatases are expressed in vascular endothelial cells (VECs) and the phosphatase activity enhances cell migration and the formation of vessel-like structures in culture. Agents that specifically target PTPs have enormous potential in the treatment of PAH, and proliferative, invasive and/or metastatic, angiogenic and/or vascular disorders such as cancer, given the significant increase in PTP activity in many disease states. Though approximately 30% of cellular proteins are phospho-proteins, tyrosine phosphorylation accounts for only about 0.01% to about 0.05% of all phospho-proteins. In disease states such as oncogenic transformation, however, tyrosine phosphorylation is increased up to one to two hundred-fold to 1 to 2% of the total phospho-protein population. While protein tyrosine phosphatases have been extensively linked with disease states including proliferative diseases such as cancer, effective tyrosine phosphatase inhibitors have traditionally been confounded by a lack of specificity, and there remains a significant need in identifying PTP specific inhibitors for the treatment of disorders involving PTP dysregulation.

Accordingly, in some embodiments, methods are provided for the treatment of proliferative retinopathy, retinopathy of prematurity, diabetic retinopathy, age related macular degeneration, retinal vasculitis, or exudative vitreoretinopathy using a compound of having the structure of Formulae I, II or III.

In some embodiments, methods are provided for treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy using a compound as disclosed and described herein. In some embodiments, methods are provided for preventing, reducing, or lowering the risk of vascular smooth muscle cell hyperproliferation, or preventing or reducing thickening of pulmonary artery walls using a compound as disclosed and described herein.

In some embodiments, methods are provided for the treatment of tumor angiogenesis, hemangiomas or tumor metastasis using a compound of having the structure of Formulae I, II or III.

In some embodiments, methods are provided for the treatment of breast cancer (including ductal carcinoma lobule carcinoma and breast epithelial cancer), ovarian cancer (including epithelial ovarian cancer), desmoid tumor, malignant peripheral nerve sheath cancer, acute leukemia, rhabdomyosarcoma. Ewing's sarcoma, extra-skeletal myxoid chondrosarcoma, or endometrial cancer using a compound of having the structure of Formulae I, II or III.

In some embodiments, methods are provided for the treatment of Wilms' tumor, esophageal adenocarcinoma, colon cancer, colorectal cancer, esophageal squamous cell carcinoma, lung adenocarcinoma, Epstein-Barr virus-negative gastric cancer, or pancreatic ductal adenocarcinoma using a compound of having the structure of Formulae I, II or III.

Some embodiments provide a method of evaluating the inhibition of EYA tyrosine phosphatase comprising contacting EYA tyrosine phosphatase with a compound of having the structure of Formulae I, II or III.

Some embodiments provide a method of evaluating a compound for preventing, reducing, or lowering the risk of vascular smooth muscle cell hyperproliferation, or preventing or reducing thickening of pulmonary artery walls, comprising contacting an EYA tyrosine phosphatase with a compound as disclosed and described herein and evaluating the results. Some embodiments provide a method of evaluating a compound for treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy, comprising contacting an EYA tyrosine phosphatase with a compound as disclosed and described herein and evaluating the results. Such embodiments include methods that comprise contacting an EYA tyrosine phosphatase with a compound having the structure of Formulae I, II, III or IV and evaluating the results.

Some embodiments provide a method of treating pulmonary arterial hypertension comprising contacting an EYA tyrosine phosphatase with a compound having the structure of Formulae I, II, III or IV and evaluating the results.

Some embodiments provide a method of evaluating a compound for inhibition of cell migration, comprising contacting an EYA tyrosine phosphatase with a compound having the structure of Formulae I, II or III and evaluating the results.

Some embodiments provide a method of evaluating a compound for inhibition of tumor angiogenesis, comprising contacting an EYA tyrosine phosphatase with a compound having the structure of Formulae I, II or III and evaluating the results.

Some embodiments provide a method of evaluating a compound for inhibition of tumor metastasis, comprising contacting an EYA tyrosine phosphatase with a compound having the structure of Formulae I, II or III and evaluating the results.

Some embodiments provide a method of evaluating a compound for inhibition of proliferative retinopathy, comprising contacting an EYA tyrosine phosphatase with a compound having the structure of Formulae I, II or III and evaluating the results.

Some embodiments provide a method of evaluating a compound for inhibition of retinopathy of prematurity, comprising contacting an EYA tyrosine phosphatase with a compound having the structure of Formulae I, II or III and evaluating the results.

Some embodiments provide a method of evaluating a compound for inhibition of diabetic retinopathy, comprising contacting an EYA tyrosine phosphatase with a compound having the structure of Formulae I, II or III and evaluating the results.

Some embodiments provide a method of evaluating a compound for inhibition of age related macular degeneration, comprising contacting an EYA tyrosine phosphatase with a compound having the structure of Formulae I, II or III and evaluating the results.

Some embodiments provide a method of evaluating a compound for inhibition of retinal vasculitis, comprising contacting an EYA tyrosine phosphatase with a compound having the structure of Formulae I, II or III and evaluating the results.

Some embodiments provide a method of evaluating a compound for inhibition of exudative vitreoretinopathy, comprising contacting an EYA tyrosine phosphatase with a compound having the structure of Formulae I, or II and evaluating the results.

Some embodiments provide a method of evaluating a compound for inhibition of hemangiomas comprising contacting an EYA tyrosine phosphatase with a compound having the structure of Formulae II or III and evaluating the results.

Some embodiments provide a method of evaluating a compound for inhibition of breast cancer (including ductal carcinoma lobule carcinoma and breast epithelial cancer) comprising contacting an EYA tyrosine phosphatase with a compound and evaluating the results.

Some embodiments provide a method of evaluating a compound for inhibition of ovarian cancer (including epithelial ovarian cancer) comprising contacting an EYA tyrosine phosphatase with a compound having the structure of Formulae I, II or III and evaluating the results.

Some embodiments provide a method of evaluating a compound for inhibition of desmoid tumor comprising contacting an EYA tyrosine phosphatase with a compound having the structure of Formulae I, II or III and evaluating the results.

Some embodiments provide a method of evaluating a compound for inhibition of malignant peripheral nerve sheath cancer comprising contacting an EYA tyrosine phosphatase with a compound having the structure of Formulae I, II or III and evaluating the results.

Some embodiments provide a method of evaluating a compound for inhibition of acute leukemia, rhabdomyosarcoma comprising contacting an EYA tyrosine phosphatase with a compound having the structure of Formulae I, II or III and evaluating the results.

Some embodiments provide a method of evaluating a compound for inhibition of Ewing's sarcoma comprising contacting an EYA tyrosine phosphatase with a compound having the structure of Formulae I, II or III and evaluating the results.

Some embodiments provide a method of evaluating a compound for inhibition of extra-skeletal myxoid chondrosarcoma comprising contacting an EYA tyrosine phosphatase with a compound having the structure of Formulae I, II or III and evaluating the results.

Some embodiments provide a method of evaluating a compound for inhibition of endometrial cancer comprising contacting an EYA tyrosine phosphatase with a compound having the structure of Formulae I, II or III and evaluating the results.

Some embodiments provide a method of evaluating a compound for inhibition of Wilms' tumor comprising contacting an EYA tyrosine phosphatase with a compound having the structure of Formulae I, II or III and evaluating the results.

Some embodiments provide a method of evaluating a compound for inhibition of esophageal adenocarcinoma comprising contacting an EYA tyrosine phosphatase with a compound having the structure of Formulae I, II or III and evaluating the results.

Some embodiments provide a method of evaluating a compound for inhibition of colon cancer comprising contacting an EYA tyrosine phosphatase with a compound having the structure of Formulae I, II or III and evaluating the results.

Some embodiments provide a method of evaluating a compound for inhibition of colorectal cancer comprising contacting an EYA tyrosine phosphatase with a compound having the structure of Formulae I, II or III and evaluating the results.

Some embodiments provide a method of evaluating a compound for inhibition of esophageal squamous cell carcinoma comprising contacting an EYA tyrosine phosphatase with a compound having the structure of Formulae I, II or III and evaluating the results.

Some embodiments provide a method of evaluating a compound for inhibition of lung adenocarcinoma comprising contacting an EYA tyrosine phosphatase with a compound having the structure of Formulae I, II or III and evaluating the results.

Some embodiments provide a method of evaluating a compound for inhibition of Epstein-Barr virus-negative gastric cancer comprising contacting an EYA tyrosine phosphatase with a compound having the structure of Formulae I, II or III and evaluating the results.

Some embodiments provide a method of evaluating a compound for inhibition of pancreatic ductal adenocarcinoma comprising contacting an EYA tyrosine phosphatase with a compound having the structure of Formulae I, II or III and evaluating the results.

In some embodiments, the results are evaluated by determining the level of inhibition of an EYA protein or truncated version thereof, relative to no inhibitor or relative to a control. In some embodiments, the results are evaluated by determining the level of inhibition of a full-length EYA protein or relative to a truncated EYA protein. In some embodiments, the results are evaluated by determining the level of reduction in pathological neovascularization relative to no inhibitor or relative to a control. In some embodiments, the results are evaluated by determining the level of reduction in angiogenesis relative to no inhibitor or relative to a control. In some embodiments, the results are evaluated by determining the level of reduction in metastasis to no inhibitor or relative to a control. In some embodiments, the results are evaluated by determining the level of reduction in tumor size relative to no inhibitor or relative to a control.

In certain aspects any of the embodiments providing methods said compound comprises a compound of any of the embodiments as disclosed and described herein.

In certain embodiments evaluating comprises evaluating the level of inhibition of full-length EYA tyrosine phosphatase in comparison with the level of inhibition of a truncated EYA tyrosine phosphatase. In certain embodiments, said truncated EYA tyrosine phosphatase comprises the catalytic domain of EYA tyrosine phosphatase. In certain embodiments, said truncated EYA tyrosine phosphatase comprises the catalytic domain (ED) of Eya3. In certain embodiments, said truncated EYA tyrosine phosphatase comprises the catalytic domain (ED) of Eya2.

In certain embodiments evaluating comprises evaluating the level of inhibition of EYA tyrosine phosphatase in comparison with the level of inhibition of a cysteine catalysis-based protein tyrosine phosphatase. In certain embodiments, said EYA tyrosine phosphatase comprises full-length EYA tyrosine phosphatase. In certain embodiments, said EYA tyrosine phosphatase comprises a truncated EYA tyrosine phosphatase which comprises the catalytic domain (ED) of EYA tyrosine phosphatase. In certain embodiments, said truncated EYA tyrosine phosphatase comprises the catalytic domain (ED) of Eya3. In certain embodiments, said truncated EYA tyrosine phosphatase comprises the catalytic domain (ED) of Eya2. In certain embodiments, said cysteine catalysis-based protein tyrosine phosphatase comprises PTP1B. In certain embodiments, said cysteine catalysis-based protein tyrosine phosphatase comprises FCP1. In certain embodiments, said cysteine catalysis-based protein tyrosine phosphatase comprises SCP. In certain embodiments, said cysteine catalysis-based protein tyrosine phosphatase comprises SH-PTP2. In certain embodiments, said cysteine catalysis-based protein tyrosine phosphatase comprises SH-PTP1.

### Definitions

Unless specific definitions are provided, the nomenclatures utilized in connection with, and the laboratory procedures and techniques of, analytical chemistry, synthetic organic chemistry, and medicinal and pharmaceutical chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, pharmaceutical preparation, formulation, and delivery, and treatment of patients.

As used herein, the following terms are defined with the following meanings, unless expressly stated otherwise.

The term “alkyl” refers to a branched or unbranched fully saturated acyclic aliphatic hydrocarbon group. An alkyl may be branched or straight chain. Alkyls may be substituted or unsubstituted. Alkyls include, but are not limited to, methyl, ethyl, propyl, isopropyl, butyl, isobutyl, tertiary butyl, pentyl, hexyl, and the like, each of which may be optionally substituted.

In certain embodiments, an alkyl comprises 1 to 20 carbon atoms (whenever it appears herein, a numerical range such as “1 to 20” refers to each integer in the given range, e.g., “1 to 20 carbon atoms” means that an alkyl group may comprise only 1 carbon atom, 2 carbon atoms, 3 carbon atoms, etc., up to and including 20 carbon atoms, although the term “alkyl” also includes instances where no numerical range of carbon atoms is designated). An alkyl may be designated as “C₁-C₆ alkyl” or similar designations. By way of example only, “C₁-C₄ alkyl” indicates an alkyl having one, two, three, or four carbon atoms. e.g., the alkyl is selected from methyl, ethyl, propyl, iso-propyl, butyl, iso-butyl, sec-butyl, and tert-butyl.

The term “alkenyl” used herein refers to a straight or branched chain aliphatic hydrocarbon of from two to twenty carbon atoms containing at least one carbon-carbon double bond including, but not limited to, 1-propenyl, 2-propenyl, 2-methyl-1-propenyl, 1-butenyl, 2-butenyl, and the like. In certain embodiments, an alkenyl comprises 2 to 20 carbon atoms (whenever it appears herein, a numerical range such as “2 to 20” refers to each integer in the given range; e.g., “2 to 20 carbon atoms” means that an alkenyl group may comprise only 2 carbon atoms, 3 carbon atoms, etc., up to and including 20 carbon atoms, although the term “alkenyl” also includes instances where no numerical range of carbon atoms is designated). An alkenyl may be designated as “C₂-C₆ alkenyl” or similar designations. By way of example only, “C₂-C₄ alkenyl” indicates an alkenyl having two, three, or four carbon atoms. e.g., the alkenyl is selected from ethenyl, propenyl, and butenyl.

The term “cycloalkyl” used herein refers to saturated aliphatic ring system having three to twenty carbon atoms. A cycloalkyl refers to monocyclic and polycyclic saturated aliphatic ring system including, but not limited to, cyclopropyl, cyclopentyl, cyclohexyl, cycloheptyl, bicyclo[4.4.0]decanyl, bicyclo[2.2.1]heptanyl, adamantyl, norbornyl, and the like. In certain embodiments, a cycloalkyl comprises 3 to 20 carbon atoms (whenever it appears herein, a numerical range such as “3 to 20” refers to each integer in the given range; e.g., “3 to 20 carbon atoms” means that a cycloalkyl group may comprise only 3 carbon atoms, etc., up to and including 20 carbon atoms, although the term “cycloalkyl” also includes instances where no numerical range of carbon atoms is designated). A cycloalkyl may be designated as “C₃-C₇ cycloalkyl” or similar designations. By way of example only. “C₃-C₆ cycloalkyl” indicates a cycloalkyl having two, three, four, five or six carbon atoms, e.g., the cycloalkyl is selected from cyclopropyl, cyclobutyl, cyclopentyl, and cyclohexyl.

The term “cycloalkenyl” used herein refers to aliphatic ring system having three to twenty carbon atoms having at least one carbon-carbon double bond in the ring. A cycloalkenyl refers to monocyclic and polycyclic unsaturated aliphatic ring system including, but are not limited to, cyclopropenyl, cyclopentenyl, cyclohexenyl, cycloheptenyl, bicyclo[3.1.0]hexyl, norbornylenyl, 1,1′-bicyclopentenyl, and the like. In certain embodiments, a cycloalkenyl comprises 3 to 20 carbon atoms (whenever it appears herein, a numerical range such as “3 to 20” refers to each integer in the given range; e.g., “3 to 20 carbon atoms” means that a cycloalkenyl group may comprise only 3 carbon atoms, etc., up to and including 20 carbon atoms, although the term “cycloalkenyl” also includes instances where no numerical range of carbon atoms is designated). A cycloalkenyl may be designated as “C₃-C₇ cycloalkenyl” or similar designations. By way of example only, “C₃-C₆ cycloalkenyl” indicates an alkenyl having two, three, four, five or six carbon atoms, e.g., the cycloalkyl is selected from cyclopropenyl, cyclobutenyl, cyclopentenyl, and cyclohexenyl.

The term “alkoxy” used herein refers to straight or branched chain alkyl covalently bonded to oxygen where the “alkoxy” is attached to the parent molecule through at least an oxygen linkage. Where an “alkoxy” substituent requires two points of attachment to the rest of the molecule the “alkoxy” is attached to the parent molecule through an oxygen linkage and a carbon linkage. Examples of alkoxy groups include, but are not limited to, methoxy, ethoxy, propoxy, isopropoxy, butoxy, n-butoxy, sec-butoxy, t-butoxy and the like. An alkoxy may be designated as “C₁-C₆ alkoxy” or similar designations. By way of example only. “C₁-C₄ alkoxy” indicates an alkyl having one, two, three, or four carbon atoms, e.g., the alkoxy is selected from methoxy, ethoxy, propoxy, iso-propoxy, butoxy, iso-butoxy, sec-butoxy, and tert-butoxy.

The term “heteroalkyl” refers to a group comprising at least one alkyl or alkenyl, and one or two heteroatoms. Where a “heteroalkyl” substituent requires two points of attachment to the rest of the molecule the “heteroalkyl” is attached to the parent molecule through a heteroatom linkage and a carbon linkage, a first carbon linkage and a second carbon linkage, or a first heteroatom linkage and a second heteroatom linkage. Examples of heteroalkyls include, but are not limited to, —CH₂OCH₂CH₂—, —CH₂OCH₂—, —CH₂CH₂OCH₂CH₂—, —CH₂OCH═CH—, —CH═CHOCH═CH—, —OCH₂O—, —CH₂NHCH₂CH₂—, —CH₂NHCH₂—, —CH₂CH₂NHCH₂CH₂—, —NHCH═CH—, —NHCH₂CH₂—, —N═CHCH₂—, —CH₂NHCH═CH—, —CH═CHNHCH═CH—, —NHCH₂NH—, and the like.

The term “heterocyclic” or “heterocyclyl” used herein refers to a cyclic ring system radical having at least one non-aromatic ring in which one or more ring atoms are not carbon, namely heteroatom. Monocyclic “heterocyclic” or “heterocyclyl” moieties are non-aromatic. Bicyclic “heterocyclic” or “heterocyclyl” moieties include one non-aromatic ring wherein at least one heteroatom is present in a ring. Tricyclic “heterocyclic” or “heterocyclyl” moieties include at least one non-aromatic ring wherein at least one heteroatom is present in a ring. Examples of heterocyclic groups include, but are not limited to, piperidinyl, piperazinyl, morpholinyl, tetrahydrofuranyl, dioxolanyl, tetrahydropyranyl, pyrrolidinyl, and the like.

The term “heteroatom” refers to an atom other than carbon or hydrogen. Heteroatoms are typically independently selected from oxygen, sulfur, nitrogen, and phosphorus, but are not limited to those atoms. In embodiments in which two or more heteroatoms are present, the two or more heteroatoms may all be the same as one another, or some or all of the two or more heteroatoms may each be different from the others.

The term “aryl” refers to an aromatic group wherein each of the atoms forming the ring is a carbon atom. Examples of aryl groups include, but are not limited to phenyl, and naphthalenyl. In certain embodiments, a phenyl group is substituted at one or more positions. Examples of aryl groups comprising substitutions include, but are not limited to, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl, 3-cyanophenyl, 4-cyanophenyl, dimethylphenyl, hydroxynaphthyl, hydroxymethylphenyl. (trifluoromethyl)phenyl, and 4-morpholin-4-ylphenyl.

The term “heteroaryl” refers to an aromatic mono-, bi- or tricyclic ring system wherein at least one atom forming the aromatic ring system is a heteroatom. Heteroaryl rings may be formed by three, four, five, six, seven, eight, nine, or more than nine atoms. Heteroaryl groups may be optionally substituted. Examples of heteroaryl groups include, but are not limited to, aromatic C s heterocyclic groups comprising one oxygen or sulfur atom or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl groups are optionally substituted with one or more substituents, independently selected from halo, hydroxy, amino, cyano, nitro, alkylamido, acyl, C₁₋₆-alkoxy. C₁₋₆-alkyl, C₁₋₆-hydroxyalkyl, C₁₋₆-aminoalkyl, C₁₋₆-alkylamino, alkylsulfenyl, alkylsulfonyl, alkylsulfonyl, sulfamoyl, or trifluoromethyl. In some embodiments, the substituents are halo, hydroxy, cyano, O—C₁₋₆-alkyl, C₁₋₆-alkyl, hydroxy-C₁₋₆-alkyl, and amino-C₁₋₆-alkyl. Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline.

The term “arylalkyl” refers to a group comprising an aryl group bound to an alkyl group. Examples of arylalkyl groups include, but are not limited to, benzyl phenethyl, phenpropyl, phenbutyl, and the like. In some embodiments, arylalkyls may be substituted or unsubstituted, and can be substituted on either the aryl or alkyl portion or on both. Where an “arylalkyl” substituent requires two points of attachment to the rest of the molecule the “arylalkyl” can be attached to the parent molecule through a carbon linkage in the aryl group and a carbon linkage in the alkyl group.

The term “heteroarylalkyl” used herein refers to one or more heteroaryl groups appended to an alkyl radical. Examples of heteroarylalkyl include, but are not limited to, pyridylmethyl, furanylmethyl, thiopheneylethyl, and the like. In some embodiments, heteroarylalkyls may be substituted or unsubstituted, and can be substituted on either the heteroaryl or alkyl portion or on both. Where an “heteroarylalkyl” substituent requires two points of attachment to the rest of the molecule the “heteroarylalkyl” can be attached to the parent molecule through a carbon linkage in the heteroaryl group and a carbon linkage in the alkyl group.

The term “heterocyclylalkyl” used herein refers to one or more heterocyclyl groups appended to an alkyl radical. Examples of heterocyclylalkyl include, but are not limited to, piperidinylmethyl, piperidinylethyl, morpholinylmethyl, morpholinylethyl, and the like.

The term “(cycloalkyl)alkyl” used herein refers to one or more cycloalkyl groups appended to an alkyl radical. Examples of (cycloalkyl)alkyl include, but are not limited to, cyclohexylmethyl, cyclohexylethyl, cyclopentylmethyl, cyclopentylethyl, and the like. In some embodiments, (cycloalkyl)alkyl may be substituted or unsubstituted.

Unless otherwise indicated, the term “optionally substituted,” refers to a group in which none, one, or more than one of the hydrogen atoms has been replaced with one or more group(s) individually and independently selected from: alkyl, alkenyl, cycloalkenyl, cycloalkyl, aryl, arylalkyl, heteroaryl, heterocyclyl, hydroxy, alkoxy, cyano, halo, oxo, thiocarbonyl, ester, O-carbamyl, N-carbamyl, O-thiocarbamyl, N-thiocarbamyl, C-amido, N-amido, S-sulfonamido. N-sulfonamido, C-carboxy, O-carboxy, isocyanato, thiocyanato, isothiocyanato, nitro, and amino, including mono- and di-substituted amino groups, and the protected derivatives of amino groups. Such protective derivatives (and protecting groups that may form such protective derivatives) are known to those of skill in the art and may be found in references such as Greene and Wuts, above. When the group contains a nitrogen, or a sulfur, an oxo as a substituent also includes oxides, for example pyridine-N-oxide, thiopyran sulfoxide and thiopyran-S,S-dioxide. In embodiments in which two or more hydrogen atoms have been substituted, the substituent groups may together form a ring.

The substituent “R” appearing by itself and without a number designation refers to a substituent selected from H (hydrogen), alkyl, cycloalkyl, aryl, heteroaryl (bonded through a ring carbon) and heterocyclyl (bonded through a ring carbon).

The term “O-carboxy” refers to the group consisting of formula RC(═O)O—.

The term “C-carboxy” refers to the group consisting of formula —C(═O)OR.

The term “cyano” refers to the group consisting of formula —CN.

The term “isocyanato” refers to the group consisting of formula —N═C═O.

The term “thiocyanato” refers to the group consisting of formula —CNS.

The term “isothiocyanato” refers to the group consisting of formula —N═C═S.

The term “sulfonyl” refers to the group consisting of formula —S(═O)—R.

The term “S-sulfonamido” refers to the group consisting of formula —S(═O)₂NR.

The term “N-sulfonamido” refers to the group consisting of formula RS(═O)₂NH—.

The term “O-carbamyl” refers to the group consisting of formula —OC(═O)—NR.

The term “N-carbamyl” refers to the group consisting of formula ROC(═O)NH—.

The term “O-thiocarbamyl” refers to the group consisting of formula —OC(═S)—NR.

The term “N-thiocarbamyl” refers to the group consisting of formula ROC(═S)NH—.

The term “C-amido” refers to the group consisting of formula —C(═O)—NR₂.

The term “N-amido” refers to the group consisting of formula RC(═O)NH—.

The term “oxo” refers to the group consisting of formula ═O.

The term “ester” refers to a chemical moiety with formula —(R)ₙ—C(═O)OR′, where R and R′ are independently selected from alkyl, cycloalkyl, aryl, heteroaryl (bonded through a ring carbon) and non-aromatic heterocycle (bonded through a ring carbon), where n is 0 or 1.

The term “amino” refers to a chemical moiety with formula —NHR′R″, where R′ and R″ are each independently selected from hydrogen, alkyl, cycloalkyl, aryl, heteroaryl (bonded through a ring carbon) and heteroalicyclic (bonded through a ring carbon).

The term “stereoisomers” as used herein means isomers that possess identical constitution, but which differ in the arrangement of their atoms in space. Including, for example, all enantiomers, diastereomers, geometric isomers, and atropisomers.

Wherever a substituent as depicted as a di-radical (i.e., has two points of attachment to the rest of the molecule), it is to be understood that the substituent can be attached in any directional configuration unless otherwise indicated. Thus, for example, a substituent depicted as -AE- or

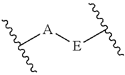

includes the substituent being oriented such that the A is attached at the leftmost attachment point of the molecule as well as attached at the rightmost attachment point of the molecule.

It is to be understood that certain radical naming conventions can include either a mono-radical or a di-radical, depending on the context. For example, where a substituent requires two points of attachment to the rest of the molecule, it is understood that the substituent is a di-radical. A substituent identified as alkyl, that requires two points of attachment, includes di-radicals such as —CH₂—, —CH₂CH₂—, —CH₂CH(CH₃)CH₂—, and the like; a substituent depicted as alkoxy that requires two points of attachment, includes di-radicals such as —OCH₂—, —OCH₂CH₂—, —OCH₂CH(CH₃)CH₂—, and the like; and a substituent identified as arylalkyl that requires two points of attachment, includes di-radicals such as

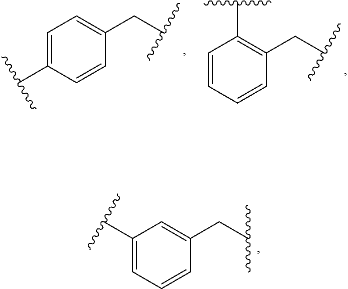 [SMILES: *Cc1ccc(*)cc1 ; *Cc1ccccc1* ; *Cc1cccc(*)c1]

and the like.

Throughout the specification, groups and substituents thereof can be chosen by one skilled in the field to provide stable moieties and compounds.

The term “pharmaceutical agent” refers to a chemical compound or composition capable of inducing a desired therapeutic effect in a patient. In certain embodiments, a pharmaceutical agent comprises an active agent, which is the agent that induces the desired therapeutic effect. In certain embodiments, a pharmaceutical agent comprises a prodrug. In certain embodiments, a pharmaceutical agent comprises inactive ingredients such as carriers, excipients, and the like.

The term “therapeutically effective amount” refers to an amount of a pharmaceutical agent sufficient to achieve a desired therapeutic effect.

The term “pharmaceutically acceptable” refers to a formulation of a compound that does not significantly abrogate the biological activity, a pharmacological activity and/or other properties of the compound when the formulated compound is administered to a patient. In certain embodiments, a pharmaceutically acceptable formulation does not cause significant irritation to a patient.

The term “co-administer” refers to administering more than one pharmaceutical agent to a patient. In certain embodiments, co-administered pharmaceutical agents are administered together in a single dosage unit. In certain embodiments, co-administered pharmaceutical agents are administered separately. In certain embodiments, co-administered pharmaceutical agents are administered at the same time. In certain embodiments, co-administered pharmaceutical agents are administered at different times.

The term “patient” includes human and animal subjects.

The term “substantially pure” means an object species (e.g., compound) is the predominant species present (i.e., on a molar basis it is more abundant than any other individual species in the composition). In certain embodiments, a substantially purified fraction is a composition wherein the object species comprises at least about 50 percent (on a molar basis) of all species present. In certain embodiments, a substantially pure composition will comprise more than about 80%, 85%, 90%, 95%, or 99% of all species present in the composition. In certain embodiments, the object species is purified to essential homogeneity (contaminant species cannot be detected in the composition by conventional detection methods) wherein the composition consists essentially of a single species.

### Certain Compounds

Certain compounds that modulate EYA tyrosine phosphatase and/or bind to EYA tyrosine phosphatase play a role in health. In certain embodiments, compounds are useful for treating diseases or conditions as provided elsewhere herein.

One of skill in the art will recognize that analogous synthesis schemes may be used to synthesize similar compounds. One of skill will recognize that compounds of the present embodiments may be synthesized using other synthesis schemes. In certain embodiments, a salt corresponding to any of the compounds provided herein is provided.

In certain embodiments, a salt corresponding to a compound as disclosed and described herein is provided. In certain embodiments, a salt is obtained by reacting a compound with an acid, such as hydrochloric acid, hydrobromic acid, sulfuric acid, nitric acid, phosphoric acid, methanesulfonic acid, ethanesulfonic acid, p-toluenesulfonic acid, salicylic acid, and the like. In certain embodiments, a salt is obtained by reacting a compound with a base to form a salt such as an ammonium salt, an alkali metal salt, such as a sodium or a potassium salt, an alkaline earth metal salt, such as a calcium or a magnesium salt, a salt of organic bases such as choline, dicyclohexylamine, N-methyl-D-glucamine, tris(hydroxymethyl)methylamine, 4-(2-hydroxyethyl)-morpholine, 1-(2-hydroxyethyl)-pyrrolidine, ethanolamine and salts with amino acids such as arginine, lysine, and the like. In certain embodiments, a salt is obtained by reacting a free acid form of a compound as disclosed and described herein with multiple molar equivalents of a base, such as bis-sodium, bis-ethanolamine, and the like.

In certain embodiments, a salt corresponding to a compound of the present embodiments is selected from acetate, ammonium, benzenesulfonate, benzoate, bicarbonate, bisulfate, bitartrate, borate, bromide, calcium edetate, camsylate, carbonate, chloride, cholinate, clavulanate, citrate, dihydrochloride, diphosphate, edetate, edisylate, estolate, esylate, fumarate, gluceptate, gluconate, glutamate, glycollylarsanilate, hexylresorcinate, hydrabanine, hydrobromide, hydrochloride, hydroxynaphthoate, iodide, isethionate, lactate, lactobionate, laurate, magnesium, malate, maleate, mandelate, mucate, napsylate, nitrate, N-methylglucamine, oxalate, pamoate (embonate), palmitate, pantothenate, phosphate, polygalacturonate, potassium, salicylate, sodium, stearate, subaceatate, succinate, sulfate, tannate, tartrate, teoclate, tosylate, triethiodide, tromethamine, trimethylammonium, and valerate salts.

### Methods of Screening for EYA Tyrosine Phosphatase Inhibition

Also provided herein are methods of screening compounds for EYA tyrosine phosphatase inhibition. These methods can include methods of evaluating inhibitory properties of a compound, such as, but not limited to, a member of the set of compounds provided herein. In certain embodiments, the methods can comprise contacting EYA tyrosine phosphatase with a compound, and evaluating the level of EYA tyrosine phosphatase inhibition. In certain embodiments, the compound is a compound as disclosed herein.

The EYA tyrosine phosphatase can be from any organism that expresses EYA tyrosine phosphatases, such as those that are known in the art. In some embodiments, the EYA tyrosine phosphatase is from a mammalian organism, such as human, primate, bovine, equine, porcine, ovine, murine, canine or feline EYA tyrosine phosphatase. In some embodiments the EYA tyrosine phosphatase is from a non-mammalian organism, such as avian or zebrafish EYA tyrosine phosphatase. In typical embodiments, the EYA tyrosine phosphatase is from human or primate EYA tyrosine phosphatase. In some embodiments, the EYA tyrosine phosphatase is from a non-mammalian organism, such as zebrafish and the like. Cloning and expression of EYA tyrosine phosphatases from various organisms can be performed as described herein or as otherwise known in the art.

In some embodiments, the EYA tyrosine phosphatase is a full-length EYA tyrosine phosphatase. Thus, in some embodiments, full-length EYA tyrosine phosphatase can comprise a full-length isoform of EYA tyrosine phosphatase. The full-length EYA tyrosine phosphatase can be full-length Eya1, Eya2, Eya3, and Eya4, or an isoform thereof.

In some embodiments, the EYA tyrosine phosphatase is a truncated EYA tyrosine phosphatase. In some embodiments, the truncated EYA tyrosine phosphatase comprises one or more of N-terminal, C-terminal, or internal deletions from a full-length isoform of EYA tyrosine phosphatase. In some embodiments, the truncated EYA tyrosine phosphatase comprises a truncated Eya1, Eya2. Eya3, and Eya4, or an isoform thereof. For example, in certain embodiments, the EYA tyrosine phosphatase comprises the catalytic domain (ED) of EYA tyrosine phosphatase. As will be understood by those of skill in the art, the catalytic domain (ED) of EYA tyrosine phosphatase can be at least 80%, 85%, 90%, 91%, 92%, 93%, 94%, 95%, 96%, 97%, 98%, 99%, or 100% identical to the ED of Eya3, as can be identified by those of skill in the art. For example, the ED of Eya3 can be least 80%, 85%, 90%, 91%, 92%, 93%, 94%, 95%, 96%, 97%, 98%, 99%, or 100% identical to residues 223-510 of mouse Eya3, or to an art-understood aligned corresponding ED for Eya1. Eya2, Eya3 or Eya4. It will be appreciated by those of skill in the art that corresponding ED sequences can be found using software known in the art, for example. ClustalW.

In certain embodiments, the methods can comprise evaluating the level of EYA tyrosine phosphatase inhibition using a cell-free assay as described herein or otherwise known in the art. For example, in certain embodiments, the method comprises measuring inhibition of phosphatase activity using a p-nitrophenylphosphate (pNPP) assay as described herein or otherwise known in the art. In certain embodiments, the method comprises a peptide-based phosphatase assay as described herein or otherwise known in the art. Inhibition can be determined by whether tyrosine phosphatase activity is reduced according to a user-selected level, as described herein or otherwise known in the art. Thus, in some embodiment, a user-selected level of inhibition can be, for example, at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% inhibition of tyrosine phosphatase activity. In some embodiments, a user-selected level of inhibition can be an IC₅₀ value that is, for example, less than 10 mM, 1 mM, 100 μM, 90 μM, 80 μM, 70 μM, 60 μM, 50 μM, 40 μM, 30 μM, 20 μM, 10 μM, 1 μM, 0.1 μM, 0.01 μM, or less than 0.001 μM, as described herein or otherwise known in the art.

The results of the methods of evaluating the inhibitory properties of the compounds provided herein can be reported in terms understood in the art including, for example, IC₅₀, EC₅₀, Kᵢ, or other standard terms known in the art. Thus, the evaluation methods provided herein can include evaluating the results where evaluating the results includes determining the inhibitory properties of the compound(s) being tested. In some embodiments evaluating the results also includes comparing the inhibitory properties of a compound being tested to the inhibitory properties of one or more reference compounds.

In certain embodiments, the methods can comprise an in vitro assay on whole cells as described herein or otherwise known in the art. For example, in certain embodiments, the method comprises measuring inhibition of cell migration using a cell migration assay as described herein or otherwise known in the art. In some embodiments, the method comprises measuring inhibition of tubulogenesis as described herein or otherwise known in the art. In some embodiments, the method comprises measuring inhibition of angiogenesis as described herein or otherwise known in the art.

In certain embodiments, the methods can comprise an in vivo assay as described herein or otherwise known in the art. For example, in certain embodiments, the method comprises treating an animal with a compound provided herein, and evaluating the effects of treating the animal with the compound. In certain embodiments, the method comprises using an animal model for preventing, reducing, lowering the risk of, or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy. In certain embodiments, the method comprises using an animal model for preventing, reducing, or lowering the risk of vascular smooth muscle cell hyperproliferation, or preventing or reducing thickening of pulmonary artery walls. In certain embodiments, the method comprises using an animal model for proliferative retinopathy, retinopathy of prematurity, diabetic retinopathy, age related macular degeneration, retinal vasculitis, exudative vitreoretinopathy, tumor angiogenesis, hemangiomas or tumor metastasis. For example, in some embodiments, the method comprises measuring inhibition of vasculature formation in vivo as described herein or otherwise known in the art. For example, the method can comprise measurement of angiogenesis in zebrafish embryos as described herein or otherwise known in the art. In some embodiments, the method can comprise measurement of angiogenesis in a retinal angiogenesis model in postnatal mice as described herein or otherwise known in the art. For example, the method can comprise measurement of angiogenesis in a mouse model of oxygen-induced retinopathy as described herein or otherwise known in the art. In some embodiments, the method can comprise measurement of tumor growth. For example, the method can comprise measurement of tumor growth in a xenograft mouse model as described herein or otherwise known in the art.

### Methods of Comparing Inhibition of Full-Length EYA Inhibition to ED Inhibition

Also presented herein is a method for evaluating the inhibition of EYA tyrosine phosphatase comprising contacting a full-length EYA tyrosine phosphatase with a compound from a library of compounds and evaluating the results; wherein the compound has a user selected relative level of inhibitory activity compared to the inhibitory activity of the same compound when it contacts the catalytic domain (ED) of EYA tyrosine phosphatase. In some embodiments, the user-selected relative level of inhibition of full-length EYA tyrosine phosphatase at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 115%, 120%, 125%, 130%, 135%, 140%, 145%, 150%, 155%, 160%, 165%, 170%, 175%, 180%, 185%, 190%, 200%, 300%, 400% or at least 500% as much inhibitory activity as the same compound when it contacts the catalytic domain (ED) of EYA tyrosine phosphatase.

The full-length or ED form of EYA tyrosine phosphatase can be from any organism that expresses EYA tyrosine phosphatases, such as those that are known in the art. In some embodiments, the EYA tyrosine phosphatase is from a mammalian organism, such as human, primate, bovine, equine, porcine, ovine, murine, canine or feline EYA tyrosine phosphatase. In some embodiments the EYA tyrosine phosphatase is from a non-mammalian organism, such as avian or zebrafish EYA tyrosine phosphatase. In typical embodiments, the EYA tyrosine phosphatase is from human or primate EYA tyrosine phosphatase. In some embodiments, the EYA tyrosine phosphatase is from a non-mammalian organism, such as zebrafish and the like. Cloning and expression of EYA tyrosine phosphatases from various organisms can be performed as described herein or as otherwise known in the art.

The full-length EYA tyrosine phosphatase can comprise a full-length isoform of EYA tyrosine phosphatase. The full-length EYA tyrosine phosphatase can be full-length Eya1, Eya2, Eya3, and Eya4, or an isoform thereof.

As will be understood by those of skill in the art, the catalytic domain (ED) of EYA tyrosine phosphatase can be at least 80%, 85%, 90%, 91%, 92%, 93% 94%, 95% 96% 97%, 98%, 99%, or 100% identical to the ED of Eya3, as can be identified by those of skill in the art. For example, the ED of Eya3 can be least 80%, 85%, 90%, 91%, 92%, 93%, 94%, 95%, 96%, 97%, 98%, 99%, or 100% identical to residues 223-510 of mouse Eya3, or to an art-understood aligned corresponding ED for Eya1, Eya2, Eya3 or Eya4. It will be appreciated by those of skill in the art that corresponding ED sequences can be found using software known in the art, for example, ClustalW.

In certain embodiments, the methods can comprise evaluating the level of EYA tyrosine phosphatase inhibition using a cell-free assay as described herein or otherwise known in the art. For example, in certain embodiments, the method comprises measuring inhibition of phosphatase activity using a p-nitrophenylphosphate (pNPP) assay as described herein or otherwise known in the art. In certain embodiments, the method comprises a peptide-based phosphatase assay as described herein or otherwise known in the art. Inhibition can be determined by whether tyrosine phosphatase activity is reduced according to a user-selected level, as described herein or otherwise known in the art. Thus, in some embodiment, a user-selected level of inhibition can be, for example, at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% inhibition of tyrosine phosphatase activity. In some embodiments, a user-selected level of inhibition can be an IC₅₀ value that is, for example, less than 10 mM, 1 mM, 100 μM, 90 μM, 80 μM, 70 μM, 60 μM, 50 μM, 40 μM, 30 μM, 20 μM, 10 μM, 1 μM, 0.1 μM, 0.01 μM, or less than 0.001 μM, as described herein or otherwise known in the art.

The results of the methods of evaluating the inhibitory properties of the compounds provided herein can be reported in terms understood in the art including, for example, IC₅₀, EC₅₀, Kᵢ, or other standard terms known in the art. Thus, the evaluation methods provided herein can include evaluating the results where evaluating the results includes determining the inhibitory properties of the compound(s) being tested. In some embodiments evaluating the results also includes comparing the inhibitory properties of a compound being tested to the inhibitory properties of one or more reference compounds.

Also presented herein is a method for evaluating the inhibition of EYA tyrosine phosphatase comprising: a) contacting the catalytic domain (ED) of EYA tyrosine phosphatase with a compound from a library of compounds and evaluating the results; and b) contacting a full-length EYA tyrosine phosphatase with the compound and evaluating the results. The method can further comprise: c) performing a) for each compound in the library of compounds; d) selecting one or more compounds from c) that inhibit the catalytic domain (ED) of EYA tyrosine phosphatase according to a user-selected level; e) performing b) for each compound selected in d); and f) selecting one or more compounds from e) that inhibit full-length EYA tyrosine phosphatase according to a user-selected level.

The full-length or ED form of EYA tyrosine phosphatase can be from any organism that expresses EYA tyrosine phosphatases, such as those that are known in the art. In some embodiments, the EYA tyrosine phosphatase is from a mammalian organism, such as human, primate, bovine, equine, porcine, ovine, murine, canine or feline EYA tyrosine phosphatase. In some embodiments the EYA tyrosine phosphatase is from a non-mammalian organism, such as avian or zebrafish EYA tyrosine phosphatase. In typical embodiments, the EYA tyrosine phosphatase is from human or primate EYA tyrosine phosphatase. In some embodiments, the EYA tyrosine phosphatase is from a non-mammalian organism, such as zebrafish and the like. Cloning and expression of EYA tyrosine phosphatases from various organisms can be performed as described herein or as otherwise known in the art.

The full-length EYA tyrosine phosphatase can comprise a full-length isoform of EYA tyrosine phosphatase. The full-length EYA tyrosine phosphatase can be full-length Eya1, Eya2, Eya3, and Eya4, or an isoform thereof.

As will be understood by those of skill in the art, the catalytic domain (ED) of EYA tyrosine phosphatase can be at least 80%, 85%, 90%, 91%, 92%, 93%, 94%, 95%, 96%, 97%, 98%, 99%, or 100% identical to the ED of Eya3, as can be identified by those of skill in the art. For example, the ED of Eya3 can be least 80%, 85%, 90%, 91%, 92%, 93%, 94%, 95%, 96%, 97%, 98%, 99%, or 100% identical to residues 223-510 of mouse Eya3, or to an art-understood aligned corresponding ED for Eya1, Eya2, Eya3 or Eya4. It will be appreciated by those of skill in the art that corresponding ED sequences can be found using software known in the art, for example, ClustalW.

In certain embodiments, the methods can comprise evaluating the level of EYA tyrosine phosphatase inhibition using a cell-free assay as described herein or otherwise known in the art. For example, in certain embodiments, the method comprises measuring inhibition of phosphatase activity using a p-nitrophenylphosphate (pNPP) assay as described herein or otherwise known in the art. In certain embodiments, the method comprises a peptide-based phosphatase assay as described herein or otherwise known in the art. Inhibition can be determined by whether tyrosine phosphatase activity is reduced according to a user-selected level, as described herein or otherwise known in the art. Thus, in some embodiment, a user-selected level of inhibition can be, for example, at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% inhibition of tyrosine phosphatase activity. In some embodiments, a user-selected level of inhibition can be an IC₅₀ value that is, for example, less than 10 mM, 1 mM, 100 μM, 90 μM, 80 μM, 70 μM, 60 μM, 50 μM, 40 μM, 30 μM, 20 μM, 10 μM, 1 μM, 0.1 μM, 0.01 μM, or less than 0.001 μM, as described herein or otherwise known in the art.

The results of the methods of evaluating the inhibitory properties of the compounds provided herein can be reported in terms understood in the art including, for example IC₅₀, EC₅₀, Kᵢ, or other standard terms known in the art. Thus, the evaluation methods provided herein can include evaluating the results where evaluating the results includes determining the inhibitory properties of the compound(s) being tested. In some embodiments evaluating the results also includes comparing the inhibitory properties of a compound being tested to the inhibitory properties of one or more reference compounds.

In any of the above methods, the inhibitory activity of a compound towards full-length EYA tyrosine phosphatase can be compared to the inhibitory activity of a compound towards the catalytic domain (ED) of EYA tyrosine phosphatase. The comparison can be based on any measure of inhibition as described herein or as otherwise known in the art. For example, in some embodiments, a comparison is made based on the inhibition of full-length EYA tyrosine phosphatase versus inhibition of a the catalytic domain (ED) of EYA tyrosine phosphatase at a given concentration of a compound. The comparison can be expressed in terms as described herein or otherwise known in the art, such as percent difference or fold difference. For example, a compound at a given concentration may inhibit an full-length EYA tyrosine phosphatase with 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 115%, 120%, 125%, 130%, 135%, 140%, 145%, 150%, 155%, 160%, 165%, 170%, 175%, 180%, 185%, 190%, 200%, 300%, 400% or greater than 500% as much inhibitory activity as compared to its inhibition of catalytic domain (ED) of EYA tyrosine phosphatase at the same concentration of the compound. Likewise, a compound at a given concentration may inhibit full-length EYA tyrosine phosphatase with 0.01 fold, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or greater than 1000 fold as much inhibitory activity as compared to its inhibition of catalytic domain (ED) of EYA tyrosine phosphatase at the same concentration of the compound. Similarly, the comparison may be made by comparing the IC₅₀ of a compound towards full-length EYA tyrosine phosphatase with the IC₅₀ of the same compound towards the catalytic domain (ED) of EYA tyrosine phosphatase. For example, a compound may inhibit full-length EYA tyrosine phosphatase with the IC₅₀ that is 2 fold, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or greater than 100 fold of the IC₅₀ of the compound towards the catalytic domain (ED) of EYA tyrosine phosphatase.

In some embodiments, the method further comprises selecting a compound based on a comparison of on the inhibition of full-length EYA tyrosine phosphatase versus inhibition of the catalytic domain (ED) of EYA tyrosine phosphatase. Typically, the compound will be selected as a specific inhibitor of an EYA tyrosine phosphatase when it exhibits inhibition of full-length EYA tyrosine phosphatase that shows greater selectivity compared to the catalytic domain (ED) of EYA tyrosine phosphatase. Thus, for example, in some embodiments, a compound may be selected as an EYA tyrosine phosphatase inhibitor if the IC₅₀ towards full-length EYA tyrosine phosphatase that is 2 fold, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 fold lower than the IC₅₀ of that compound towards the catalytic domain (ED) of EYA tyrosine phosphatase.

Methods of Comparing EYA Tyrosine Phosphatase Inhibition with Inhibition of Other Classes of Tyrosine Phosphatases.

Also presented herein is a method for identifying a compound that specifically inhibits EYA tyrosine phosphatase comprising: a) contacting EYA tyrosine phosphatase with a compound and evaluating the results; and b) contacting a cysteine catalysis-based protein tyrosine phosphatase or an FCP/SCP family protein tyrosine phosphatase with the compound and evaluating the results.

In some embodiments, the results of contacting EYA tyrosine phosphatase are compared to the results of contacting a cysteine catalysis-based protein tyrosine phosphatase with a compound. Cysteine catalysis-based protein tyrosine phosphatases are a class of protein tyrosine phosphatases as described by Alonso et al. ((2004) Cell. 117:699-711, hereby incorporated by reference in its entirety) or otherwise known in the art. While not intending to be limited to the following, it is postulated that that the EYA tyrosine phosphatase domain differs mechanistically from other protein tyrosine phosphatases such as cysteine catalysis-based protein tyrosine phosphatases, which utilize a cysteine residue in catalysis. Instead, it is postulated that the EYAs employ an aspartate as a nucleophile and another conserved aspartate two residues downstream as an acid catalyst. Thus, a comparison of EYA tyrosine phosphatase inhibition with the inhibition of a protein tyrosine phosphatase from another class can define specificity for the EYA tyrosine phosphatase active site, for example. In some embodiments, the cysteine catalysis-based protein tyrosine phosphatase is PTP1B. In some embodiments, the cysteine catalysis-based protein tyrosine phosphatase is SH-PTP1. In some embodiments, the cysteine catalysis-based protein tyrosine phosphatase is SH-PTP2. In some embodiments, the cysteine catalysis-based protein tyrosine phosphatase is another cysteine catalysis-based protein tyrosine phosphatase, as are known in the art.

In some embodiments, the results of contacting EYA tyrosine phosphatase with a compound are compared to the results of contacting a FCP/SCP family protein tyrosine phosphatase with a compound. While not intending to be limited to the following, it is postulated that that FCP/SCP family protein tyrosine phosphatases are a family of aspartate-based protein tyrosine phophatases. Thus, a comparison of EYA tyrosine phosphatase inhibition with the inhibition of a protein tyrosine phosphatase from another protein tyrosine phosphatase family can define specificity for the EYA tyrosine phosphatase active site, for example. In some embodiments, the FCP/SCP family protein tyrosine phosphatase is FCP1. In some embodiments, the FCP/SCP family protein tyrosine phosphatase is SCP.

In some embodiments, the results of contacting EYA tyrosine phosphatase are compared to the results of contacting a cysteine catalysis-based protein tyrosine phosphatase or a FCP/SCP family protein tyrosine phosphatase with a compound. The comparison can be based on any measure of inhibition as described herein or as otherwise known in the art. For example, in some embodiments, a comparison is made based on the inhibition of EYA tyrosine phosphatase versus inhibition of a cysteine catalysis-based protein tyrosine phosphatase or a FCP/SCP family protein tyrosine phosphatase at a given concentration of a compound. The comparison can be expressed in terms as described herein or otherwise known in the art, such as percent difference or fold difference. For example, a compound at a given concentration may inhibit an EYA tyrosine phosphatase with 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 115%, 120%, 125%, 130%, 135%, 140%, 145%, 150%, 155%, 160%, 165%, 170%, 175%, 180%, 185%, 190%, 200%, 300%, 400% or greater than 500% as much inhibitory activity as compared to its inhibition of a cysteine catalysis-based protein tyrosine phosphatase or a FCP/SCP family protein tyrosine phosphatase at the same concentration of the compound. Likewise, a compound at a given concentration may inhibit an EYA tyrosine phosphatase with 0.01 fold, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or greater than 1000 fold as much inhibitory activity as compared to its inhibition of a cysteine catalysis-based protein tyrosine phosphatase or a FCP/SCP family protein tyrosine phosphatase at the same concentration of the compound. Similarly, the comparison may be made by comparing the IC₅₀ of a compound towards an EYA tyrosine phosphatase with the IC₅₀ of the same compound towards a cysteine catalysis-based protein tyrosine phosphatase or a FCP/SCP family protein tyrosine phosphatase. For example, a compound may inhibit an EYA tyrosine phosphatase with the IC₅₀ that is 2 fold, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or greater than 100 fold of the IC₅₀ of the compound towards a cysteine catalysis-based protein tyrosine phosphatase or a FCP/SCP family protein tyrosine phosphatase. In some embodiments, the inhibition of EYA tyrosine phosphatase is tested first, followed by testing the inhibition of a cysteine catalysis-based protein tyrosine phosphatase or a FCP/SCP family protein tyrosine phosphatase.

In some embodiments, the method further comprises selecting a compound based on a comparison of on the inhibition of EYA tyrosine phosphatase versus inhibition of a cysteine catalysis-based protein tyrosine phosphatase or a FCP/SCP family protein tyrosine phosphatase. Typically, the compound will be selected as a specific inhibitor of an EYA tyrosine phosphatase when it exhibits inhibition of EYA tyrosine phosphatase that shows greater selectivity compared to another class or family of protein tyrosine phosphatases. Thus, for example, in some embodiments, a compound may be selected as an EYA tyrosine phosphatase inhibitor if the IC₅₀ towards EYA tyrosine phosphatase that is 2 fold, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 fold lower than the IC₅₀ of that compound towards a cysteine catalysis-based protein tyrosine phosphatase or a FCP/SCP family protein tyrosine phosphatase. An example of such a comparison and selection is set forth in Table 1 in Examples below.

### Methods of Screening for Inhibition of Disease States.

Also presented herein is a method of evaluating a compound for treatment of pulmonary arterial hypertension, comprising contacting an EYA tyrosine phosphatase with a compound and evaluating the results. In some embodiments, the compound comprises a compound as disclosed herein. In certain embodiments, the method comprises using an animal model for treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy. In certain embodiments, the method comprises using an animal model for preventing, reducing, or lowering the risk of vascular smooth muscle cell hyperproliferation, or preventing or reducing thickening of pulmonary artery walls.

Also presented herein is a method of evaluating a compound for inhibition of cell migration, proliferative retinopathy, retinopathy of prematurity, diabetic retinopathy, age related macular degeneration, retinal vasculitis, exudative vitreoretinopathy, tumor angiogenesis, hemangiomas or tumor metastasis, comprising contacting an EYA tyrosine phosphatase with a compound having the structure of Formulae I, II or III and evaluating the results.

Also presented herein is a method of evaluating a compound for inhibition of breast cancer (including ductal carcinoma lobule carcinoma and breast epithelial cancer), ovarian cancer (including epithelial ovarian cancer), desmoid tumor, malignant peripheral nerve sheath cancer, acute leukemia, rhabdomyosarcoma, Ewing's sarcoma, extra-skeletal myxoid chondrosarcoma, or endometrial cancer, comprising contacting an EYA tyrosine phosphatase with a compound having the structure of Formulae I, II or III and evaluating the results.

Also presented herein is a method of evaluating a compound for inhibition of Wilms' tumor, esophageal adenocarcinoma, colon cancer, colorectal cancer, esophageal squamous cell carcinoma, lung adenocarcinoma, Epstein-Barr virus-negative gastric cancer, or pancreatic ductal adenocarcinoma, comprising contacting an EYA tyrosine phosphatase with a compound having the structure of Formulae I, II or III and evaluating the results.

### Certain Pharmaceutical Agents

In certain embodiments, at least one compound as disclosed and described herein, or pharmaceutically acceptable salt, ester, amide, and/or prodrug thereof, either alone or combined with one or more pharmaceutically acceptable carriers, forms a pharmaceutical agent. Techniques for formulation and administration of compounds of the present embodiments may be found for example, in “Remington's Pharmaceutical Sciences,” Mack Publishing Co., Easton, Pa., 18th edition, 1990, which is incorporated herein by reference in its entirety.

In certain embodiments, a pharmaceutical agent comprising one or more compounds of the present embodiments is prepared using known techniques, including, but not limited to mixing, dissolving, granulating, dragee-making, levigating, emulsifying, encapsulating, entrapping or tabletting processes.

In certain embodiments, a pharmaceutical agent comprising one or more compounds of the present embodiments is a liquid (e.g., a suspension, elixir and/or solution). In certain of such embodiments, a liquid pharmaceutical agent comprising one or more compounds of the present embodiments is prepared using ingredients known in the art, including, but not limited to, water, glycols, oils, alcohols, flavoring agents, preservatives, and coloring agents.

In certain embodiments, a pharmaceutical agent comprising one or more compounds of the present embodiments is a solid (e.g., a powder, tablet, and/or capsule). In certain of such embodiments, a solid pharmaceutical agent comprising one or more compounds of the present embodiments is prepared using ingredients known in the art, including, but not limited to, starches, sugars, diluents, granulating agents, lubricants, binders, and disintegrating agents.

In certain embodiments, a pharmaceutical agent comprising one or more compounds of the present embodiments is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical agent comprising one or more compounds of the present embodiments comprises a delivery system. Examples of delivery systems include, but are not limited to, liposomes and emulsions. Certain delivery systems are useful for preparing certain pharmaceutical agents including those comprising hydrophobic compounds. In certain embodiments, certain organic solvents such as dimethylsulfoxide are used.

In certain embodiments, a pharmaceutical agent comprising one or more compounds of the present embodiments comprises one or more tissue-specific delivery molecules designed to deliver the pharmaceutical agent to specific tissues or cell types. For example, in certain embodiments, pharmaceutical agents include liposomes coated with a tissue-specific antibody.

In certain embodiments, a pharmaceutical agent comprising one or more compounds of the present embodiments comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™, and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol. e.g., polyvinyl pyrrolidone; and other sugars or polysaccharides may substitute for dextrose.

In certain embodiments, a pharmaceutical agent comprising one or more compounds of the present embodiments comprises a sustained-release system. A non-limiting example of such a sustained-release system is a semi-permeable matrix of solid hydrophobic polymers. In certain embodiments, sustained-release systems may, depending on their chemical nature, release compounds over a period of hours, days, weeks or months.

Certain compounds used in pharmaceutical agent of the present embodiments may be provided as pharmaceutically acceptable salts with pharmaceutically compatible counterions. Pharmaceutically compatible salts may be formed with many acids, including but not limited to hydrochloric, sulfuric, acetic, lactic, tartaric, malic, succinic, etc.

In certain embodiments, a pharmaceutical agent comprising one or more compounds of the present embodiments comprises an active ingredient in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, a pharmaceutical agent comprising one or more compounds of the present embodiments is useful for treating a conditions or disorder in a mammalian, and particularly in a human patient. Suitable administration routes include, but are not limited to, oral, rectal, transmucosal, intestinal, enteral, topical, suppository, through inhalation, intrathecal, intraventricular, intraperitoneal, intranasal, intraocular and parenteral (e.g., intravenous, intramuscular, intramedullary, and subcutaneous). In certain embodiments, pharmaceutical intrathecals are administered to achieve local rather than systemic exposures. For example, pharmaceutical agents may be injected directly in the area of desired effect (e.g., in the renal or cardiac area).

In certain embodiments, a pharmaceutical agent comprising one or more compounds of the present embodiments is administered in the form of a dosage unit (e.g., tablet, capsule, bolus, etc.). In certain embodiments, such dosage units comprise a compound as disclosed and described herein in a dose from about 1 μg/kg of body weight to about 50 mg/kg of body weight. In certain embodiments, such dosage units comprise a compound as disclosed and described herein in a dose from about 2 μg/kg of body weight to about 25 mg/kg of body weight. In certain embodiments, such dosage units comprise a compound as disclosed and described herein in a dose from about 10 μg/kg of body weight to about 5 mg/kg of body weight. In certain embodiments, pharmaceutical agents are administered as needed, once per day, twice per day, three times per day, or four or more times per day. It is recognized by those skilled in the art that the particular dose, frequency, and duration of administration depends on a number of factors, including, without limitation, the biological activity desired, the condition of the patient, and tolerance for the pharmaceutical agent.

In certain embodiments, a pharmaceutical agent comprising a compound of the present embodiments is prepared for oral administration. In certain of such embodiments, a pharmaceutical agent is formulated by combining one or more compounds of the present embodiments with one or more pharmaceutically acceptable carriers. Certain of such carriers enable compounds of the present embodiments to be formulated as tablets, pills, dragees, capsules, liquids, gels, syrups, slurries, suspensions and the like, for oral ingestion by a patient. In certain embodiments, pharmaceutical agents for oral use are obtained by mixing one or more compounds of the present embodiments and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical agents are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain of such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical agents for oral administration are push-fit capsules made of gelatin. Certain of such push-fit capsules comprise one or more compounds of the present embodiments in admixture with one or more filler such as lactose, binders such as starches, and/or lubricants such as talc or magnesium stearate and, optionally, stabilizers. In certain embodiments, pharmaceutical agents for oral administration are soft, sealed capsules made of gelatin and a plasticizer, such as glycerol or sorbitol. In certain soft capsules, one or more compounds of the present embodiments are be dissolved or suspended in suitable liquids, such as fatty oils, liquid paraffin, or liquid polyethylene glycols. In addition, stabilizers may be added.

In certain embodiments, pharmaceutical agents are prepared for buccal administration. Certain of such pharmaceutical agents are tablets or lozenges formulated in conventional manner.

In certain embodiments, a pharmaceutical agent is prepared for administration by injection (e.g., intravenous, subcutaneous, intramuscular, etc.). In certain of such embodiments, a pharmaceutical agent comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical agents for injection are presented in unit dosage form, e.g., in ampoules or in multi-dose containers. Certain pharmaceutical agents for injection are suspensions, solutions or emulsions in oily or aqueous vehicles, and may contain formulatory agents such as suspending, stabilizing and/or dispersing agents. Certain solvents suitable for use in pharmaceutical agents for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the compounds to allow for the preparation of highly concentrated solutions.

In certain embodiments, a pharmaceutical agent is prepared for transmucosal administration. In certain of such embodiments penetrants appropriate to the barrier to be permeated are used in the formulation. Such penetrants are generally known in the art.

In certain embodiments, a pharmaceutical agent is prepared for administration by inhalation. Certain of such pharmaceutical agents for inhalation are prepared in the form of an aerosol spray in a pressurized pack or a nebulizer. Certain of such pharmaceutical agents comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a compound of the present embodiments and a suitable powder base such as lactose or starch.

In certain embodiments, a pharmaceutical agent is prepared for rectal administration, such as a suppositories or retention enema. Certain of such pharmaceutical agents comprise known ingredients, such as cocoa butter and/or other glycerides.

In certain embodiments, a pharmaceutical agent is prepared for topical administration. Certain of such pharmaceutical agents comprise bland moisturizing bases, such as ointments or creams. Exemplary suitable ointment bases include, but are not limited to, petrolatum, petrolatum plus volatile silicones, lanolin and water in oil emulsions such as Eucerin™, available from Beiersdorf (Cincinnati, Ohio). Exemplary suitable cream bases include, but are not limited to, Nivea™ Cream, available from Beiersdorf (Cincinnati, Ohio), cold cream (USP), Purpose Cream™, available from Johnson & Johnson (New Brunswick. New Jersey), hydrophilic ointment (USP) and Lubriderm™, available from Pfizer (Morris Plains, N.J.).

In certain embodiments, the formulation, route of administration and dosage for a pharmaceutical agent of the present embodiments can be chosen in view of a particular patient's condition. (See e.g., Fingl et al. 1975, in “The Pharmacological Basis of Therapeutics”. Ch. 1 p. 1, which is incorporated herein by reference in its entirety). In certain embodiments, a pharmaceutical agent is administered as a single dose. In certain embodiments, a pharmaceutical agent is administered as a series of two or more doses administered over one or more days.

In certain embodiments, a pharmaceutical agent of the present embodiments is administered to a patient between about 0.1% and 500%, 5% and 200%, 10% and 100%, 15% and 85%, 25% and 75%, or 40% and 60% of an established human dosage. Where no human dosage is established, a suitable human dosage may be inferred from ED₅₀ or ID₅₀ values, or other appropriate values derived from in vitro or in vivo studies.

In certain embodiments, a daily dosage regimen for a patient comprises an oral dose of between 0.1 mg and 2000 mg, 5 mg and 1500 mg, 10 mg and 1000 mg, 20 mg and 500 mg, 30 mg and 200 mg, or 40 mg and 100 mg of a compound of the present embodiments. In certain embodiments, a daily dosage regimen is administered as a single daily dose. In certain embodiments, a daily dosage regimen is administered as two, three, four, or more than four doses. In certain embodiments, a daily dosage regimen for a patient for treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy patient comprises an oral dose of between 0.1 mg and 200 mg, 5 mg and 1500 mg, 10 mg and 1000 mg, 20 mg and 500 mg, 30 mg and 200 mg, or 40 mg and 100 mg of a compound of the present embodiments.

In certain embodiments, a pharmaceutical agent of the present embodiments is administered by continuous intravenous infusion. In certain of such embodiments, from 0.1 mg to 500 mg of a composition of the present embodiments is administered per day.

In certain embodiments, a pharmaceutical agent of the present embodiments is administered for a period of continuous therapy. For example, a pharmaceutical agent of the present embodiments may be administered over a period of days, weeks months, or years.

Dosage amount, interval between doses, and duration of treatment may be adjusted to achieve a desired effect. In certain embodiments, dosage amount and interval between doses are adjusted to maintain a desired concentration on compound in a patient. For example, in certain embodiments, dosage amount and interval between doses are adjusted to provide plasma concentration of a compound of the present embodiments at an amount sufficient to achieve a desired effect. In certain of such embodiments the plasma concentration is maintained above the minimal effective concentration (MEC). In certain embodiments, pharmaceutical agents of the present embodiments are administered with a dosage regimen designed to maintain a concentration above the MEC for 10-90% of the time, between 30-90% of the time, or between 50-90% of the time.

In certain embodiments in which a pharmaceutical agent is administered locally, the dosage regimen is adjusted to achieve a desired local concentration of a compound of the present embodiments.

In certain embodiments, a pharmaceutical agent may be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the present embodiments formulated in a compatible pharmaceutical carrier may also be prepared, placed in an appropriate container, and labeled for treatment of an indicated condition.

In certain embodiments, a pharmaceutical agent is in powder form for constitution with a suitable vehicle, e.g., sterile pyrogen-free water, before use.

### Methods of Treatment

Based on the teachings provided herein, the compounds provided herein can be used in methods of treating or relieving the symptoms of pulmonary arterial hypertension. Also, the compounds provided herein can be used in methods for treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy. In many embodiments, the compounds as described herein can be administered for a period of about 1 day to about 7 days, or about 1 week to about 2 weeks, or about 2 weeks to about 3 weeks or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The EYA tyrosine phosphatase inhibitor compounds as described herein can be administered 5 times per day, 4 times per day, 3 times per day or 2 times per day. In other embodiments, the EYA tyrosine phosphatase inhibitor compound is administered as a continuous infusion.

In many embodiments, a compounds as described herein of the embodiments can be administered orally or by inhalation.

In connection with the above-described methods for the treatment of pulmonary arterial hypertension in a patient, a compound as described herein may be administered to the patient at a dosage from about 0.01 mg to about 100 mg/kg patient bodyweight per day, in 1 to 5 divided doses per day. In some embodiments, the compounds as described herein can be administered at a dosage of about 0.5 mg to about 75 mg/kg patient bodyweight per day, in 1 to 5 divided doses per day. In connection with the above-described methods for treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy in a patient, a compound as described herein may be administered to the patient at a dosage from about 0.01 mg to about 100 mg/kg patient bodyweight per day, in 1 to 5 divided doses per day. In some embodiments, the compounds as described herein can be administered at a dosage of about 0.5 mg to about 75 mg/kg patient bodyweight per day, in 1 to 5 divided doses per day.

In many embodiments, a compound as described herein can be administered orally or by inhalation in an amount where the compound available to interact with EYA protein tyrosine phosphatase in a subject is at a level that inhibits EYA protein tyrosine phosphatase and produces a therapeutic effect, and the compound available to interact with protein tyrosine phosphatase-1b (PTP1B) and/or other cysteine-based protein tyrosine phosphatases is below levels known to be required to elicit a therapeutic effect meditated by the inhibition of protein tyrosine phosphatase-1b (PTP1B) and/or other cysteine-based protein tyrosine phosphatases.

In many embodiments, a compound is administered at a given amount or concentration that inhibits an EYA tyrosine phosphatase with 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 115%, 120%, 125%, 130%, 135%, 140%, 145%, 150%, 155%, 160%, 165%, 170%, 175%, 180%, 185%, 190%, 200%, 300%, 400% or greater than 500% as much inhibitory activity as compared to its inhibition of a cysteine catalysis-based protein tyrosine phosphatase or a FCP/SCP family protein tyrosine phosphatase at the same amount or concentration of the compound. Likewise, a compound administered at a given amount or concentration inhibits an EYA tyrosine phosphatase with 0.01 fold, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or greater than 1000 fold as much inhibitory activity as compared to its inhibition of a cysteine catalysis-based protein tyrosine phosphatase or a FCP/SCP family protein tyrosine phosphatase at the same amount or concentration of the compound. Similarly, the relative selectivity of an administered compound can be expressed by the IC₅₀ of the compound towards an EYA tyrosine phosphatase with the IC₅₀ of the same compound towards a cysteine catalysis-based protein tyrosine phosphatase or a FCP/SCP family protein tyrosine phosphatase. For example, an administered compound may inhibit an EYA tyrosine phosphatase with an IC₅₀ that is 2 fold, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or greater than 100 fold, of the IC₅₀ of the compound towards a cysteine catalysis-based protein tyrosine phosphatase or a FCP/SCP family protein tyrosine phosphatase.

In some embodiments, the method further comprises selecting a compound for administration based on a comparison of on the inhibition of EYA tyrosine phosphatase by the compound versus inhibition of a cysteine catalysis-based protein tyrosine phosphatase or a FCP/SCP family protein tyrosine phosphatase by the compound. Typically, the compound will be selected for administration as a specific inhibitor of an EYA tyrosine phosphatase when it exhibits inhibition of EYA tyrosine phosphatase that shows greater selectivity compared to another class or family of protein tyrosine phosphatases. Thus, for example, in some embodiments, a compound may be selected for administration as an EYA tyrosine phosphatase inhibitor if the IC₅₀ towards EYA tyrosine phosphatase that is 2 fold, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 fold lower than the IC₅₀ of that compound towards a cysteine catalysis-based protein tyrosine phosphatase or a FCP/SCP family protein tyrosine phosphatase. An example of such a comparison and selection is set forth in Table 1 in Examples below.

In some embodiments, based on the teachings provided herein, a compound having the structure of Formulae I, II or III can be used in methods of treating or relieving the symptoms of diseases such as proliferative retinopathy, retinopathy of prematurity, diabetic retinopathy, age related macular degeneration, retinal vasculitis, exudative vitreoretinopathy, tumor angiogenesis, hemangiomas or tumor metastasis. In many embodiments, the compounds as described herein can be administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The EYA tyrosine phosphatase inhibitor compounds as described herein can be administered 5 times per day, 4 times per day, 3 times per day or 2 times per day. In other embodiments, the EYA tyrosine phosphatase inhibitor compound is administered as a continuous infusion.

In some embodiments, based on the teachings provided herein, a compound as disclosed and described herein can be used in methods of treating or relieving the symptoms of pulmonary arterial hypertension. In some embodiments, based on the teachings provided herein, a compound as disclosed and described herein can be used in methods of treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy. In many embodiments, the compounds as described herein can be administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The EYA tyrosine phosphatase inhibitor compounds as described herein can be administered 5 times per day, 4 times per day, 3 times per day or 2 times per day. In other embodiments, the EYA tyrosine phosphatase inhibitor compound is administered as a continuous infusion.

In many embodiments, an compounds as described herein of the embodiments can be administered orally.

In connection with the above-described methods for the treatment of proliferative retinopathy, retinopathy of prematurity, diabetic retinopathy, age related macular degeneration, retinal vasculitis, exudative vitreoretinopathy, tumor angiogenesis, hemangiomas or tumor metastasis in a patient, a compound having the structure of Formulae I, II or III may be administered to the patient at a dosage from about 0.01 mg to about 100 mg/kg patient bodyweight per day, in 1 to 5 divided doses per day. In some embodiments, the compounds as described herein can be administered at a dosage of about 0.5 mg to about 75 mg/kg patient bodyweight per day, in 1 to 5 divided doses per day.

In connection with the above-described methods for the treatment of pulmonary arterial hypertension, a compound as disclosed and described herein may be administered to the patient at a dosage from about 0.01 mg to about 100 mg/kg patient bodyweight per day, in 1 to 5 divided doses per day. In connection with the above-described methods for treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy, a compound as disclosed and described herein may be administered to the patient at a dosage from about 0.01 mg to about 100 mg/kg patient bodyweight per day, in 1 to 5 divided doses per day. In some embodiments, the compounds as described herein can be administered at a dosage of about 0.5 mg to about 75 mg/kg patient bodyweight per day, in 1 to 5 divided doses per day.

In some embodiments, based on the teachings provided herein, a compound as disclosed and described herein can be used in methods of treating or relieving the symptoms of treatment of pulmonary arterial hypertension. In some embodiments, based on the teachings provided herein, a compound as disclosed and described herein can be used in methods of treating or relieving the symptoms of treatment of pulmonary arterial hypertension. In some embodiments, based on the teachings provided herein, a compound as disclosed and described herein can be used in methods of treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy. In many embodiments, the compounds as described herein can be administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The EYA tyrosine phosphatase inhibitor compounds as described herein can be administered 5 times per day, 4 times per day, 3 times per day or 2 times per day. In other embodiments, the EYA tyrosine phosphatase inhibitor compound is administered as a continuous infusion. In some embodiments, based on the teachings provided herein, a compound as disclosed and described herein can be used in methods of treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy. In some embodiments, based on the teachings provided herein, a compound as disclosed and described herein can be used in methods of preventing, reducing, or lowering the risk of vascular smooth muscle cell hyperproliferation, or preventing or reducing thickening of pulmonary artery walls.

In some embodiments, based on the teachings provided herein, a compound having the structure of Formulae I, II or III can be used in methods of treating or relieving the symptoms of diseases such as breast cancer (including ductal carcinoma lobule carcinoma and breast epithelial cancer), ovarian cancer (including epithelial ovarian cancer), desmoid tumor, malignant peripheral nerve sheath cancer, acute leukemia, rhabdomyosarcoma, Ewing's sarcoma, extra-skeletal myxoid chondrosarcoma, or endometrial cancer. In many embodiments, the compounds as described herein can be administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The EYA tyrosine phosphatase inhibitor compounds as described herein can be administered 5 times per day, 4 times per day, 3 times per day or 2 times per day. In other embodiments, the EYA tyrosine phosphatase inhibitor compound is administered as a continuous infusion.

In many embodiments, a compounds as described herein of the embodiments can be administered orally.

In connection with the above-described methods for the treatment of breast cancer (including ductal carcinoma lobule carcinoma and breast epithelial cancer), ovarian cancer (including epithelial ovarian cancer), desmoid tumor, malignant peripheral nerve sheath cancer, acute leukemia, rhabdomyosarcoma. Ewing's sarcoma, extra-skeletal myxoid chondrosarcoma, or endometrial cancer in a patient, a compound having the structure of Formulae I, II or III may be administered to the patient at a dosage from about 0.01 mg to about 100 mg/kg patient bodyweight per day, in 1 to 5 divided doses per day. In some embodiments, the compounds as described herein can be administered at a dosage of about 0.5 mg to about 75 mg/kg patient bodyweight per day, in 1 to 5 divided doses per day.

In connection with the above-described methods for the treatment of pulmonary arterial hypertension, a compound as disclosed and described herein may be administered to the patient at a dosage from about 0.01 mg to about 100 mg/kg patient bodyweight per day, in 1 to 5 divided doses per day. In connection with the above-described methods for treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy, a compound as disclosed and described herein may be administered to the patient at a dosage from about 0.01 mg to about 100 mg/kg patient bodyweight per day, in 1 to 5 divided doses per day. In connection with the above-described methods for preventing, reducing, or lowering the risk of vascular smooth muscle cell hyperproliferation, or preventing or reducing thickening of pulmonary artery walls, a compound as disclosed and described herein may be administered to the patient at a dosage from about 0.01 mg to about 100 mg/kg patient bodyweight per day, in 1 to 5 divided doses per day.

In some embodiments, the compounds as described herein can be administered at a dosage of about 0.5 mg to about 75 mg/kg patient bodyweight per day, in 1 to 5 divided doses per day.

In some embodiments, based on the teachings provided herein, a compound having the structure of Formulae I, II or III can be used in methods of treating or relieving the symptoms of diseases such as Wilms' tumor, esophageal adenocarcinoma, colon cancer, colorectal cancer, esophageal squamous cell carcinoma, lung adenocarcinoma, Epstein-Barr virus-negative gastric cancer, or pancreatic ductal adenocarcinoma. In many embodiments, the compounds as described herein can be administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The EYA tyrosine phosphatase inhibitor compounds as described herein can be administered 5 times per day, 4 times per day, 3 times per day or 2 times per day. In other embodiments, the EYA tyrosine phosphatase inhibitor compound is administered as a continuous infusion.

In some embodiments, based on the teachings provided herein, a compound as disclosed and described herein can be used in methods of treating or relieving the symptoms of pulmonary arterial hypertension. In some embodiments, based on the teachings provided herein, a compound as disclosed and described herein can be used in methods of treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy. In some embodiments, based on the teachings provided herein, a compound as disclosed and described herein can be used in methods for preventing, reducing, or lowering the risk of vascular smooth muscle cell hyperproliferation, or preventing or reducing thickening of pulmonary artery walls. In many embodiments, the compounds as described herein can be administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The EYA tyrosine phosphatase inhibitor compounds as described herein can be administered 5 times per day, 4 times per day, 3 times per day or 2 times per day. In other embodiments, the EYA tyrosine phosphatase inhibitor compound is administered as a continuous infusion.

In many embodiments, compounds as described herein of the embodiments can be administered orally.

In connection with the above-described methods for the treatment of Wilms' tumor, esophageal adenocarcinoma, colon cancer, colorectal cancer, esophageal squamous cell carcinoma, lung adenocarcinoma, Epstein-Barr virus-negative gastric cancer, or pancreatic ductal adenocarcinoma in a patient, a compound having the structure of Formulae I, II or III may be administered to the patient at a dosage from about 0.01 mg to about 100 mg/kg patient bodyweight per day, in 1 to 5 divided doses per day. In some embodiments, the compounds as described herein can be administered at a dosage of about 0.5 mg to about 75 mg/kg patient bodyweight per day, in 1 to 5 divided doses per day.

In connection with the above-described methods for the treatment of pulmonary arterial hypertension in a patient, a compound as disclosed and described herein may be administered to the patient at a dosage from about 0.01 mg to about 100 mg/kg patient bodyweight per day, in 1 to 5 divided doses per day. In connection with the above-described methods for treating or effecting pulmonary vascular remodeling, angio-obliterative pulmonary hypertension, idiopathic pulmonary arterial hypertension, right ventricular systolic pressure, right ventricular hypertrophy, vascular remodeling in distal pulmonary arterioles or neointimal arteriopathy, a compound as disclosed and described herein may be administered to the patient at a dosage from about 0.01 mg to about 100 mg/kg patient bodyweight per day, in 1 to 5 divided doses per day. In connection with the above-described methods for preventing, reducing, or lowering the risk of vascular smooth muscle cell hyperproliferation, or preventing or reducing thickening of pulmonary artery walls, a compound as disclosed and described herein may be administered to the patient at a dosage from about 0.01 mg to about 100 mg/kg patient bodyweight per day, in 1 to 5 divided doses per day. In some embodiments, the compounds as described herein can be administered at a dosage of about 0.5 mg to about 75 mg/kg patient bodyweight per day, in 1 to 5 divided doses per day.

The amount of active ingredient that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific compound, the severity of the symptoms and the susceptibility of the subject to side effects. Preferred dosages for a given compound as described herein can be readily determinable by those of skill in the art by a variety of means.

In certain embodiments, multiple doses of EYA tyrosine phosphatase inhibitor compound are administered. For example, an EYA tyrosine phosphatase inhibitor compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### EXAMPLES

The following examples are set forth merely to assist in understanding the embodiments and should not be construed as limiting the embodiments described and claimed herein in any way. Variations of the invention, including the substitution of all equivalents now known or later developed, which would be within the purview of those skilled in the art, and changes in formulation or minor changes in experimental design, are to be considered to fall within the scope of the embodiments incorporated herein.

The compounds of Formulae I, II, III and IV can be prepared according to methods known in the art. For example, the compounds of Formulae I, II, III and IV can be prepared according to the general method shown in Scheme 1 using the appropriate chemical reagents to obtain the desired compounds or the compounds of Formula II, can be prepared according to the general method shown in Scheme 2 using the appropriate chemical reagents to obtain the desired compounds.

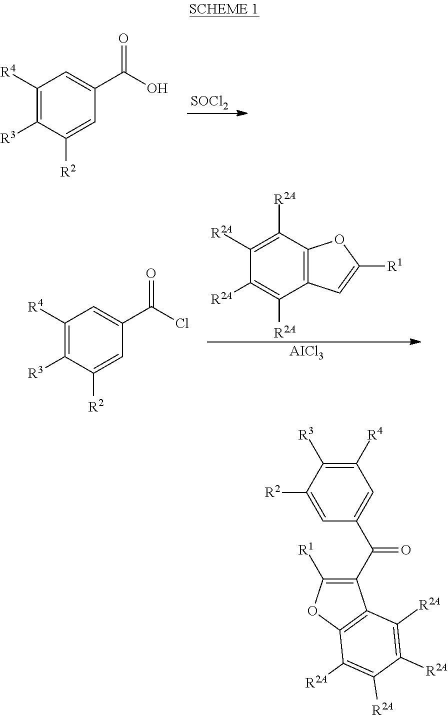

R¹-R⁴, and R^{2A} may be defined as disclosed for the compounds of Formulae I, II, III and IV described herein with appropriate selection in view of the synthetic protocol.

Examples of reaction conditions and specific synthetic procedures for the preparation of compounds of Formulae I, II, III and IV can be found in the methods described in Hu el al., “A Convergent Synthetic Study of Biologically Active Benzofuran Derivatives.” Arch Pharm Res, 2006, 29(6): 476478 and McDonald et al., “Warfarin-Amiodarone Drug-Drug Interactions: Determination of [I]ᵤ/K_{l,u} for Amiodarone and Its Plasma Metabolites,” Clin Pharmacol Ther, 2012, 91(4): 709-717 modified using the appropriate chemical reagents to obtain the desired compounds.

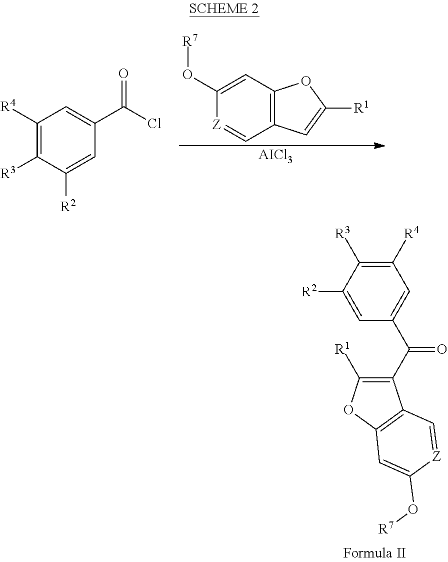

R¹-R⁴, R⁷ and Z may be defined as disclosed for the compounds of Formula H described herein with appropriate selection in view of the synthetic protocol.

### Example 1

### EYA-PTP Inhibition Reduces RV Systolic Pressure (RVSP) and RV Hypertrophy in a Rat Model of pH

The following experiment was performed to evaluate whether EYA-PTP inhibitor administration was effective when administered after PAH is typically established.

Sugen-Hypoxia (SuHx) rat model was used where a single administration of the VEGF inhibitor SU-5416 is followed by 3 weeks of hypoxia (10% O₂) and PAH develops between 3-5 weeks after rats (adult Sprague-Daley males) are returned to room air. To test the effect of EYA-PTP inhibition on the development of PAH, benzarone (BZ) was administered every 3 days for 5 weeks after return to room air. The rats were maintained for a further 3 weeks in room air and then right ventricular systolic pressure (RVSP) measured by right heart catheterization. The increase in RVSP in BZ-treated rats was 32% less in BZ-treated rats than in vehicle-treated rats. Fulton's index (right ventricle/left ventricle plus septum weight) was also lower (by 28% relative to vehicle-treated controls) in BZ-treated animals indicating reduced RV hypertrophy.

### Example 2

### EYA-PTP Inhibition Reduces DDR in Lungs from Rats Subject to the SUHX Protocol

The following experiment was performed to evaluate whether EYA-PTP inhibitor administration was effective in reducing DDR in lungs from rats subject to the SuHx protocol.

γ-H2AX foci report on the existence of DSBs, and the assembly of functional DDR complexes is characterized by the presence of 53BP1 foci. Rats subject to the SuHx protocol show increased γ-H2AX and 53BP1 staining in both PAEC and PASMC, likely as a result of oxidative stress. Rats treated with EYA-PTP inhibitor BZ had reduced γ-H2AX staining, as well as reduced 53BP1 foci formation in PASMC.

### Example 3

### Effect of EYA-PTP Inhibition or Genetic Loss of EYA3-PTP Activity on PASMC Cells from iPAH Patients

The following experiments are performed to evaluate the effect of EYA-PTP inhibition or genetic loss of EYA3-PTP activity on PASMC cells from iPAH patients.

Human PASMC over-expressing either EYA3 or the phosphatase-dead form EYA3(D262N), PAH-PASMC with CRISPR-generated knock-in of EYA3(D262N). PASMC cells isolated from control mice and from transgenic mice with Eya3 deletion or EYA3(D262N) knock-in demonstrates PAH-PASMC survival using gain- and loss-of-function approaches to determine the contribution of PTP activity of EYA3 to pro-proliferative and anti-apoptotic phenotype of PAH-PASMC.

Cells listed above are cultured in serum-free medium under either hypoxic (2.5% O₂, 5% CO₂) or standard (21% O₂, 5% CO₂) conditions and then monitored for survival (cell-counting kit CCK-8 and BrdU incorporation) and apoptosis (TUNEL assay, activation of caspase 9, efflux of cytochrome c, staining for annexin). Western blots are used to determine whether levels of EYA3 are altered by serum withdrawal and/or hypoxia. As controls we will probe for proteins known to be altered in PASMC by serum-withdrawal and hypoxia such as survivin63, RhoB64, and apoptotic mediators.

Cells listed above are subject to three DNA damaging protocols: etoposide. H₂O₂, γ-irradiation. Cell survival is measured; cell-counting kit CCK-8 and BrdU incorporation and TUNEL assay, activation of caspase 9, efflux of cytochrome c, and staining for annexin to measure apoptosis.

Cells listed above are used to query the effect of the EYA-PTP activity on H2AX-pY142 dephosphorylation where loss of EYA-PTP activity will result in increased levels of H2AX-pY142 hence preferential recruitment of JNK1 (rather than MDC1) to DNA damage foci, impaired DDR and increased apoptosis.

These results demonstrate that compound 1 specifically inhibits the phosphatase activity of Eya3.

### Example 4

### Correlation of Loss of EYA3-PTP Activity in Protecting Mice from Developing pH in the Chronic Hypoxia Model

The following experiments are performed to show correlation of loss of EYA3-PTP activity in protecting mice from developing PH in the chronic hypoxia model.

Global and tissue-specific loss of EYA3-PTP activity is measured. To obtain exclusive expression of Eya3(D262N) in either SMC or ECs Eya3^{DN} knock-in mice, are crossed with Eya3^{fl/fl};SMHC-cre/ER^{T2} or Eya3^{fl/fl};PdgJb-icreER to obtain Eya3^{DN/fx} mice. Tamoxifen-induced deletion of Eya3 in one allele will yield Eya3^{SMC DN/-} or Eya3^{VEC DN/-} mice. Non-targeted cells retain wild-type EYA3 and are functionally normal. The SuHx and chronic hypoxia protocols described above will be used on control and test mice followed by cardio-pulmonary assessment. Hemodynamics and RV hypertrophy will be measured. Pulmonary vascular remodeling will be assessed by measuring muscularization and wall thickness of small PAs, SMC proliferation by staining with Ki67 and α-SMA, assembly of DDR foci monitored by staining for γ-H2AX, and cardiac remodeling evaluated by measuring cardiomyocyte hypertrophy and cardiomyocyte fiber diameters in the right ventricle.

### Example 5

### Effect of EYA-PTP Inhibition on DDR and Survival of PAH-PASMC Under Oxidative Stress, and on PVR in an Investigational Rat Model of Severe Anglo-Obliterative pH and Right Ventricular Failure

The following experiments are performed to evaluate the effect of EYA-PTP inhibitors on DDR and survival of PAH-PASMC under oxidative stress, and on PVR in an investigational rat model of severe angio-obliterative PH and right ventricular failure.

BZ effectively reduces RVSP and PV remodeling in the rat SuHx model of PH. Reduction of RVSP and PV remodeling in the rat SuHx model of PH of compounds disclosed herein is compared to BZ for these effects, and evaluation of in vivo efficacy of EYA-PTP inhibitors and with dose- and route-of-administration testing.

Compounds that have an IC₅₀ better than 8 μM in enzymatic assays will be used in cell-based assays to determine levels inhibition of hypoxia-induced increase in PAH-PASMC proliferation, determine inhibition of survival of PAH-PASMC subject to DNA damaging protocols, and determine alteration H2AX-Y142 phosphorylation status and the assembly of DDR foci in PAH-PASMC. Target-specific effects of EYA-PTP inhibitors are using PAH-PASMC with CRISPR-generated knock-in of phosphatase-dead EYA3(D262N), and PAH-PASMC with Eya3 deletion.

Compounds will be delivered i.p. at a dosing schedule based on their EC₅₀ values in vitro. Compounds will be dissolved in aqueous buffer or peanut oil as appropriate and vehicle used in control animals. EYA inhibitor treatment will be started 5 weeks after rats return to room air when disease is established, and will be continued for 3 weeks. At the end of the experiment rats will undergo right heart catheterization to measure RVSP, and then sacrificed for morphological and histological analyses. Fulton's index, number of occluded distal pulmonary arteries, proliferation and apoptosis in lung tissue, PA muscularization, markers of DNA damage and repair will all be assessed. The PARP inhibitor ABT-888 that has been previously used in the rat SuHX protocol to inhibit DDR and reduce PH2 will be used as a positive control in these studies.

Compounds will then be tested by oral gavage. Efficacy upon oral administration implies sufficient bio-availability.

### Example 6

### Evaluation of Inhaled Benzarone

The following experiments are performed to evaluate the efficacy, pharmokinetics and toxicity of inhaled Benzarone as a PAH therapeutic agent.

Rats will be anesthetized with isoflurane (4%), placed in a supine position on an inclined platform, and metered amounts of Benzarone suspension will be instilled into the trachea (proximal to the bifurcation) via a laryngoscope-guided syringe. After instillation rats will be ventilated mechanically for about 1 minute to distribute the bolus within the lung. Based on preliminary studies a dose-range of 0.15-1.5 mg (0.3 mL injection of 1-10 mM stock) will be initially tested, administered every 3 days, 24 hours after the first dose and 24 hours after the last of 10 doses efficacy, pharmacokinetics, and toxicity will be measured as described below.

Pharmacokinetics: Blood samples will be collected by cardiac puncture at several time intervals between 10 min and 12 h after administration. Control animals will be administered vehicle alone. Aliquots of plasma will be mixed with cold acetonitrile containing propranolol, vortexed, centrifuged and the supernatant subject to LC-MS/MS. Using a noncompartmental model to describe the plasma concentration-time profiles of the test compounds, maximum concentration (Cmax), time required to reach maximum concentration (Tmax), area under the concentration-time curve (AUC), half-life (t½), apparent volume of distribution (Vz/F), and clearance rate (CLz/F) will be determined.

Hepatotoxicity will be assessed using both liver histopathology and serum chemistry. For serum chemistry blood samples will be collected from the abdominal aorta under anesthesia. Serum chemical indicators to be measured include alanine aminotransferase (ALT), aspartate aminotransferase (AST), alkaline phosphatase (ALP), total bilirubin (TBIL), direct bilirubin (DBIL), and gamma-glutamyl transpeptidase (GGT). Histopathological analyses conducted by blinded pathologists will evaluate markers of drug-induced liver injury 5 including hepatocellular necrosis/degeneration, inflammatory cell infiltration, bile duct proliferation, fibrosis, microvesicular steatosis, neutrophils, and portal venopathy.

Measures of success: Reduction of PH and RSVP, no histopathological findings of hepatotoxicity, serum chemical markers. The goal is TI>10.

Test efficacy, pharmacokinetics and toxicity of inhaled BZ. Inhaled delivery of a BZ-based therapeutic for PAH provides: direct delivery to the lung, higher local drug concentration at a lower administered dose, and avoiding systemic adverse effects (including hepatotoxicity). Aerosolized prostacyclins have been approved for PAH. Furthermore, when combined with animal disease models and pharmacodynamic measurements, inhalation dosing provides information on the duration of action required to maintain the targeted efficacy which aids in dose translation to humans.

1. A compound having the structure of Formula I: 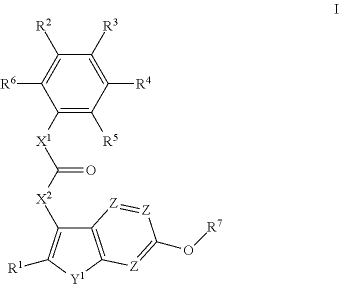
or a pharmaceutically acceptable salt thereof,
wherein:
R¹ is selected from the group consisting of C₁₋₆alkyl, C₂₋₆alkenyl, C₂₋₆alkynyl, aryl, heteroaryl, heterocyclyl, arylalkyl, heteroarylalkyl, heterocyclylalkyl, cycloalkyl, cycloalkenyl, (cyclolalkyl)alkyl, and amino, said C₁₋₆ alkyl, C₂₋₆ alkenyl, C₂₋₆ alkynyl, aryl, heteroaryl, heterocyclyl, arylalkyl, heteroarylalkyl, heterocyclylalkyl, cycloalkyl, cycloalkenyl, and (cyclolalkyl)alkyl are each optionally substituted with one or more R^{1A};
each R^{1A} is independently selected from the group consisting of hydroxy, halo, cyano, nitro, —C(═O)N(R^{1AA})₂, C₁₋₆alkyl optionally substituted with up to 5 fluoro, and C₁₋₆ alkoxy optionally substituted with up to 5 fluoro;
each R^{1AA} is independently selected from the group consisting of H (hydrogen), C₁₋₆ alkyl optionally substituted with up to 5 fluoro, and C₁₋₆alkyl substituted with one or more hydroxyl;
R² is selected from the group consisting of H (hydrogen), halo, hydroxy, and C₁₋₆ alkyl substituted with one or more hydroxy;
R³ is selected from the group consisting of halo, hydroxy, and C₁₋₆ alkyl substituted with one or more hydroxy;
R⁴ is H (hydrogen) or halo;
R⁵ and R⁶ are each independently selected from the group consisting of H (hydrogen), halo, cyano, C₁₋₆ alkyl, aryl, heteroaryl, heterocyclyl, and amino, said C₁₋₆ alkyl, aryl, heteroaryl, and heterocyclyl each optionally substituted with one or more R^{1A};
R⁷ is selected from the group consisting of —C(═O)aryl, —C(═O)C₁₋₆ alkyl and C₁₋₆ alkyl, said C₁₋₆ alkyl optionally substituted with one or more R^{1B};
each R^{1B} is independently selected from the group consisting of hydroxy, halo, aryl, heteroaryl, C₁₋₆ alkoxy optionally substituted with up to 5 fluoro;
X¹ is [C(R²)₂]ₙ, O (oxygen), or NR^{2A}, or X¹ is absent;
X² is [C(R^{2A})₂]ₙ, O (oxygen), or NR^{2A}, or X² is absent;
each R^{2A} is independently selected from the group consisting of H (hydrogen), halo, hydroxy, O-carbamyl, N-carbamyl, C-amido, S-sulfonamido, N-sulfonamido, C-carboxy, amino, aryl, heteroaryl, heterocyclyl, arylalkyl, heteroarylalkyl, heterocyclylalkyl, cycloalkyl, cycloalkenyl, (cyclolalkyl)alkyl, C₁₋₆ alkyl substituted with one or more hydroxyl, and C₁₋₆ alkyl optionally substituted with up to 5 fluoro;
each n is independently 1 or 2;
Y is O (oxygen), S (sulfur), or NR^{2A}; and
each Z is independently selected from the group consisting CR^{2A}, and N (nitrogen).

2. The compound of claim 1, wherein the compound having the structure of Formula I has the structure of Formula Ia, or Ib, 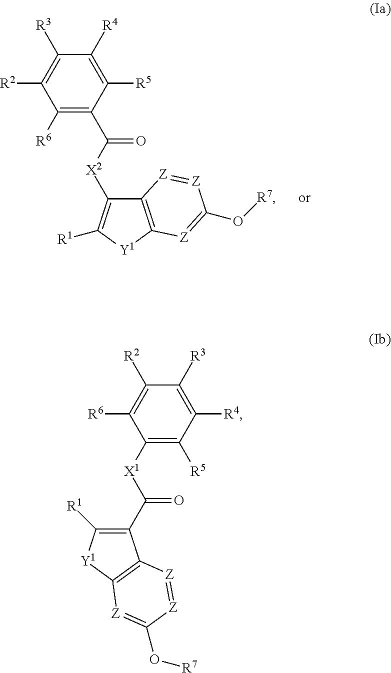
or a pharmaceutically acceptable salt thereof.

3. The compound of claim 2, wherein the compound having the structure of Formula I has the structure of Formula Ia, 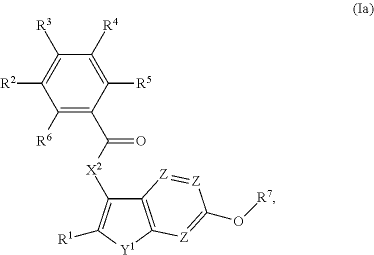
or a pharmaceutically acceptable salt thereof.

4. The compound of claim 2, wherein the compound having the structure of Formula I has the structure of Formula Ib, 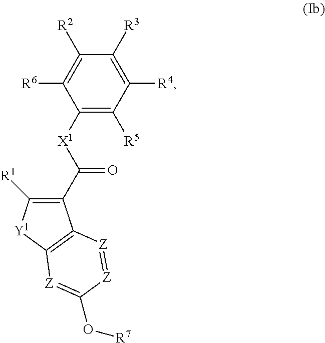
or a pharmaceutically acceptable salt thereof.

5. The compound of claim 1, wherein the compound of Formula I has the structure of Formula II: 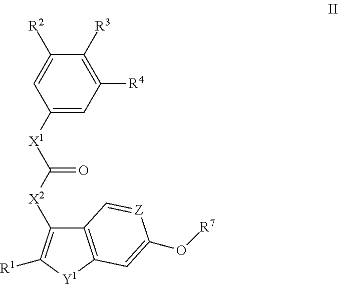
or a pharmaceutically acceptable salt thereof,
wherein:
X¹ is O (oxygen), or NR^{2A}, or X¹ is absent;
X² is O (oxygen), or NR^{2A}, or X² is absent;
each R² is independently selected from the group consisting of H (hydrogen), halo, hydroxy, C₁₋₆ alkyl substituted with one or more hydroxyl, and C₁₋₆ alkyl optionally substituted with up to 5 fluoro; and
Y is O (oxygen), or S (sulfur).

6. The compound of claim 5, wherein the compound having the structure of Formula II has the structure of Formula IIa, or IIb, 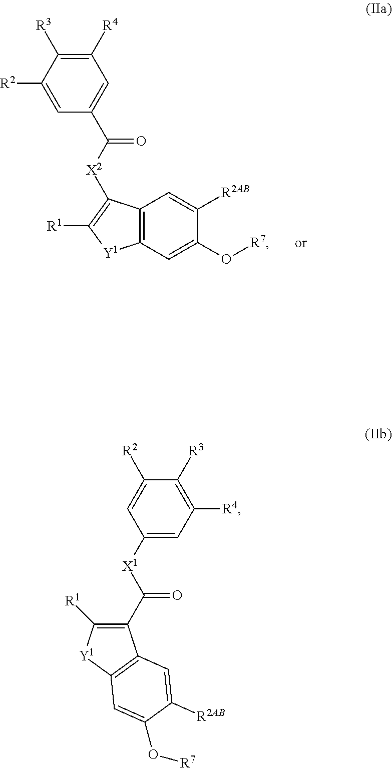
or a pharmaceutically acceptable salt thereof, wherein R^{2AB} is H (hydrogen) or hydroxyl.

7. The compound of claim 6, wherein the compound having the structure of Formula II has the structure of Formula IIa, 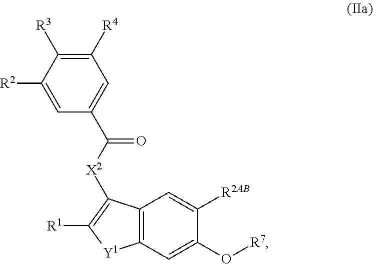
or a pharmaceutically acceptable salt thereof, wherein R^{2AB} is H (hydrogen) or hydroxyl.

8. The compound of claim 6, wherein the compound having the structure of Formula II has the structure of Formula IIb, 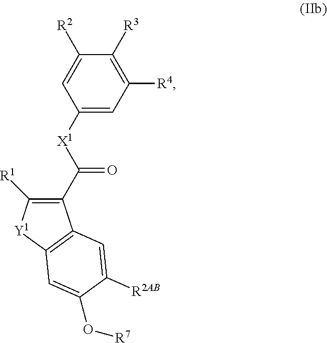
or a pharmaceutically acceptable salt thereof, wherein R^{2AB} is H (hydrogen) or hydroxyl.

9. The compound of claim 1, wherein the compound of Formula I has the structure of Formula III: 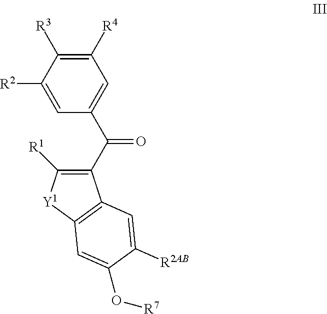
or a pharmaceutically acceptable salt thereof,
wherein:
R² is halo;
R³ is hydroxyl;
R⁴ is halo;
R^{2AB} is H (hydrogen) or hydroxyl; and
Y¹ is O (oxygen).

10. The compound of claim 9, wherein R² is iodo.

11. The compound of claim 9, wherein R^{2AB} is hydroxyl.

12. The compound of claim 1, wherein R¹ is C₁₋₆ alkyl optionally substituted with one or more R^{1A}.

13. The compound of claim 1, wherein R¹ is ethyl.

14. The compound of claim 1, wherein R² is iodo or bromo.

15. The compound of claim 1, wherein R⁴ is iodo or bromo.

16. A composition comprising a pharmaceutically acceptable excipient, and a compound of claim 1.

17. The compound of claim 1, wherein the compound is: 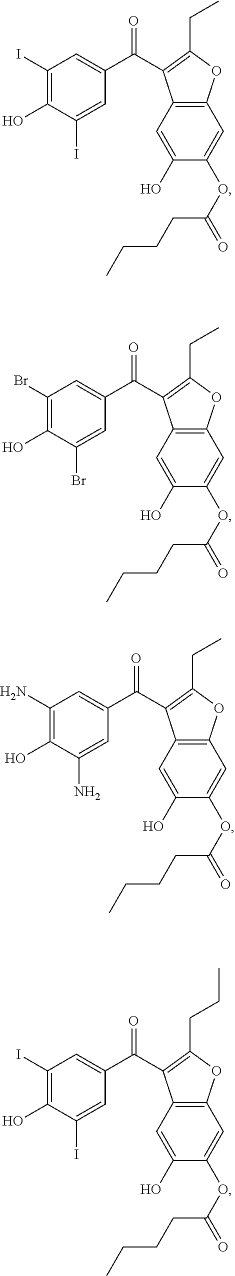 [SMILES: CCCCC(=O)Oc1cc2oc(CC)c(C(=O)c3cc(I)c(O)c(I)c3)c2cc1O ; CCCCC(=O)Oc1cc2oc(CC)c(C(=O)c3cc(Br)c(O)c(Br)c3)c2cc1O ; CCCCC(=O)Oc1cc2oc(CC)c(C(=O)c3cc(N)c(O)c(N)c3)c2cc1O ; CCCCC(=O)Oc1cc2oc(CCC)c(C(=O)c3cc(I)c(O)c(I)c3)c2cc1O] 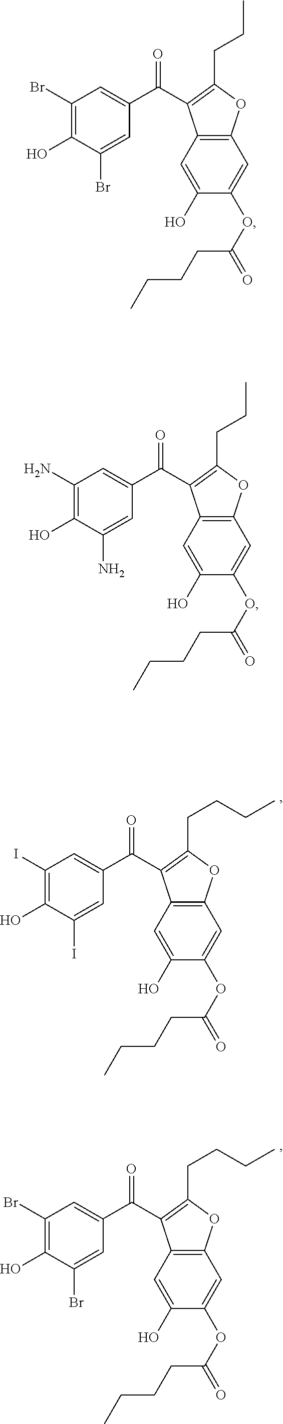 [SMILES: CCCCC(=O)Oc1cc2oc(CCC)c(C(=O)c3cc(Br)c(O)c(Br)c3)c2cc1O ; CCCCC(=O)Oc1cc2oc(CCC)c(C(=O)c3cc(N)c(O)c(N)c3)c2cc1O ; CCCCC(=O)Oc1cc2oc(CCCC)c(C(=O)c3cc(I)c(O)c(I)c3)c2cc1O ; CCCCC(=O)Oc1cc2oc(CCCC)c(C(=O)c3cc(Br)c(O)c(Br)c3)c2cc1O] 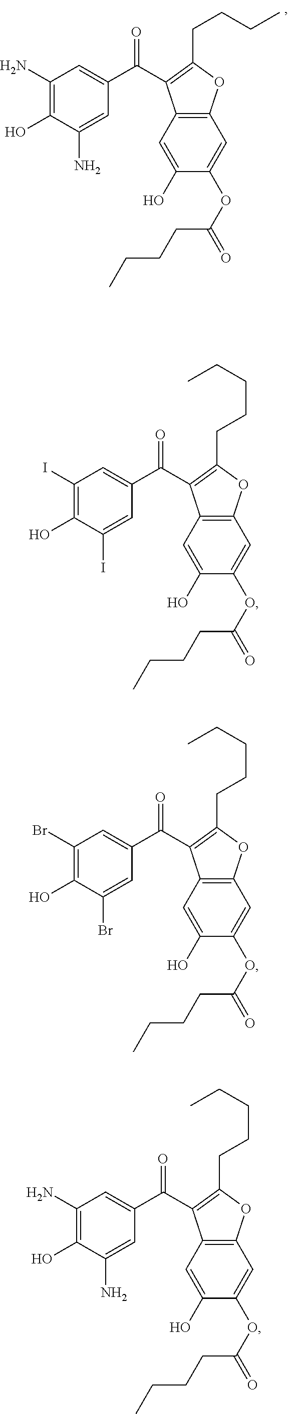 [SMILES: CCCCC(=O)Oc1cc2oc(CCCC)c(C(=O)c3cc(N)c(O)c(N)c3)c2cc1O ; CCCCCc1oc2cc(OC(=O)CCCC)c(O)cc2c1C(=O)c1cc(I)c(O)c(I)c1 ; CCCCCc1oc2cc(OC(=O)CCCC)c(O)cc2c1C(=O)c1cc(Br)c(O)c(Br)c1 ; CCCCCc1oc2cc(OC(=O)CCCC)c(O)cc2c1C(=O)c1cc(N)c(O)c(N)c1] 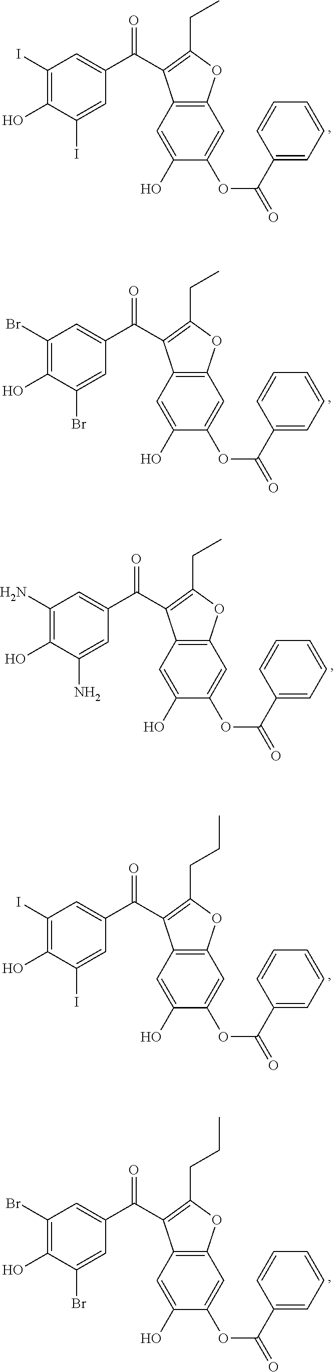 [SMILES: CCc1oc2cc(OC(=O)c3ccccc3)c(O)cc2c1C(=O)c1cc(I)c(O)c(I)c1 ; CCc1oc2cc(OC(=O)c3ccccc3)c(O)cc2c1C(=O)c1cc(Br)c(O)c(Br)c1 ; CCc1oc2cc(OC(=O)c3ccccc3)c(O)cc2c1C(=O)c1cc(N)c(O)c(N)c1 ; CCCc1oc2cc(OC(=O)c3ccccc3)c(O)cc2c1C(=O)c1cc(I)c(O)c(I)c1 ; CCCc1oc2cc(OC(=O)c3ccccc3)c(O)cc2c1C(=O)c1cc(Br)c(O)c(Br)c1] 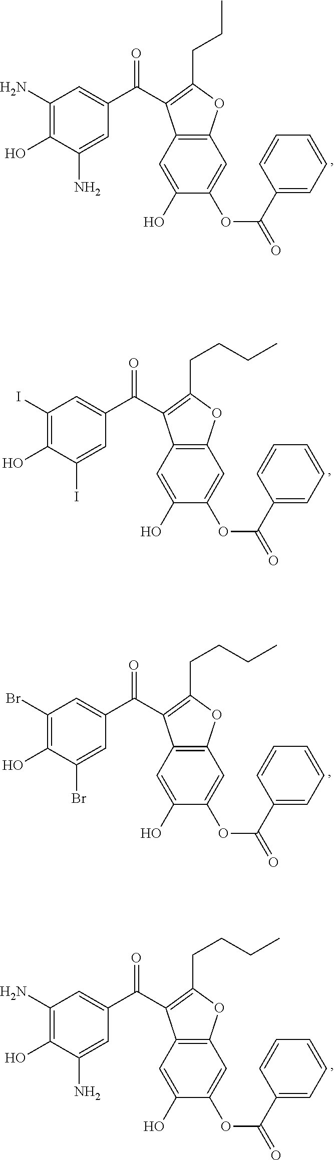 [SMILES: CCCc1oc2cc(OC(=O)c3ccccc3)c(O)cc2c1C(=O)c1cc(N)c(O)c(N)c1 ; CCCCc1oc2cc(OC(=O)c3ccccc3)c(O)cc2c1C(=O)c1cc(I)c(O)c(I)c1 ; CCCCc1oc2cc(OC(=O)c3ccccc3)c(O)cc2c1C(=O)c1cc(Br)c(O)c(Br)c1 ; CCCCc1oc2cc(OC(=O)c3ccccc3)c(O)cc2c1C(=O)c1cc(N)c(O)c(N)c1] 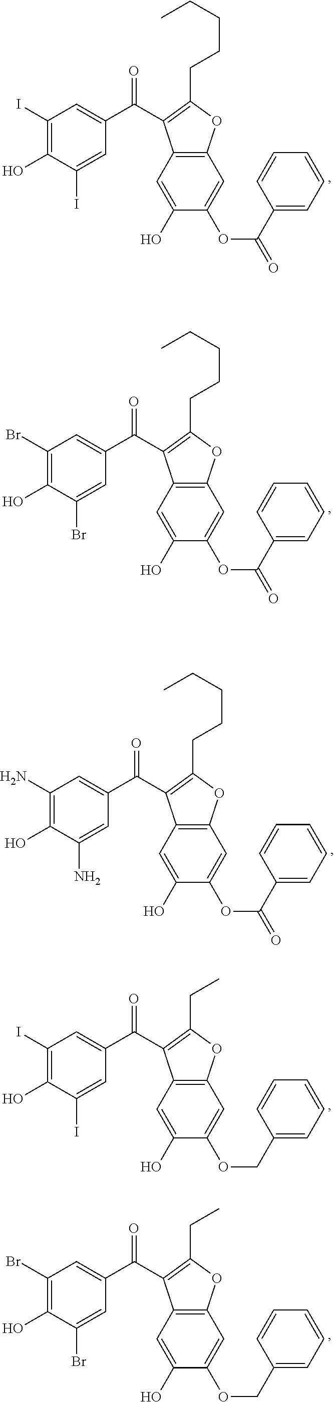 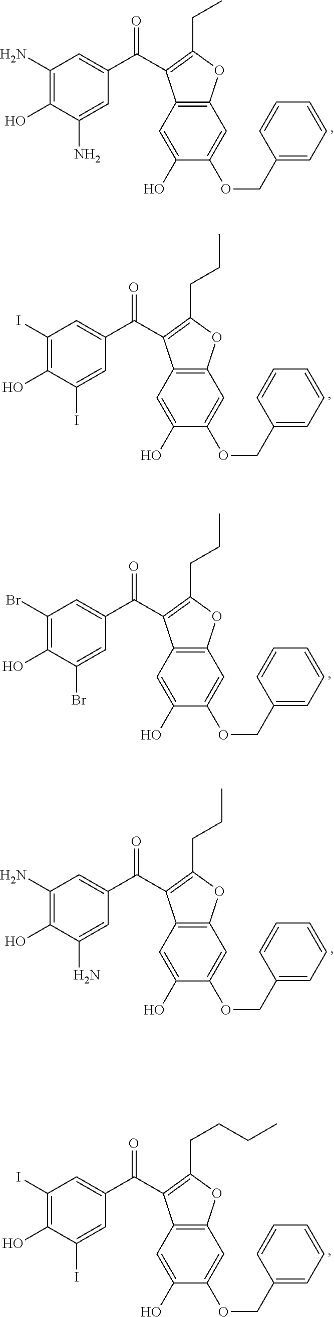 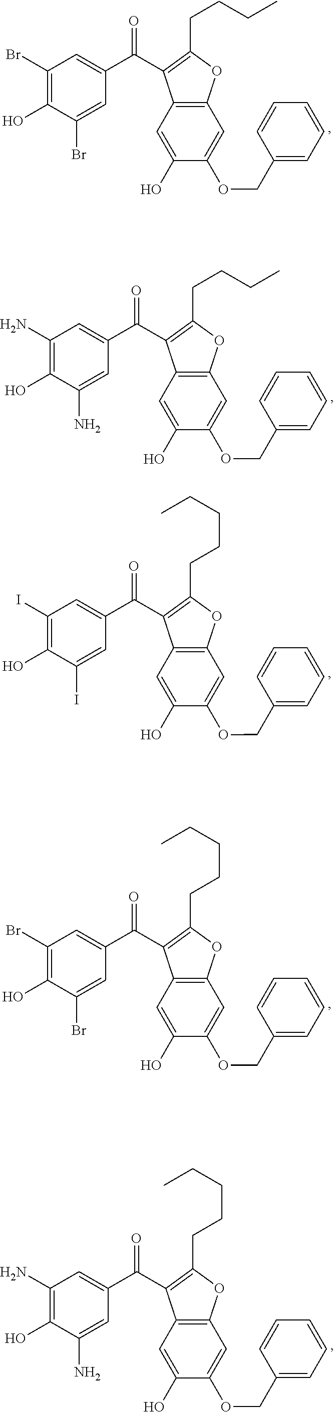 [SMILES: CCCCc1oc2cc(OCc3ccccc3)c(O)cc2c1C(=O)c1cc(Br)c(O)c(Br)c1 ; CCCCc1oc2cc(OCc3ccccc3)c(O)cc2c1C(=O)c1cc(N)c(O)c(N)c1 ; CCCCCc1oc2cc(OCc3ccccc3)c(O)cc2c1C(=O)c1cc(I)c(O)c(I)c1 ; CCCCCc1oc2cc(OCc3ccccc3)c(O)cc2c1C(=O)c1cc(Br)c(O)c(Br)c1 ; CCCCCc1oc2cc(OCc3ccccc3)c(O)cc2c1C(=O)c1cc(N)c(O)c(N)c1] 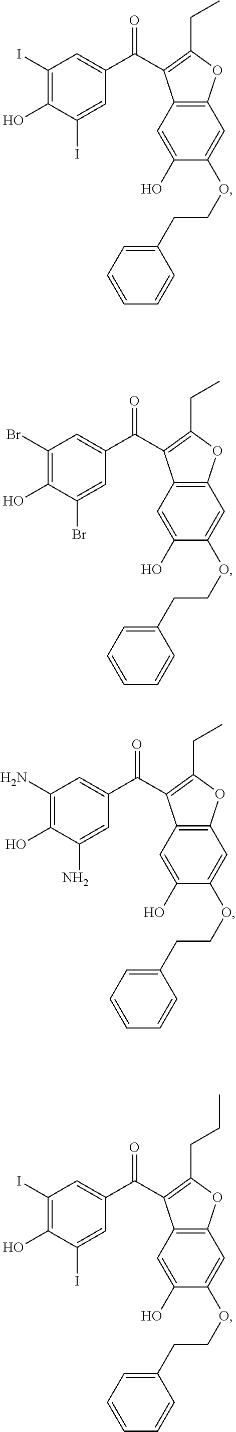 [SMILES: CCc1oc2cc(OCCc3ccccc3)c(O)cc2c1C(=O)c1cc(I)c(O)c(I)c1 ; CCc1oc2cc(OCCc3ccccc3)c(O)cc2c1C(=O)c1cc(Br)c(O)c(Br)c1 ; CCc1oc2cc(OCCc3ccccc3)c(O)cc2c1C(=O)c1cc(N)c(O)c(N)c1 ; CCCc1oc2cc(OCCc3ccccc3)c(O)cc2c1C(=O)c1cc(I)c(O)c(I)c1] 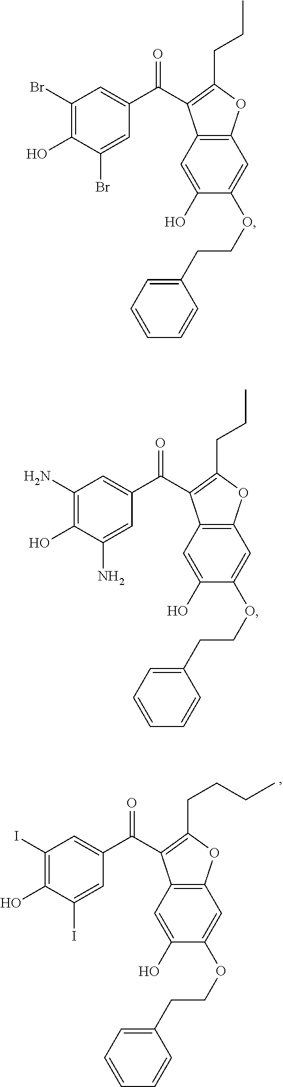 [SMILES: CCCc1oc2cc(OCCc3ccccc3)c(O)cc2c1C(=O)c1cc(Br)c(O)c(Br)c1 ; CCCc1oc2cc(OCCc3ccccc3)c(O)cc2c1C(=O)c1cc(N)c(O)c(N)c1 ; CCCCc1oc2cc(OCCc3ccccc3)c(O)cc2c1C(=O)c1cc(I)c(O)c(I)c1] 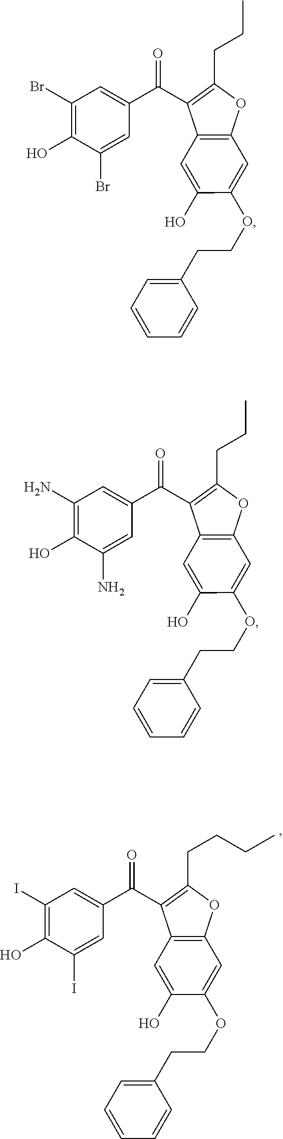 [SMILES: CCCc1oc2cc(OCCc3ccccc3)c(O)cc2c1C(=O)c1cc(Br)c(O)c(Br)c1 ; CCCc1oc2cc(OCCc3ccccc3)c(O)cc2c1C(=O)c1cc(N)c(O)c(N)c1 ; CCCCc1oc2cc(OCCc3ccccc3)c(O)cc2c1C(=O)c1cc(I)c(O)c(I)c1] 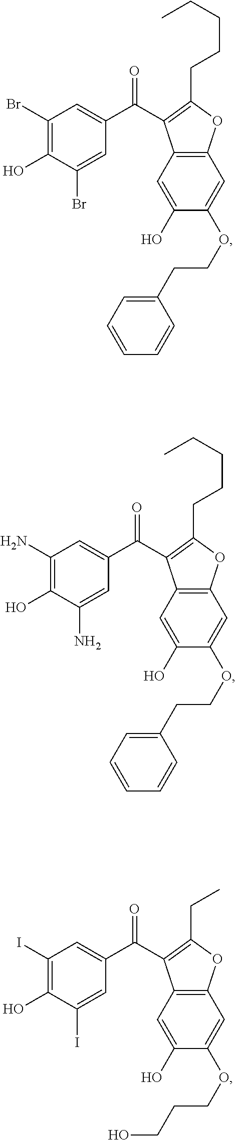 [SMILES: CCCCCc1oc2cc(OCCc3ccccc3)c(O)cc2c1C(=O)c1cc(Br)c(O)c(Br)c1 ; CCCCCc1oc2cc(OCCc3ccccc3)c(O)cc2c1C(=O)c1cc(N)c(O)c(N)c1 ; CCc1oc2cc(OCCCO)c(O)cc2c1C(=O)c1cc(I)c(O)c(I)c1] 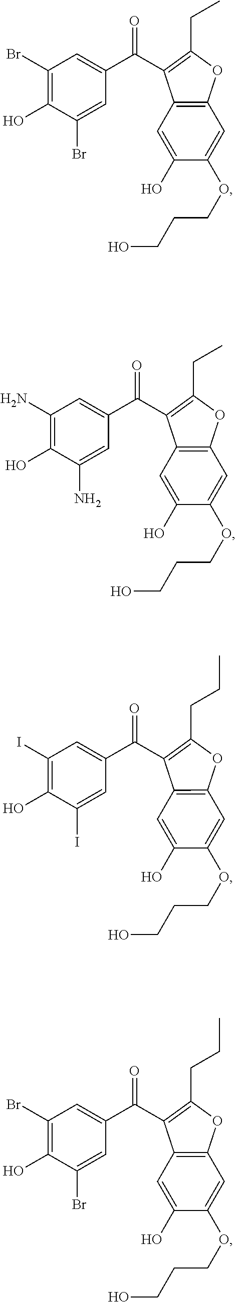 [SMILES: CCc1oc2cc(OCCCO)c(O)cc2c1C(=O)c1cc(Br)c(O)c(Br)c1 ; CCc1oc2cc(OCCCO)c(O)cc2c1C(=O)c1cc(N)c(O)c(N)c1 ; CCCc1oc2cc(OCCCO)c(O)cc2c1C(=O)c1cc(I)c(O)c(I)c1 ; CCCc1oc2cc(OCCCO)c(O)cc2c1C(=O)c1cc(Br)c(O)c(Br)c1] 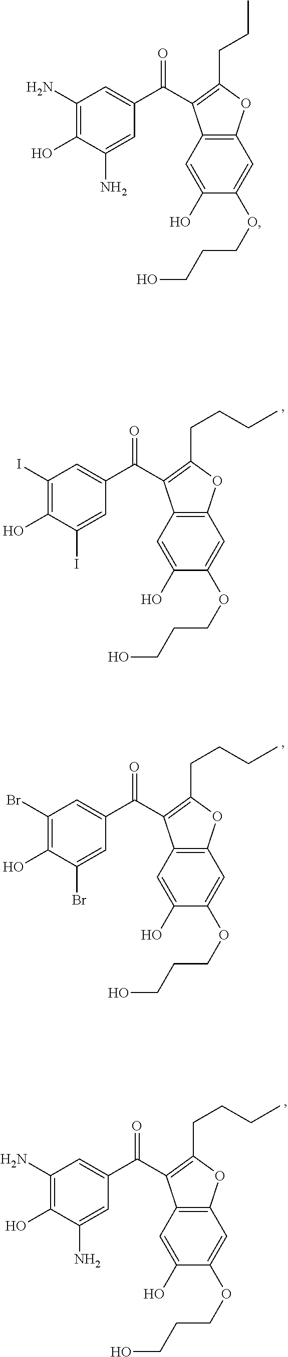 [SMILES: CCCc1oc2cc(OCCCO)c(O)cc2c1C(=O)c1cc(N)c(O)c(N)c1 ; CCCCc1oc2cc(OCCCO)c(O)cc2c1C(=O)c1cc(I)c(O)c(I)c1 ; CCCCc1oc2cc(OCCCO)c(O)cc2c1C(=O)c1cc(Br)c(O)c(Br)c1 ; CCCCc1oc2cc(OCCCO)c(O)cc2c1C(=O)c1cc(N)c(O)c(N)c1] 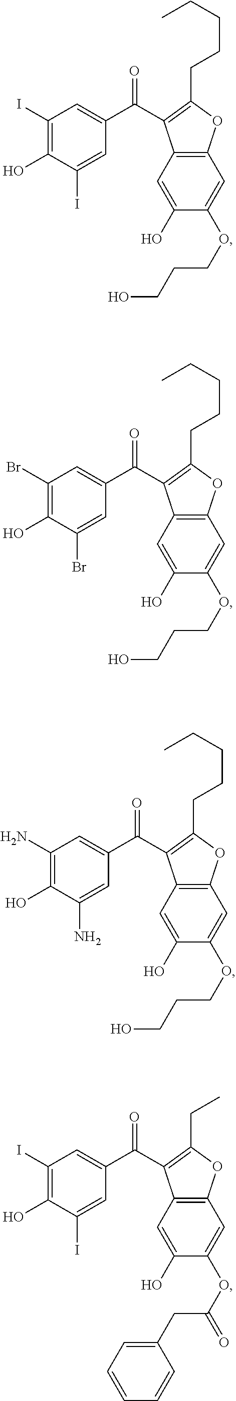 [SMILES: CCCCCc1oc2cc(OCCCO)c(O)cc2c1C(=O)c1cc(I)c(O)c(I)c1 ; CCCCCc1oc2cc(OCCCO)c(O)cc2c1C(=O)c1cc(Br)c(O)c(Br)c1 ; CCCCCc1oc2cc(OCCCO)c(O)cc2c1C(=O)c1cc(N)c(O)c(N)c1 ; CCc1oc2cc(OC(=O)Cc3ccccc3)c(O)cc2c1C(=O)c1cc(I)c(O)c(I)c1] 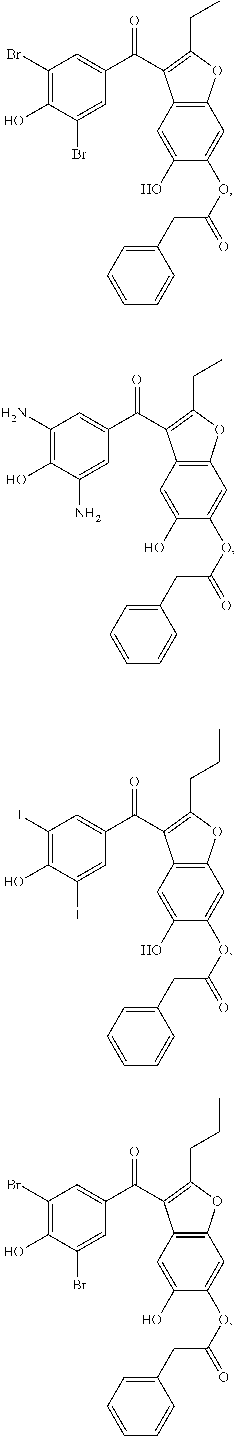 [SMILES: CCc1oc2cc(OC(=O)Cc3ccccc3)c(O)cc2c1C(=O)c1cc(Br)c(O)c(Br)c1 ; CCc1oc2cc(OC(=O)Cc3ccccc3)c(O)cc2c1C(=O)c1cc(N)c(O)c(N)c1 ; CCCc1oc2cc(OC(=O)Cc3ccccc3)c(O)cc2c1C(=O)c1cc(I)c(O)c(I)c1 ; CCCc1oc2cc(OC(=O)Cc3ccccc3)c(O)cc2c1C(=O)c1cc(Br)c(O)c(Br)c1] 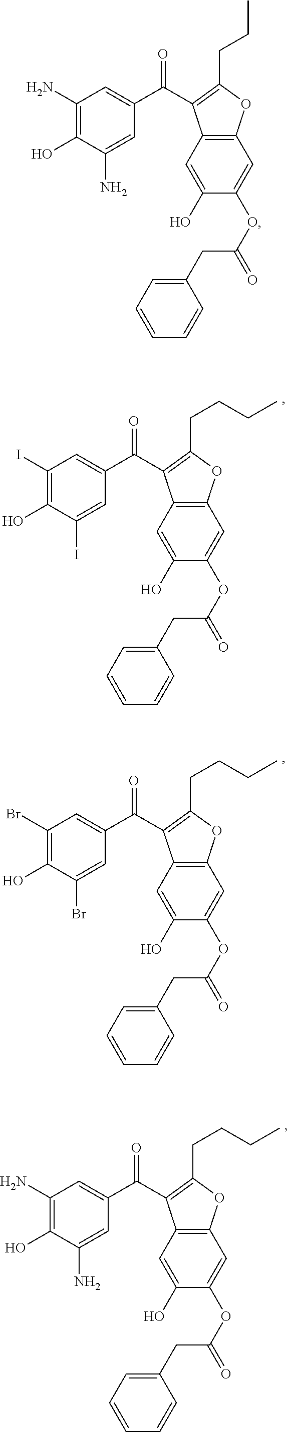 [SMILES: CCCc1oc2cc(OC(=O)Cc3ccccc3)c(O)cc2c1C(=O)c1cc(N)c(O)c(N)c1 ; CCCCc1oc2cc(OC(=O)Cc3ccccc3)c(O)cc2c1C(=O)c1cc(I)c(O)c(I)c1 ; CCCCc1oc2cc(OC(=O)Cc3ccccc3)c(O)cc2c1C(=O)c1cc(Br)c(O)c(Br)c1 ; CCCCc1oc2cc(OC(=O)Cc3ccccc3)c(O)cc2c1C(=O)c1cc(N)c(O)c(N)c1] 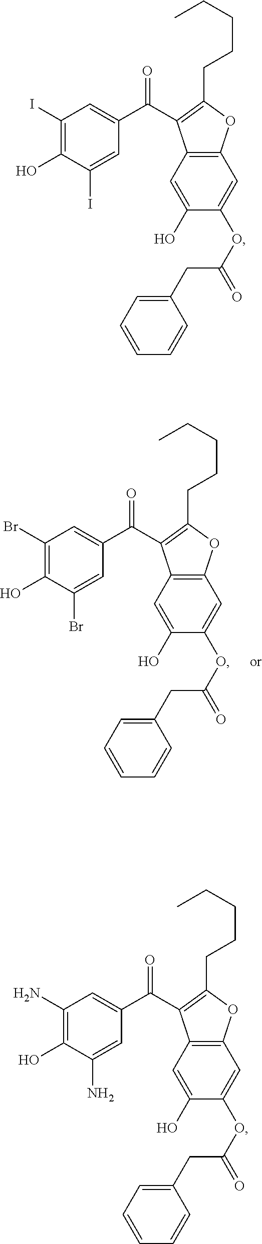 [SMILES: CCCCCc1oc2cc(OC(=O)Cc3ccccc3)c(O)cc2c1C(=O)c1cc(I)c(O)c(I)c1 ; CCCCCc1oc2cc(OC(=O)Cc3ccccc3)c(O)cc2c1C(=O)c1cc(Br)c(O)c(Br)c1 ; CCCCCc1oc2cc(OC(=O)Cc3ccccc3)c(O)cc2c1C(=O)c1cc(N)c(O)c(N)c1]
or a pharmaceutically acceptable salt thereof.

18. The compound of claim 1, wherein the compound is: 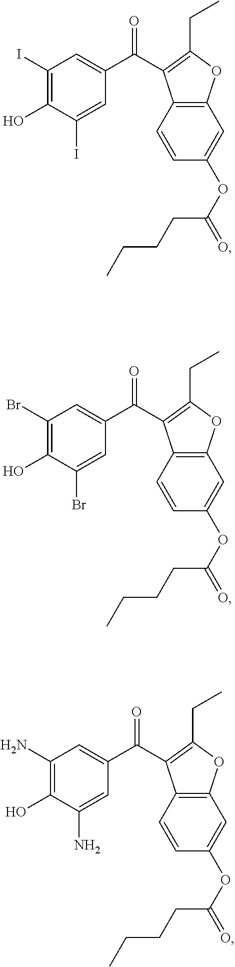 [SMILES: CCCCC(=O)Oc1ccc2c(C(=O)c3cc(I)c(O)c(I)c3)c(CC)oc2c1 ; CCCCC(=O)Oc1ccc2c(C(=O)c3cc(Br)c(O)c(Br)c3)c(CC)oc2c1 ; CCCCC(=O)Oc1ccc2c(C(=O)c3cc(N)c(O)c(N)c3)c(CC)oc2c1] 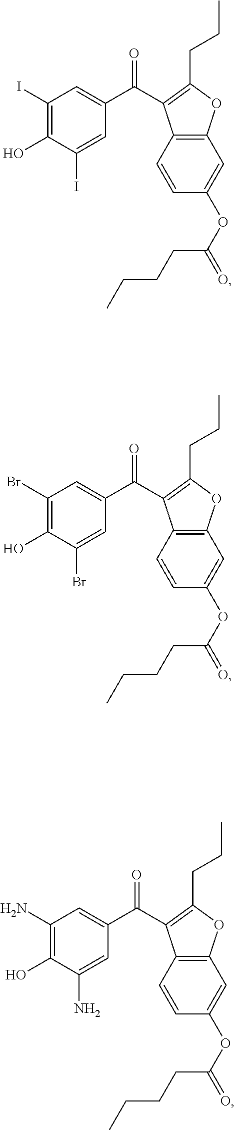 [SMILES: CCCCC(=O)Oc1ccc2c(C(=O)c3cc(I)c(O)c(I)c3)c(CCC)oc2c1 ; CCCCC(=O)Oc1ccc2c(C(=O)c3cc(Br)c(O)c(Br)c3)c(CCC)oc2c1 ; CCCCC(=O)Oc1ccc2c(C(=O)c3cc(N)c(O)c(N)c3)c(CCC)oc2c1] 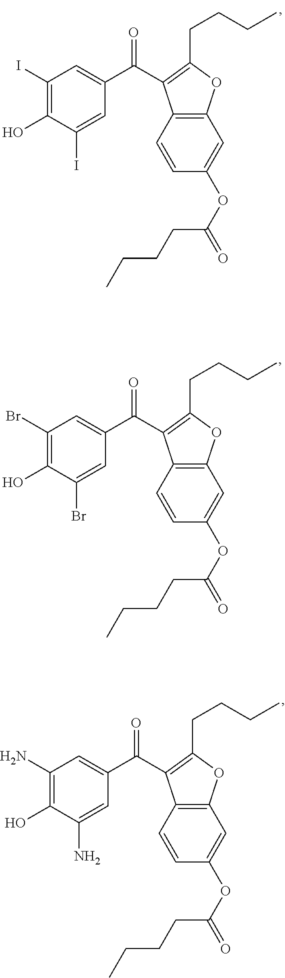 [SMILES: CCCCC(=O)Oc1ccc2c(C(=O)c3cc(I)c(O)c(I)c3)c(CCCC)oc2c1 ; CCCCC(=O)Oc1ccc2c(C(=O)c3cc(Br)c(O)c(Br)c3)c(CCCC)oc2c1 ; CCCCC(=O)Oc1ccc2c(C(=O)c3cc(N)c(O)c(N)c3)c(CCCC)oc2c1] 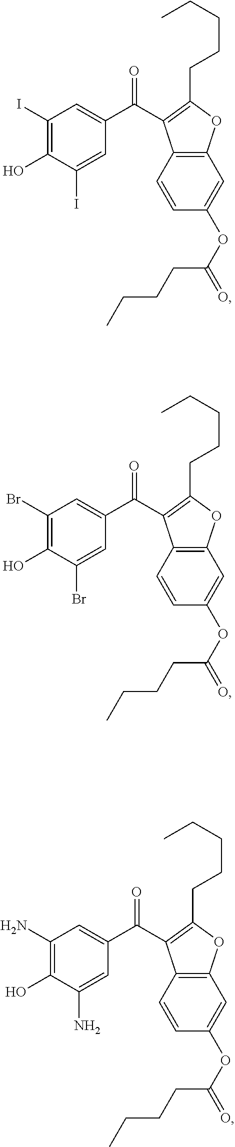 [SMILES: CCCCCc1oc2cc(OC(=O)CCCC)ccc2c1C(=O)c1cc(I)c(O)c(I)c1 ; CCCCCc1oc2cc(OC(=O)CCCC)ccc2c1C(=O)c1cc(Br)c(O)c(Br)c1 ; CCCCCc1oc2cc(OC(=O)CCCC)ccc2c1C(=O)c1cc(N)c(O)c(N)c1] 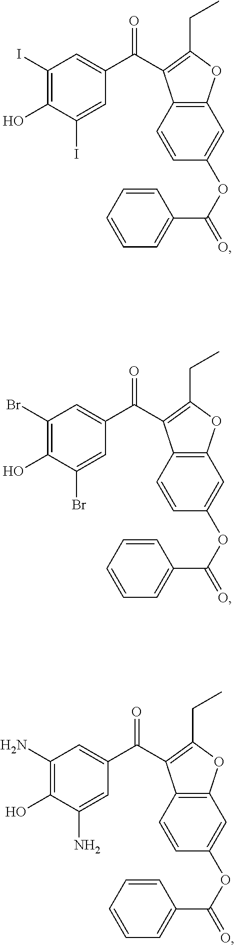 [SMILES: CCc1oc2cc(OC(=O)c3ccccc3)ccc2c1C(=O)c1cc(I)c(O)c(I)c1 ; CCc1oc2cc(OC(=O)c3ccccc3)ccc2c1C(=O)c1cc(Br)c(O)c(Br)c1 ; CCc1oc2cc(OC(=O)c3ccccc3)ccc2c1C(=O)c1cc(N)c(O)c(N)c1] 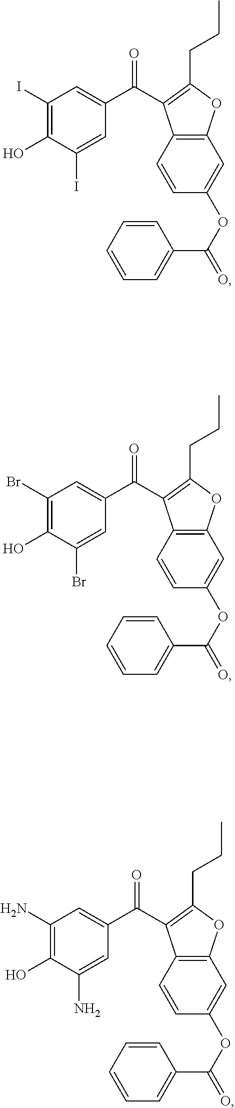 [SMILES: CCCc1oc2cc(OC(=O)c3ccccc3)ccc2c1C(=O)c1cc(I)c(O)c(I)c1 ; CCCc1oc2cc(OC(=O)c3ccccc3)ccc2c1C(=O)c1cc(Br)c(O)c(Br)c1 ; CCCc1oc2cc(OC(=O)c3ccccc3)ccc2c1C(=O)c1cc(N)c(O)c(N)c1] 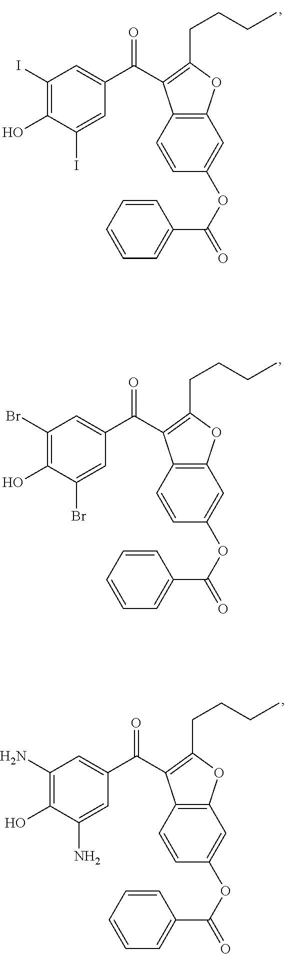 [SMILES: CCCCc1oc2cc(OC(=O)c3ccccc3)ccc2c1C(=O)c1cc(I)c(O)c(I)c1 ; CCCCc1oc2cc(OC(=O)c3ccccc3)ccc2c1C(=O)c1cc(Br)c(O)c(Br)c1 ; CCCCc1oc2cc(OC(=O)c3ccccc3)ccc2c1C(=O)c1cc(N)c(O)c(N)c1] 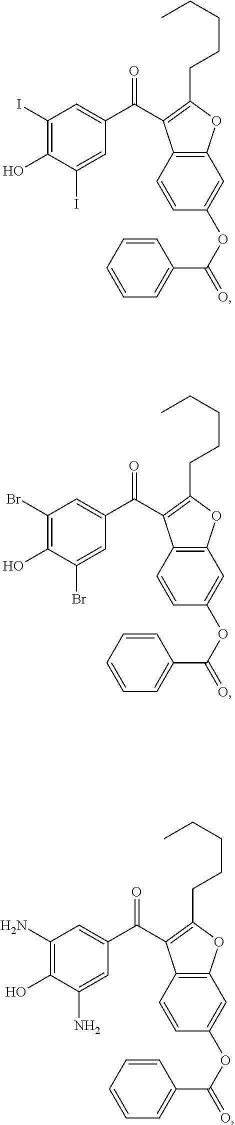 [SMILES: CCCCCc1oc2cc(OC(=O)c3ccccc3)ccc2c1C(=O)c1cc(I)c(O)c(I)c1 ; CCCCCc1oc2cc(OC(=O)c3ccccc3)ccc2c1C(=O)c1cc(Br)c(O)c(Br)c1 ; CCCCCc1oc2cc(OC(=O)c3ccccc3)ccc2c1C(=O)c1cc(N)c(O)c(N)c1] 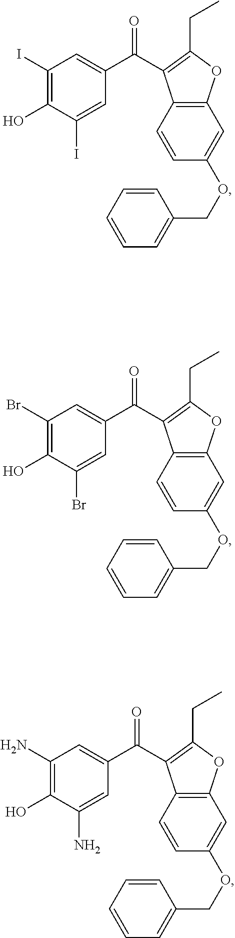 [SMILES: CCc1oc2cc(OCc3ccccc3)ccc2c1C(=O)c1cc(I)c(O)c(I)c1 ; CCc1oc2cc(OCc3ccccc3)ccc2c1C(=O)c1cc(Br)c(O)c(Br)c1 ; CCc1oc2cc(OCc3ccccc3)ccc2c1C(=O)c1cc(N)c(O)c(N)c1] 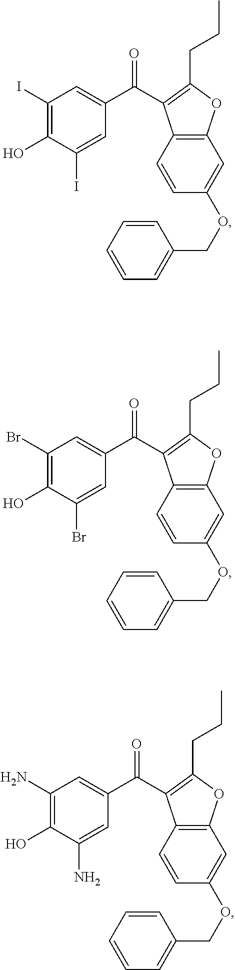 [SMILES: CCCc1oc2cc(OCc3ccccc3)ccc2c1C(=O)c1cc(I)c(O)c(I)c1 ; CCCc1oc2cc(OCc3ccccc3)ccc2c1C(=O)c1cc(Br)c(O)c(Br)c1 ; CCCc1oc2cc(OCc3ccccc3)ccc2c1C(=O)c1cc(N)c(O)c(N)c1] 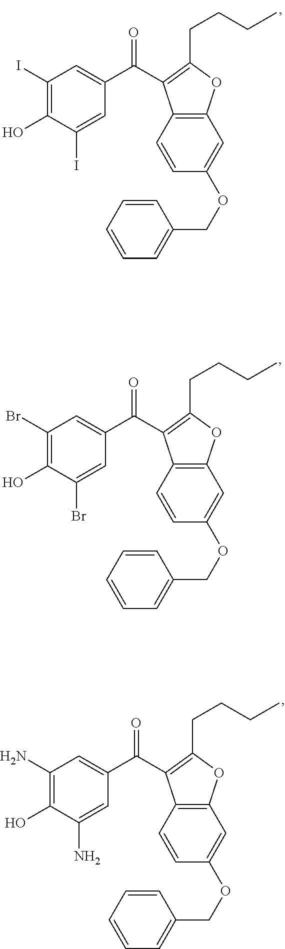 [SMILES: CCCCc1oc2cc(OCc3ccccc3)ccc2c1C(=O)c1cc(I)c(O)c(I)c1 ; CCCCc1oc2cc(OCc3ccccc3)ccc2c1C(=O)c1cc(Br)c(O)c(Br)c1 ; CCCCc1oc2cc(OCc3ccccc3)ccc2c1C(=O)c1cc(N)c(O)c(N)c1] 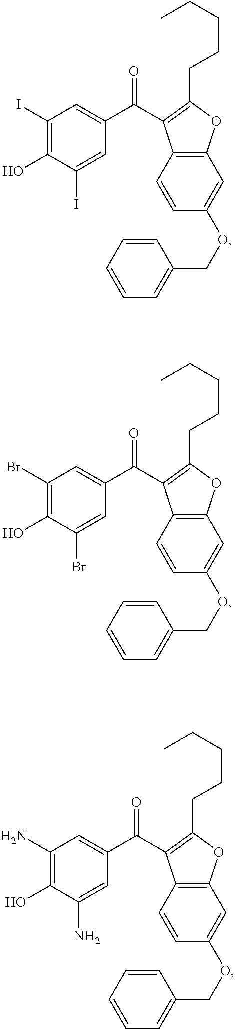 [SMILES: CCCCCc1oc2cc(OCc3ccccc3)ccc2c1C(=O)c1cc(I)c(O)c(I)c1 ; CCCCCc1oc2cc(OCc3ccccc3)ccc2c1C(=O)c1cc(Br)c(O)c(Br)c1 ; CCCCCc1oc2cc(OCc3ccccc3)ccc2c1C(=O)c1cc(N)c(O)c(N)c1] 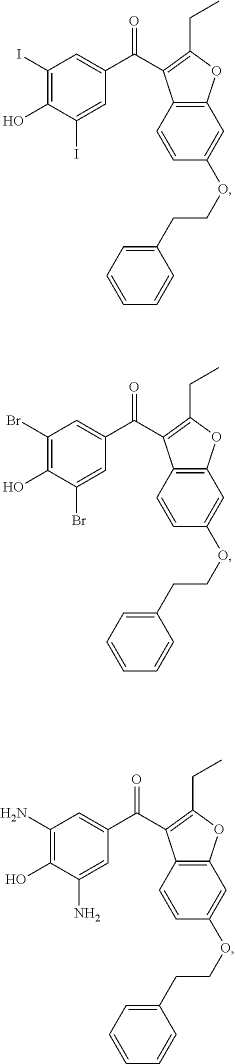 [SMILES: CCc1oc2cc(OCCc3ccccc3)ccc2c1C(=O)c1cc(I)c(O)c(I)c1 ; CCc1oc2cc(OCCc3ccccc3)ccc2c1C(=O)c1cc(Br)c(O)c(Br)c1 ; CCc1oc2cc(OCCc3ccccc3)ccc2c1C(=O)c1cc(N)c(O)c(N)c1] 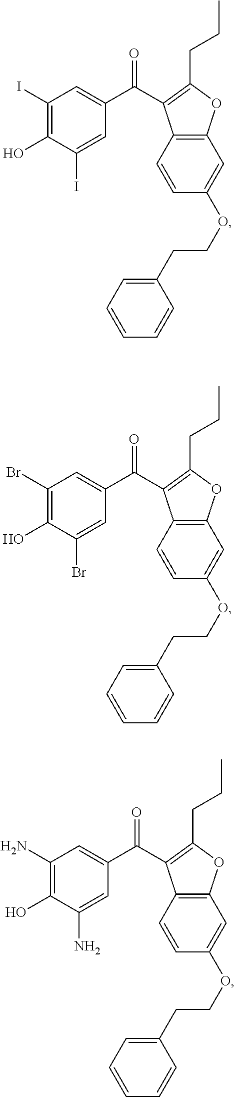 [SMILES: CCCc1oc2cc(OCCc3ccccc3)ccc2c1C(=O)c1cc(I)c(O)c(I)c1 ; CCCc1oc2cc(OCCc3ccccc3)ccc2c1C(=O)c1cc(Br)c(O)c(Br)c1 ; CCCc1oc2cc(OCCc3ccccc3)ccc2c1C(=O)c1cc(N)c(O)c(N)c1] 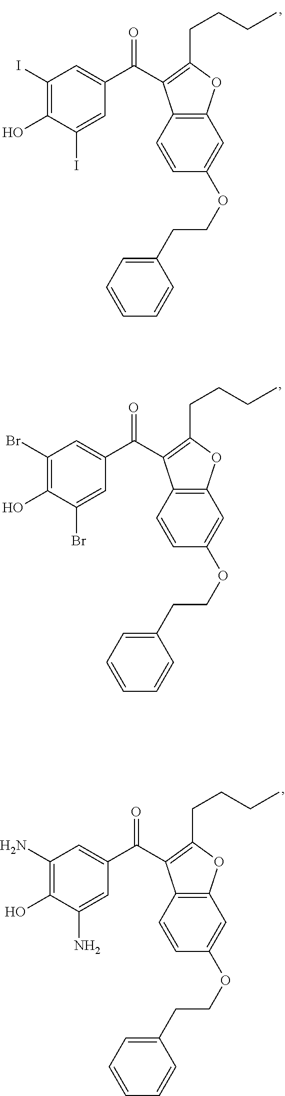 [SMILES: CCCCc1oc2cc(OCCc3ccccc3)ccc2c1C(=O)c1cc(I)c(O)c(I)c1 ; CCCCc1oc2cc(OCCc3ccccc3)ccc2c1C(=O)c1cc(Br)c(O)c(Br)c1 ; CCCCc1oc2cc(OCCc3ccccc3)ccc2c1C(=O)c1cc(N)c(O)c(N)c1] 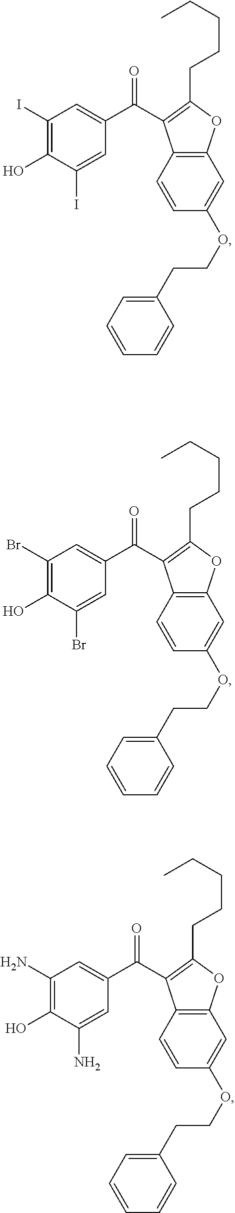 [SMILES: CCCCCc1oc2cc(OCCc3ccccc3)ccc2c1C(=O)c1cc(I)c(O)c(I)c1 ; CCCCCc1oc2cc(OCCc3ccccc3)ccc2c1C(=O)c1cc(Br)c(O)c(Br)c1 ; CCCCCc1oc2cc(OCCc3ccccc3)ccc2c1C(=O)c1cc(N)c(O)c(N)c1] 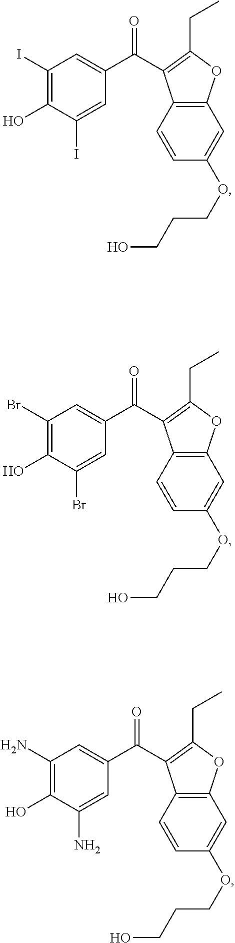 [SMILES: CCc1oc2cc(OCCCO)ccc2c1C(=O)c1cc(I)c(O)c(I)c1 ; CCc1oc2cc(OCCCO)ccc2c1C(=O)c1cc(Br)c(O)c(Br)c1 ; CCc1oc2cc(OCCCO)ccc2c1C(=O)c1cc(N)c(O)c(N)c1] 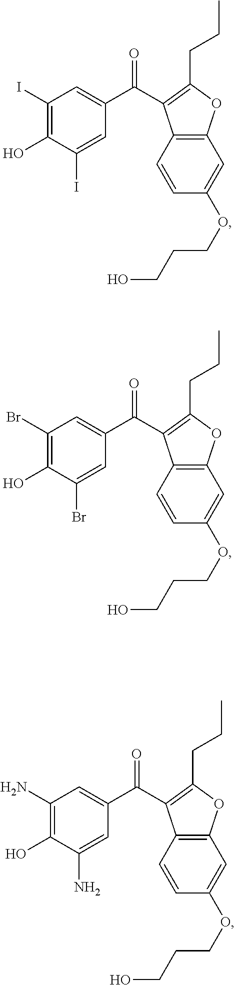 [SMILES: CCCc1oc2cc(OCCCO)ccc2c1C(=O)c1cc(I)c(O)c(I)c1 ; CCCc1oc2cc(OCCCO)ccc2c1C(=O)c1cc(Br)c(O)c(Br)c1 ; CCCc1oc2cc(OCCCO)ccc2c1C(=O)c1cc(N)c(O)c(N)c1] 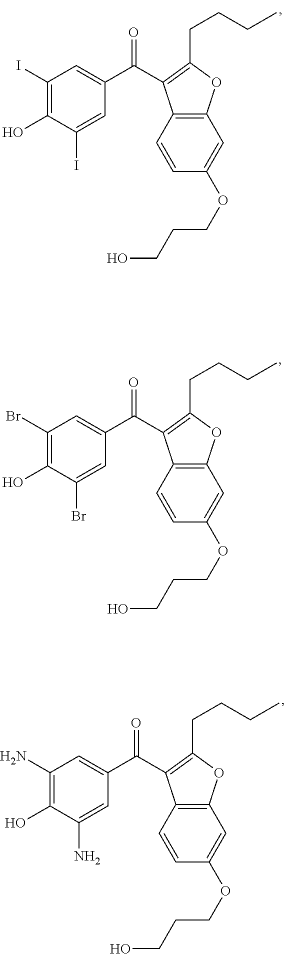 [SMILES: CCCCc1oc2cc(OCCCO)ccc2c1C(=O)c1cc(I)c(O)c(I)c1 ; CCCCc1oc2cc(OCCCO)ccc2c1C(=O)c1cc(Br)c(O)c(Br)c1 ; CCCCc1oc2cc(OCCCO)ccc2c1C(=O)c1cc(N)c(O)c(N)c1] 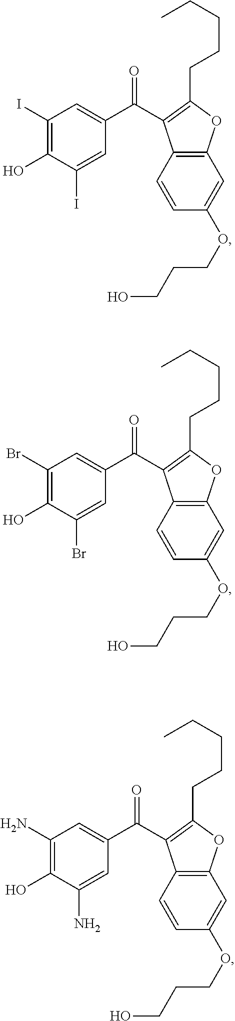 [SMILES: CCCCCc1oc2cc(OCCCO)ccc2c1C(=O)c1cc(I)c(O)c(I)c1 ; CCCCCc1oc2cc(OCCCO)ccc2c1C(=O)c1cc(Br)c(O)c(Br)c1 ; CCCCCc1oc2cc(OCCCO)ccc2c1C(=O)c1cc(N)c(O)c(N)c1] 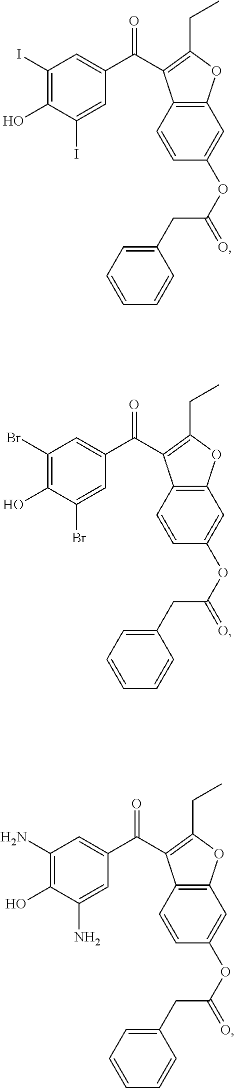 [SMILES: CCc1oc2cc(OC(=O)Cc3ccccc3)ccc2c1C(=O)c1cc(I)c(O)c(I)c1 ; CCc1oc2cc(OC(=O)Cc3ccccc3)ccc2c1C(=O)c1cc(Br)c(O)c(Br)c1 ; CCc1oc2cc(OC(=O)Cc3ccccc3)ccc2c1C(=O)c1cc(N)c(O)c(N)c1] 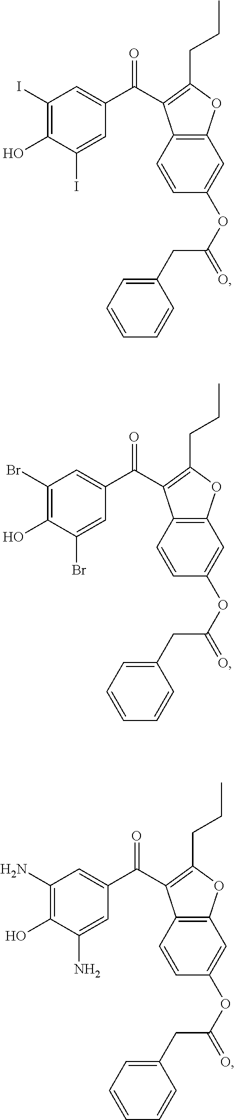 [SMILES: CCCc1oc2cc(OC(=O)Cc3ccccc3)ccc2c1C(=O)c1cc(I)c(O)c(I)c1 ; CCCc1oc2cc(OC(=O)Cc3ccccc3)ccc2c1C(=O)c1cc(Br)c(O)c(Br)c1 ; CCCc1oc2cc(OC(=O)Cc3ccccc3)ccc2c1C(=O)c1cc(N)c(O)c(N)c1] 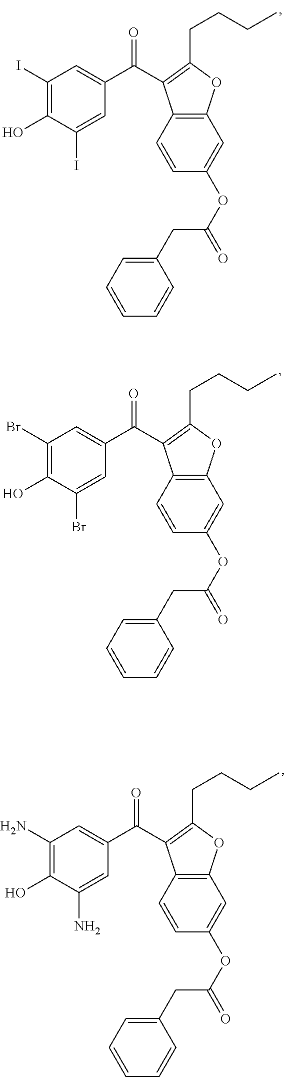 [SMILES: CCCCc1oc2cc(OC(=O)Cc3ccccc3)ccc2c1C(=O)c1cc(I)c(O)c(I)c1 ; CCCCc1oc2cc(OC(=O)Cc3ccccc3)ccc2c1C(=O)c1cc(Br)c(O)c(Br)c1 ; CCCCc1oc2cc(OC(=O)Cc3ccccc3)ccc2c1C(=O)c1cc(N)c(O)c(N)c1] 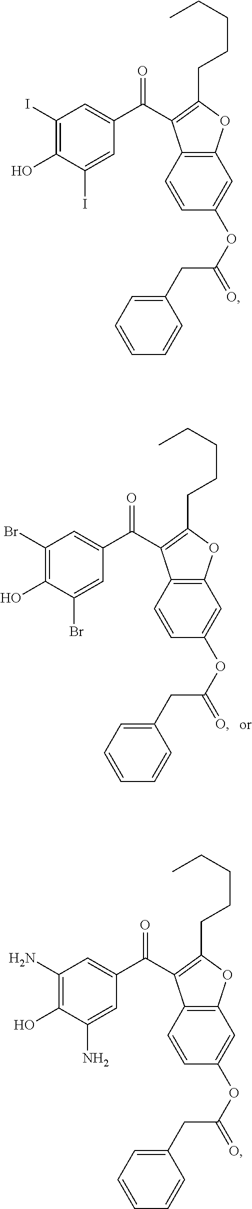 [SMILES: CCCCCc1oc2cc(OC(=O)Cc3ccccc3)ccc2c1C(=O)c1cc(I)c(O)c(I)c1 ; CCCCCc1oc2cc(OC(=O)Cc3ccccc3)ccc2c1C(=O)c1cc(Br)c(O)c(Br)c1 ; CCCCCc1oc2cc(OC(=O)Cc3ccccc3)ccc2c1C(=O)c1cc(N)c(O)c(N)c1]
or a pharmaceutically acceptable salt thereof.

19. The compound of claim 1, wherein the compound is: 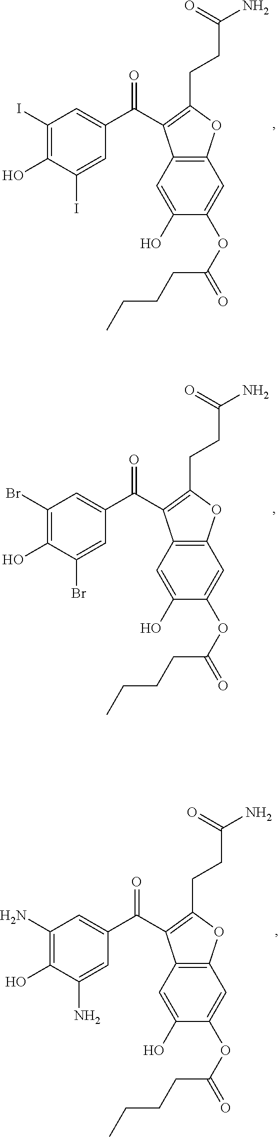 [SMILES: CCCCC(=O)Oc1cc2oc(CCC(N)=O)c(C(=O)c3cc(I)c(O)c(I)c3)c2cc1O ; CCCCC(=O)Oc1cc2oc(CCC(N)=O)c(C(=O)c3cc(Br)c(O)c(Br)c3)c2cc1O ; CCCCC(=O)Oc1cc2oc(CCC(N)=O)c(C(=O)c3cc(N)c(O)c(N)c3)c2cc1O] 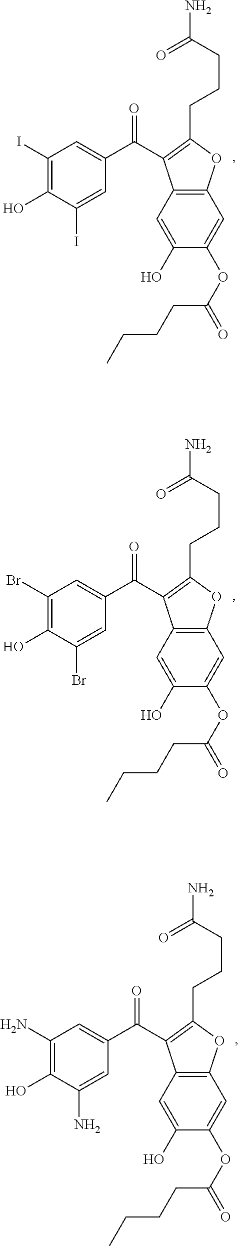 [SMILES: CCCCC(=O)Oc1cc2oc(CCCC(N)=O)c(C(=O)c3cc(I)c(O)c(I)c3)c2cc1O ; CCCCC(=O)Oc1cc2oc(CCCC(N)=O)c(C(=O)c3cc(Br)c(O)c(Br)c3)c2cc1O ; CCCCC(=O)Oc1cc2oc(CCCC(N)=O)c(C(=O)c3cc(N)c(O)c(N)c3)c2cc1O] 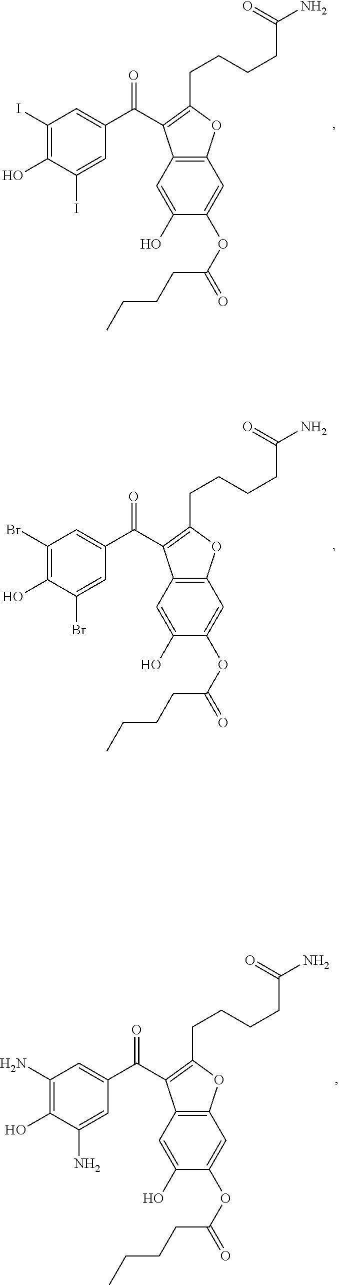 [SMILES: CCCCC(=O)Oc1cc2oc(CCCCC(N)=O)c(C(=O)c3cc(I)c(O)c(I)c3)c2cc1O ; CCCCC(=O)Oc1cc2oc(CCCCC(N)=O)c(C(=O)c3cc(Br)c(O)c(Br)c3)c2cc1O ; CCCCC(=O)Oc1cc2oc(CCCCC(N)=O)c(C(=O)c3cc(N)c(O)c(N)c3)c2cc1O] 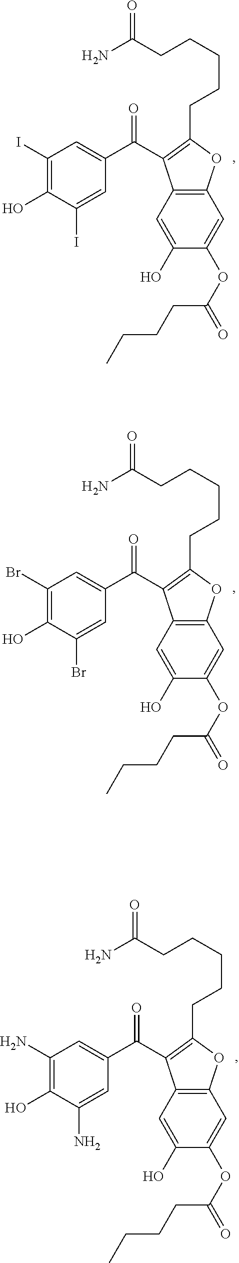 [SMILES: CCCCC(=O)Oc1cc2oc(CCCCCC(N)=O)c(C(=O)c3cc(I)c(O)c(I)c3)c2cc1O ; CCCCC(=O)Oc1cc2oc(CCCCCC(N)=O)c(C(=O)c3cc(Br)c(O)c(Br)c3)c2cc1O ; CCCCC(=O)Oc1cc2oc(CCCCCC(N)=O)c(C(=O)c3cc(N)c(O)c(N)c3)c2cc1O] 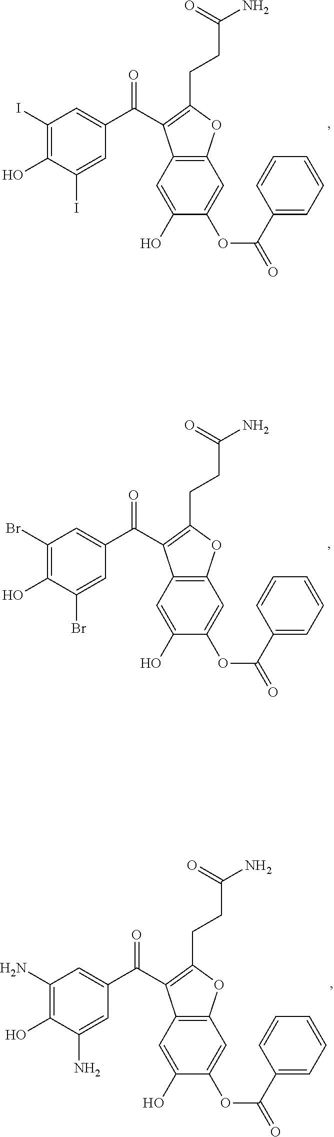 [SMILES: NC(=O)CCc1oc2cc(OC(=O)c3ccccc3)c(O)cc2c1C(=O)c1cc(I)c(O)c(I)c1 ; NC(=O)CCc1oc2cc(OC(=O)c3ccccc3)c(O)cc2c1C(=O)c1cc(Br)c(O)c(Br)c1 ; NC(=O)CCc1oc2cc(OC(=O)c3ccccc3)c(O)cc2c1C(=O)c1cc(N)c(O)c(N)c1] 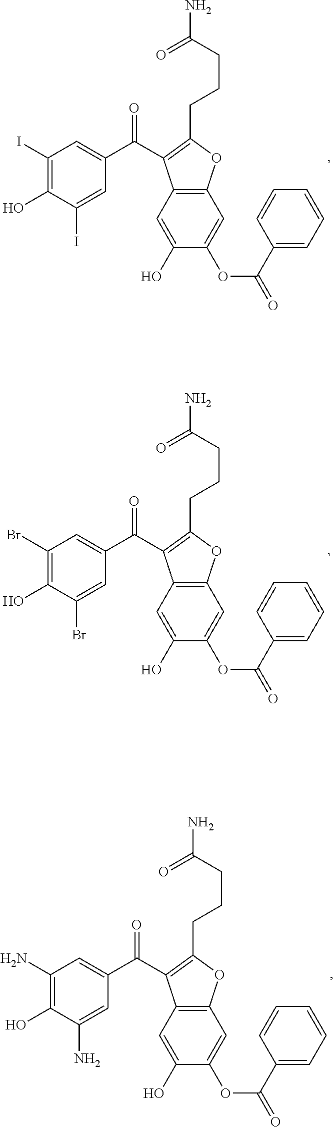 [SMILES: NC(=O)CCCc1oc2cc(OC(=O)c3ccccc3)c(O)cc2c1C(=O)c1cc(I)c(O)c(I)c1 ; NC(=O)CCCc1oc2cc(OC(=O)c3ccccc3)c(O)cc2c1C(=O)c1cc(Br)c(O)c(Br)c1 ; NC(=O)CCCc1oc2cc(OC(=O)c3ccccc3)c(O)cc2c1C(=O)c1cc(N)c(O)c(N)c1] 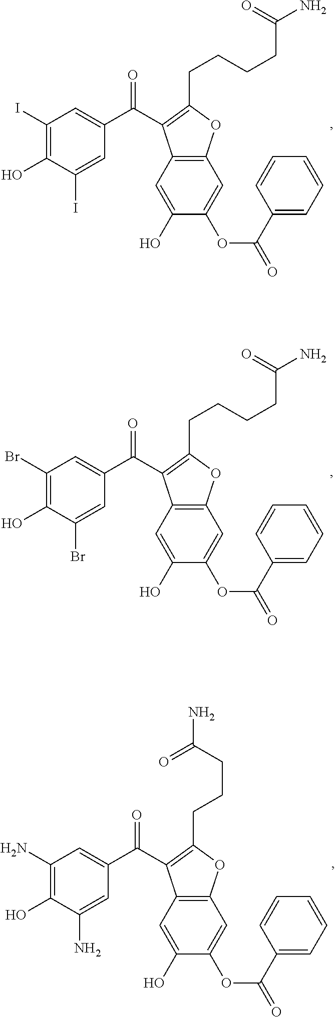 [SMILES: NC(=O)CCCCc1oc2cc(OC(=O)c3ccccc3)c(O)cc2c1C(=O)c1cc(I)c(O)c(I)c1 ; NC(=O)CCCCc1oc2cc(OC(=O)c3ccccc3)c(O)cc2c1C(=O)c1cc(Br)c(O)c(Br)c1 ; NC(=O)CCCc1oc2cc(OC(=O)c3ccccc3)c(O)cc2c1C(=O)c1cc(N)c(O)c(N)c1] 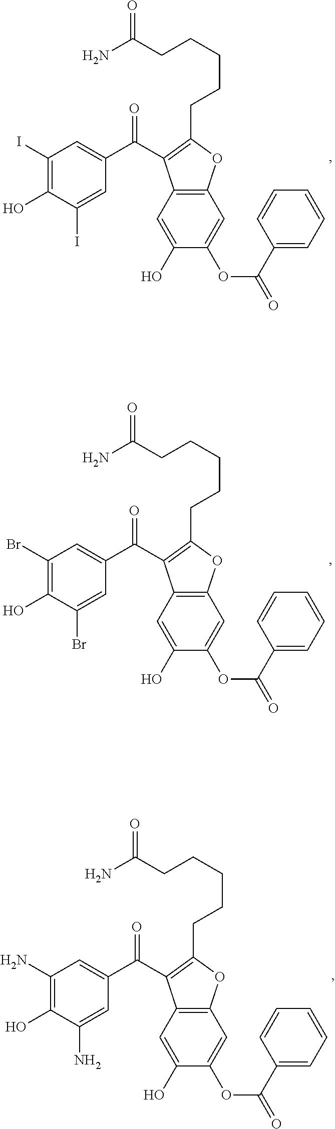 [SMILES: NC(=O)CCCCCc1oc2cc(OC(=O)c3ccccc3)c(O)cc2c1C(=O)c1cc(I)c(O)c(I)c1 ; NC(=O)CCCCCc1oc2cc(OC(=O)c3ccccc3)c(O)cc2c1C(=O)c1cc(Br)c(O)c(Br)c1 ; NC(=O)CCCCCc1oc2cc(OC(=O)c3ccccc3)c(O)cc2c1C(=O)c1cc(N)c(O)c(N)c1] 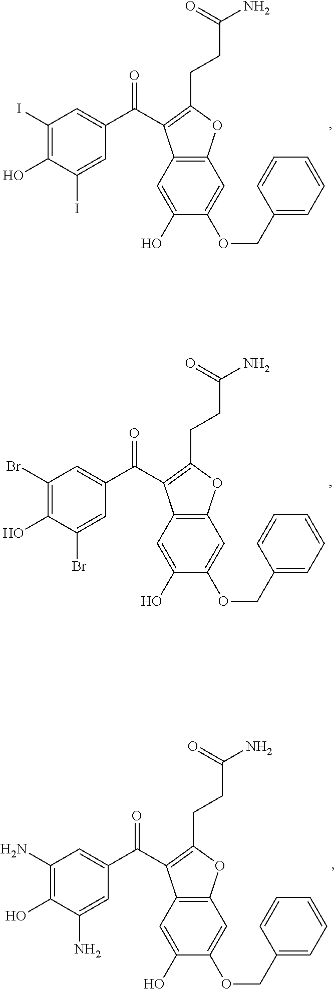 [SMILES: NC(=O)CCc1oc2cc(OCc3ccccc3)c(O)cc2c1C(=O)c1cc(I)c(O)c(I)c1 ; NC(=O)CCc1oc2cc(OCc3ccccc3)c(O)cc2c1C(=O)c1cc(Br)c(O)c(Br)c1 ; NC(=O)CCc1oc2cc(OCc3ccccc3)c(O)cc2c1C(=O)c1cc(N)c(O)c(N)c1] 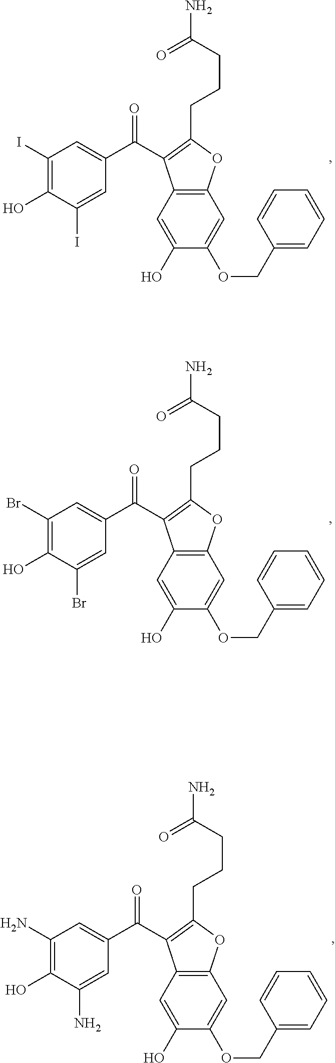 [SMILES: NC(=O)CCCc1oc2cc(OCc3ccccc3)c(O)cc2c1C(=O)c1cc(I)c(O)c(I)c1 ; NC(=O)CCCc1oc2cc(OCc3ccccc3)c(O)cc2c1C(=O)c1cc(Br)c(O)c(Br)c1 ; NC(=O)CCCc1oc2cc(OCc3ccccc3)c(O)cc2c1C(=O)c1cc(N)c(O)c(N)c1] 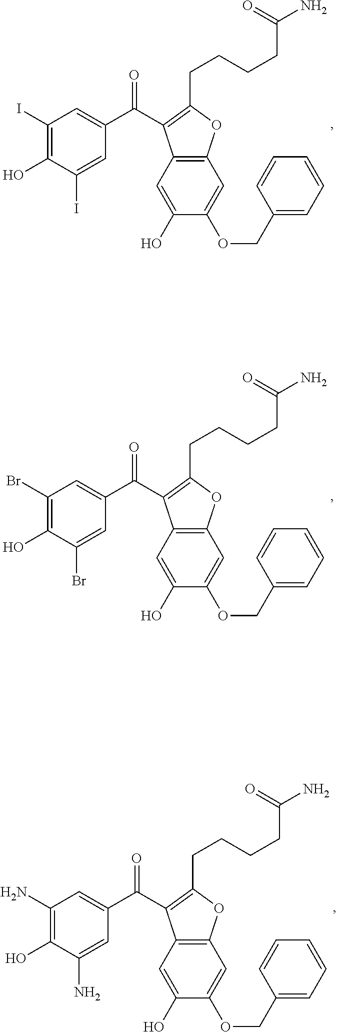 [SMILES: NC(=O)CCCCc1oc2cc(OCc3ccccc3)c(O)cc2c1C(=O)c1cc(I)c(O)c(I)c1 ; NC(=O)CCCCc1oc2cc(OCc3ccccc3)c(O)cc2c1C(=O)c1cc(Br)c(O)c(Br)c1 ; NC(=O)CCCCc1oc2cc(OCc3ccccc3)c(O)cc2c1C(=O)c1cc(N)c(O)c(N)c1] 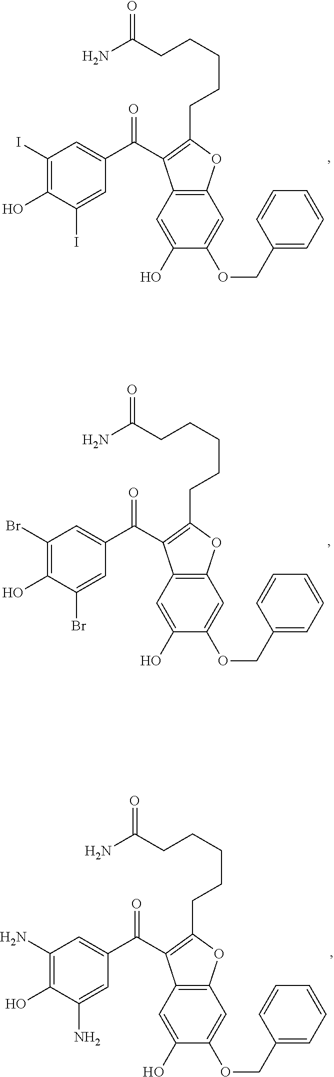 [SMILES: NC(=O)CCCCCc1oc2cc(OCc3ccccc3)c(O)cc2c1C(=O)c1cc(I)c(O)c(I)c1 ; NC(=O)CCCCCc1oc2cc(OCc3ccccc3)c(O)cc2c1C(=O)c1cc(Br)c(O)c(Br)c1 ; NC(=O)CCCCCc1oc2cc(OCc3ccccc3)c(O)cc2c1C(=O)c1cc(N)c(O)c(N)c1] 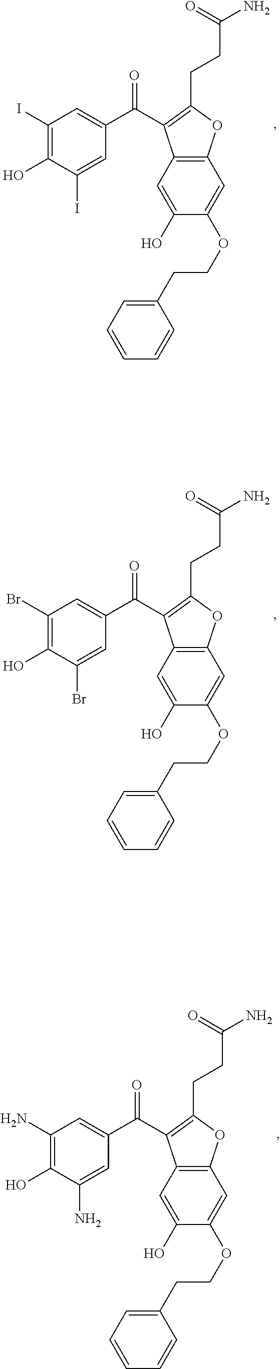 [SMILES: NC(=O)CCc1oc2cc(OCCc3ccccc3)c(O)cc2c1C(=O)c1cc(I)c(O)c(I)c1 ; NC(=O)CCc1oc2cc(OCCc3ccccc3)c(O)cc2c1C(=O)c1cc(Br)c(O)c(Br)c1 ; NC(=O)CCc1oc2cc(OCCc3ccccc3)c(O)cc2c1C(=O)c1cc(N)c(O)c(N)c1] 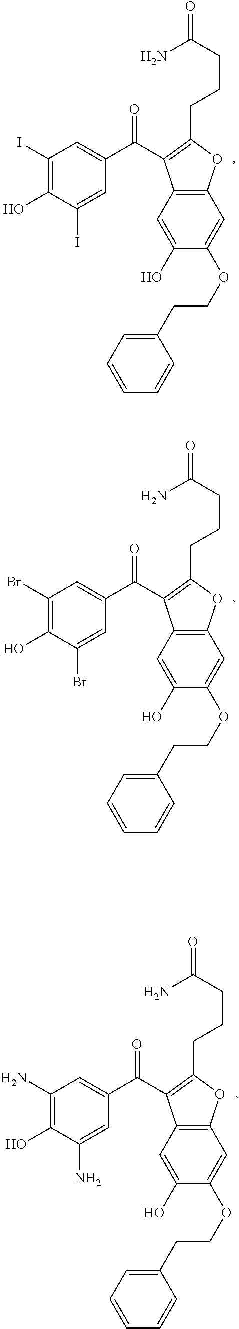 [SMILES: NC(=O)CCCc1oc2cc(OCCc3ccccc3)c(O)cc2c1C(=O)c1cc(I)c(O)c(I)c1 ; NC(=O)CCCc1oc2cc(OCCc3ccccc3)c(O)cc2c1C(=O)c1cc(Br)c(O)c(Br)c1 ; NC(=O)CCCc1oc2cc(OCCc3ccccc3)c(O)cc2c1C(=O)c1cc(N)c(O)c(N)c1] 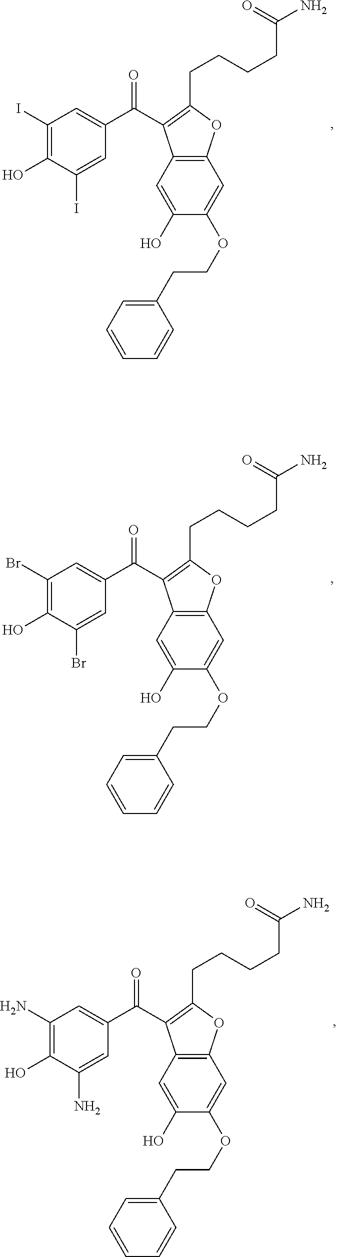 [SMILES: NC(=O)CCCCc1oc2cc(OCCc3ccccc3)c(O)cc2c1C(=O)c1cc(I)c(O)c(I)c1 ; NC(=O)CCCCc1oc2cc(OCCc3ccccc3)c(O)cc2c1C(=O)c1cc(Br)c(O)c(Br)c1 ; NC(=O)CCCCc1oc2cc(OCCc3ccccc3)c(O)cc2c1C(=O)c1cc(N)c(O)c(N)c1] 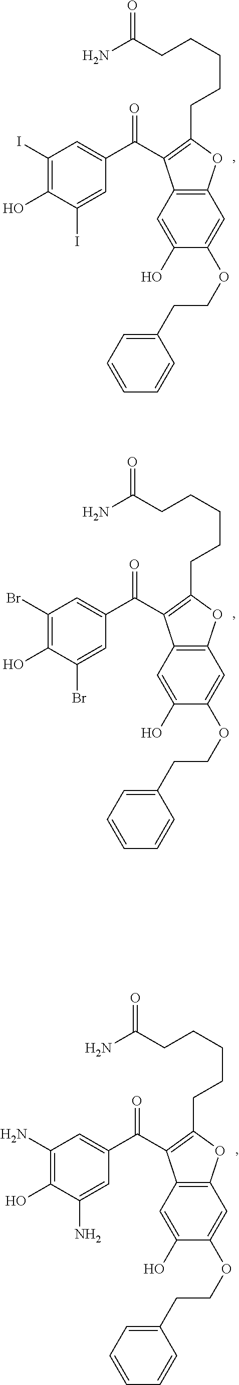 [SMILES: NC(=O)CCCCCc1oc2cc(OCCc3ccccc3)c(O)cc2c1C(=O)c1cc(I)c(O)c(I)c1 ; NC(=O)CCCCCc1oc2cc(OCCc3ccccc3)c(O)cc2c1C(=O)c1cc(Br)c(O)c(Br)c1 ; NC(=O)CCCCCc1oc2cc(OCCc3ccccc3)c(O)cc2c1C(=O)c1cc(N)c(O)c(N)c1] 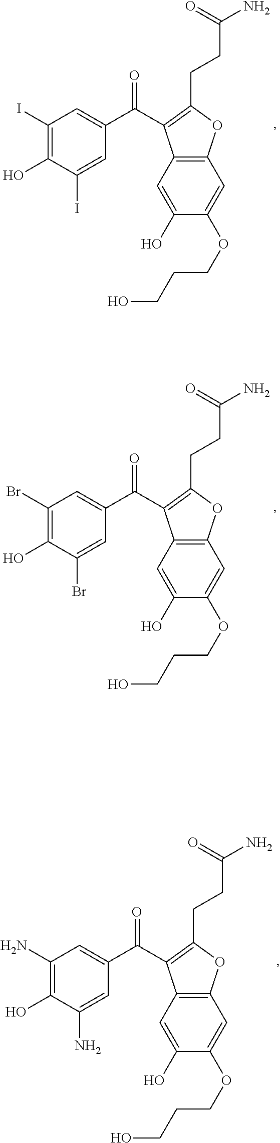 [SMILES: NC(=O)CCc1oc2cc(OCCCO)c(O)cc2c1C(=O)c1cc(I)c(O)c(I)c1 ; NC(=O)CCc1oc2cc(OCCCO)c(O)cc2c1C(=O)c1cc(Br)c(O)c(Br)c1 ; NC(=O)CCc1oc2cc(OCCCO)c(O)cc2c1C(=O)c1cc(N)c(O)c(N)c1] 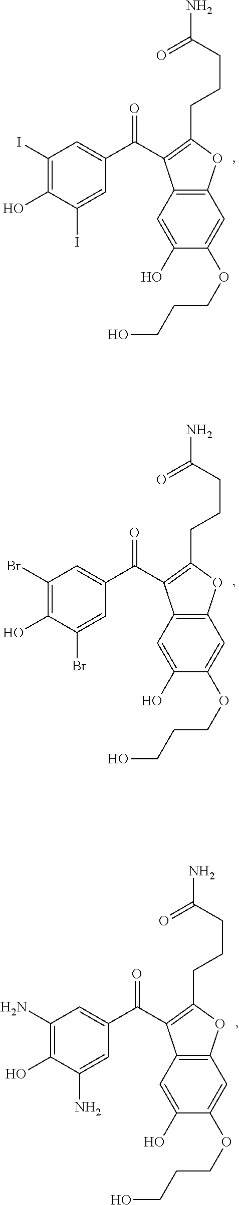 [SMILES: NC(=O)CCCc1oc2cc(OCCCO)c(O)cc2c1C(=O)c1cc(I)c(O)c(I)c1 ; NC(=O)CCCc1oc2cc(OCCCO)c(O)cc2c1C(=O)c1cc(Br)c(O)c(Br)c1 ; NC(=O)CCCc1oc2cc(OCCCO)c(O)cc2c1C(=O)c1cc(N)c(O)c(N)c1] 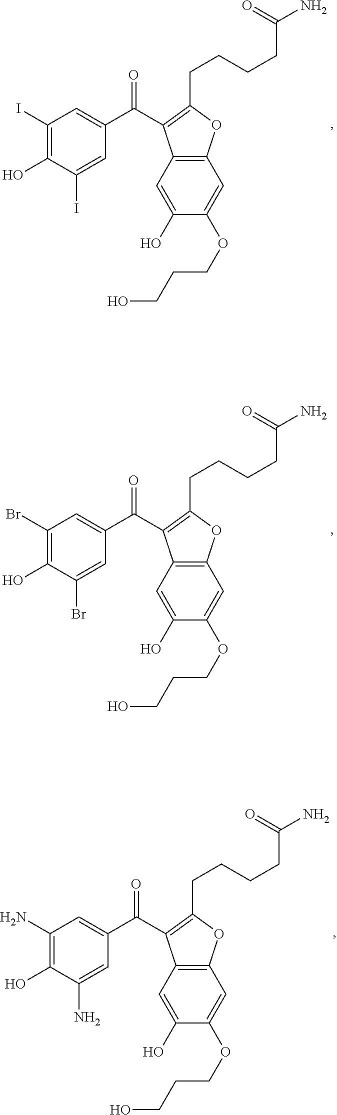 [SMILES: NC(=O)CCCCc1oc2cc(OCCCO)c(O)cc2c1C(=O)c1cc(I)c(O)c(I)c1 ; NC(=O)CCCCc1oc2cc(OCCCO)c(O)cc2c1C(=O)c1cc(Br)c(O)c(Br)c1 ; NC(=O)CCCCc1oc2cc(OCCCO)c(O)cc2c1C(=O)c1cc(N)c(O)c(N)c1] 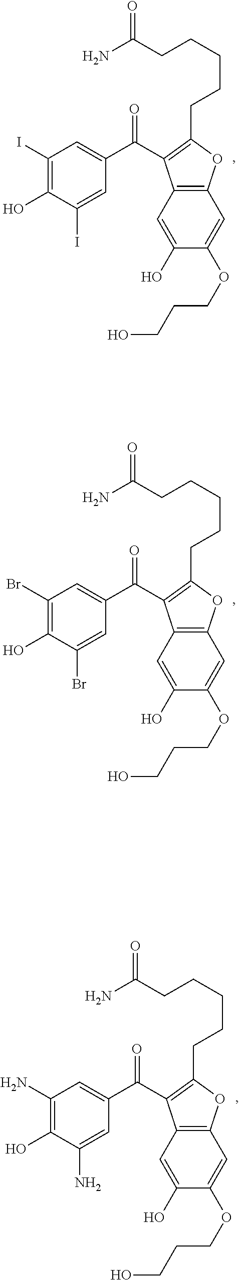 [SMILES: NC(=O)CCCCCc1oc2cc(OCCCO)c(O)cc2c1C(=O)c1cc(I)c(O)c(I)c1 ; NC(=O)CCCCCc1oc2cc(OCCCO)c(O)cc2c1C(=O)c1cc(Br)c(O)c(Br)c1 ; NC(=O)CCCCCc1oc2cc(OCCCO)c(O)cc2c1C(=O)c1cc(N)c(O)c(N)c1] 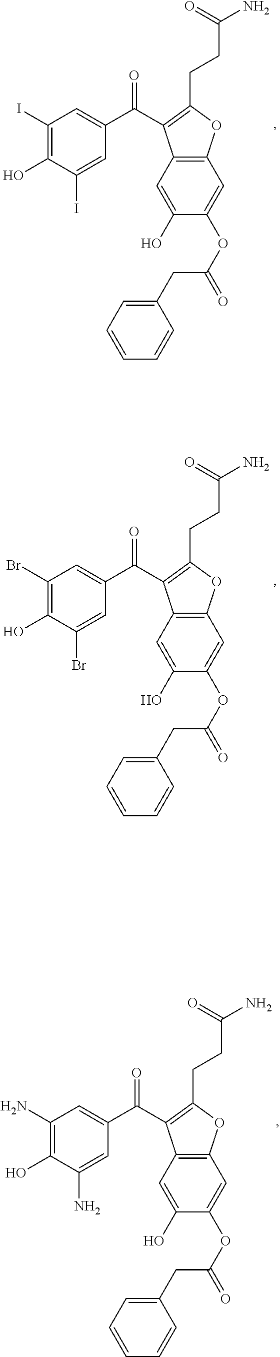 [SMILES: NC(=O)CCc1oc2cc(OC(=O)Cc3ccccc3)c(O)cc2c1C(=O)c1cc(I)c(O)c(I)c1 ; NC(=O)CCc1oc2cc(OC(=O)Cc3ccccc3)c(O)cc2c1C(=O)c1cc(Br)c(O)c(Br)c1 ; NC(=O)CCc1oc2cc(OC(=O)Cc3ccccc3)c(O)cc2c1C(=O)c1cc(N)c(O)c(N)c1] 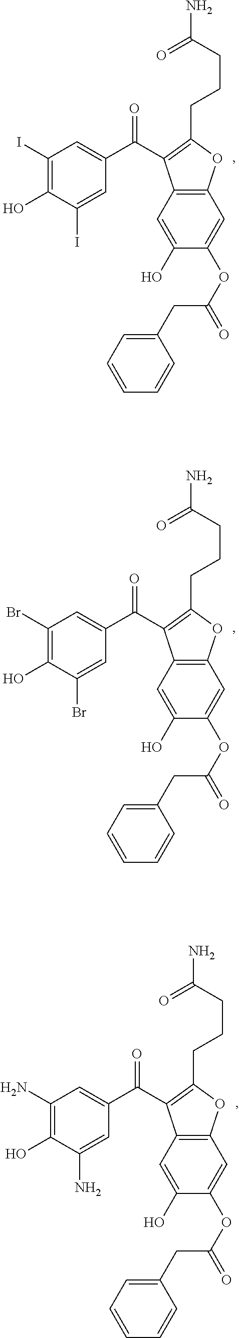 [SMILES: NC(=O)CCCc1oc2cc(OC(=O)Cc3ccccc3)c(O)cc2c1C(=O)c1cc(I)c(O)c(I)c1 ; NC(=O)CCCc1oc2cc(OC(=O)Cc3ccccc3)c(O)cc2c1C(=O)c1cc(Br)c(O)c(Br)c1 ; NC(=O)CCCc1oc2cc(OC(=O)Cc3ccccc3)c(O)cc2c1C(=O)c1cc(N)c(O)c(N)c1] 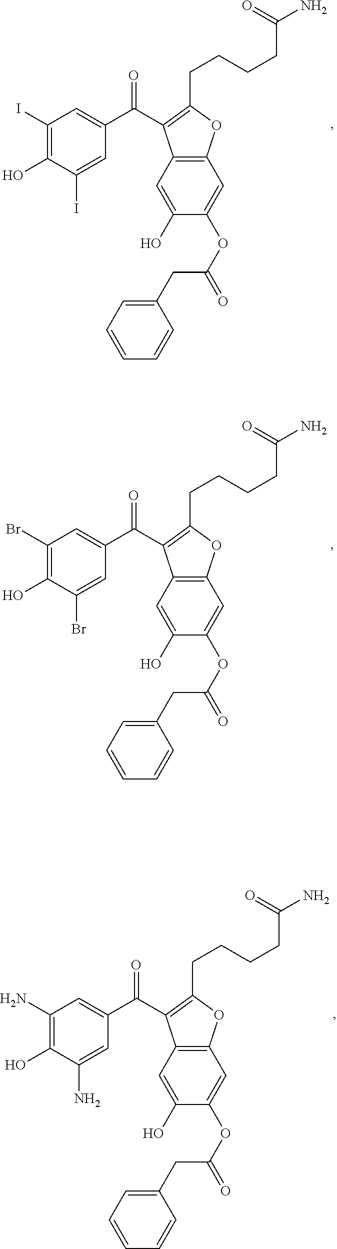 [SMILES: NC(=O)CCCCc1oc2cc(OC(=O)Cc3ccccc3)c(O)cc2c1C(=O)c1cc(I)c(O)c(I)c1 ; NC(=O)CCCCc1oc2cc(OC(=O)Cc3ccccc3)c(O)cc2c1C(=O)c1cc(Br)c(O)c(Br)c1 ; NC(=O)CCCCc1oc2cc(OC(=O)Cc3ccccc3)c(O)cc2c1C(=O)c1cc(N)c(O)c(N)c1] 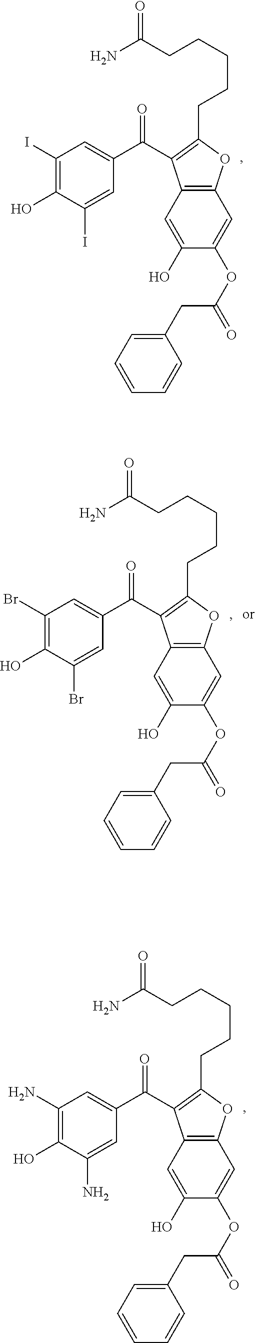 [SMILES: NC(=O)CCCCCc1oc2cc(OC(=O)Cc3ccccc3)c(O)cc2c1C(=O)c1cc(I)c(O)c(I)c1 ; NC(=O)CCCCCc1oc2cc(OC(=O)Cc3ccccc3)c(O)cc2c1C(=O)c1cc(Br)c(O)c(Br)c1 ; NC(=O)CCCCCc1oc2cc(OC(=O)Cc3ccccc3)c(O)cc2c1C(=O)c1cc(N)c(O)c(N)c1]
or a pharmaceutically acceptable salt thereof.

20. The compound of claim 1, wherein the compound is: 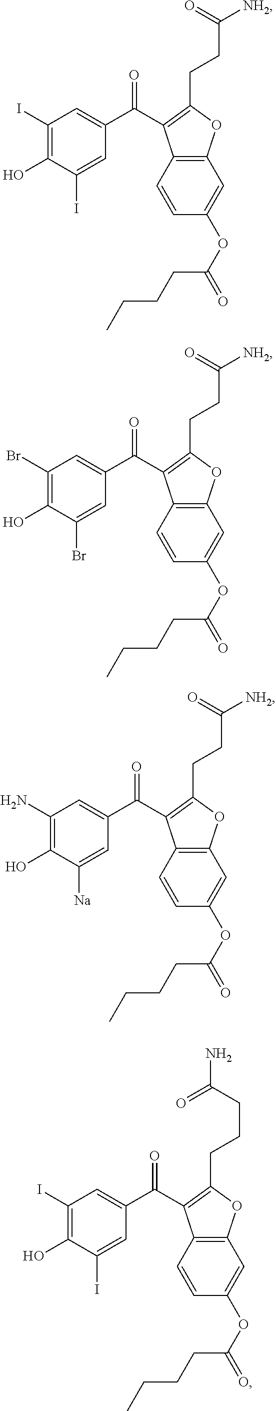 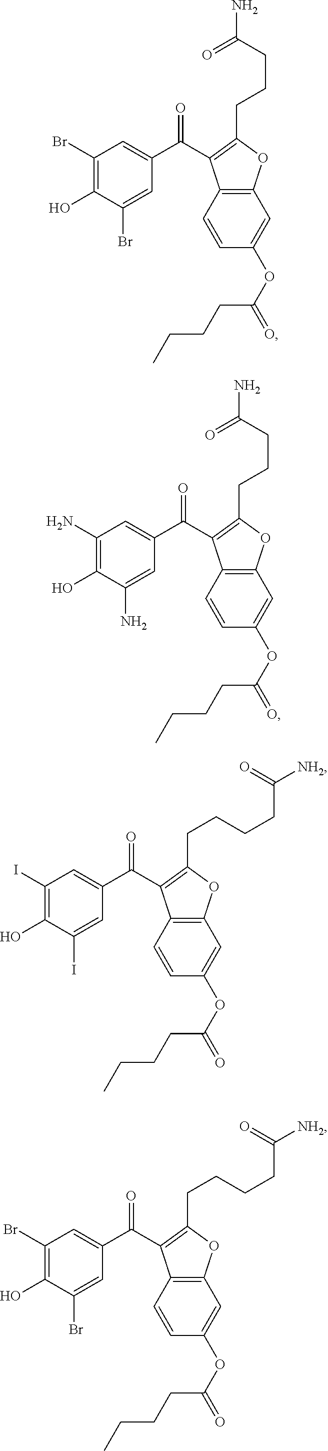 [SMILES: CCCCC(=O)Oc1ccc2c(C(=O)c3cc(Br)c(O)c(Br)c3)c(CCCC(N)=O)oc2c1 ; CCCCC(=O)Oc1ccc2c(C(=O)c3cc(N)c(O)c(N)c3)c(CCCC(N)=O)oc2c1 ; CCCCC(=O)Oc1ccc2c(C(=O)c3cc(I)c(O)c(I)c3)c(CCCCC(N)=O)oc2c1 ; CCCCC(=O)Oc1ccc2c(C(=O)c3cc(Br)c(O)c(Br)c3)c(CCCCC(N)=O)oc2c1] 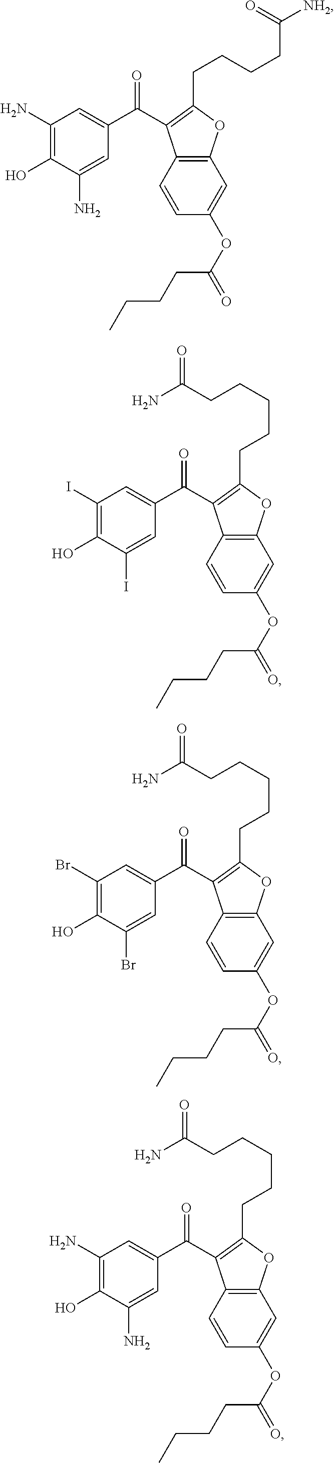 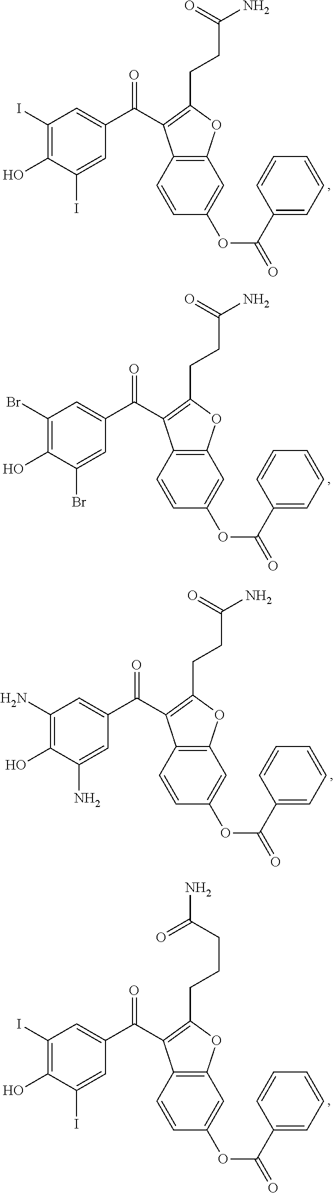 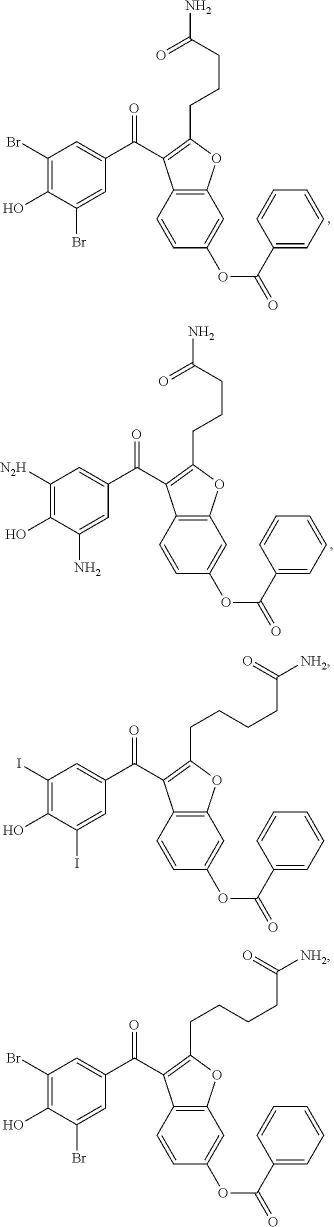 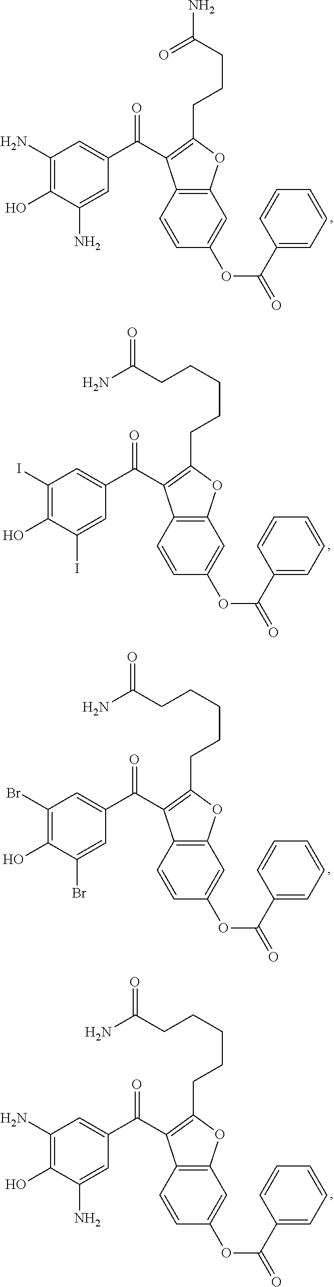 [SMILES: NC(=O)CCCc1oc2cc(OC(=O)c3ccccc3)ccc2c1C(=O)c1cc(N)c(O)c(N)c1 ; NC(=O)CCCCCc1oc2cc(OC(=O)c3ccccc3)ccc2c1C(=O)c1cc(I)c(O)c(I)c1 ; NC(=O)CCCCCc1oc2cc(OC(=O)c3ccccc3)ccc2c1C(=O)c1cc(Br)c(O)c(Br)c1 ; NC(=O)CCCCCc1oc2cc(OC(=O)c3ccccc3)ccc2c1C(=O)c1cc(N)c(O)c(N)c1] 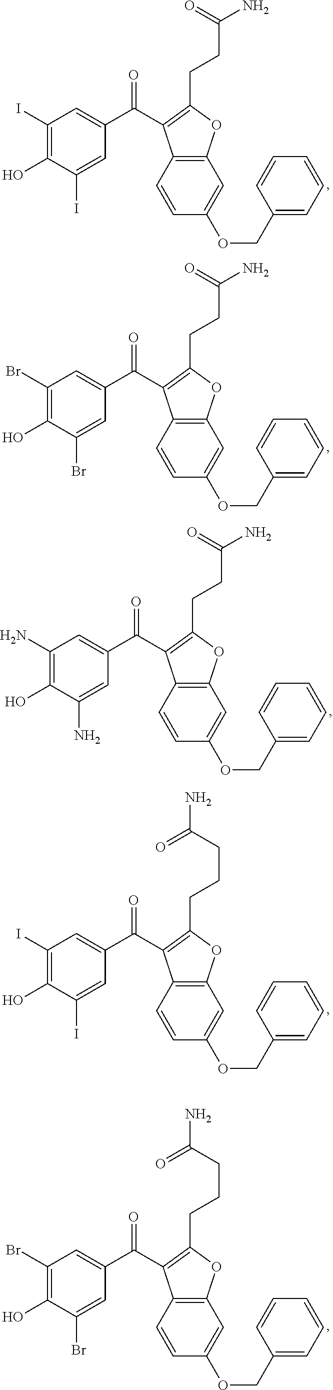 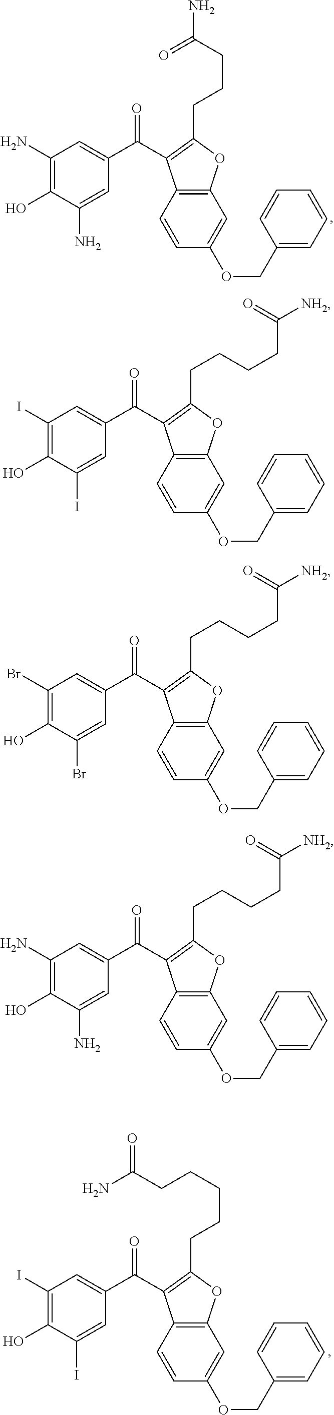 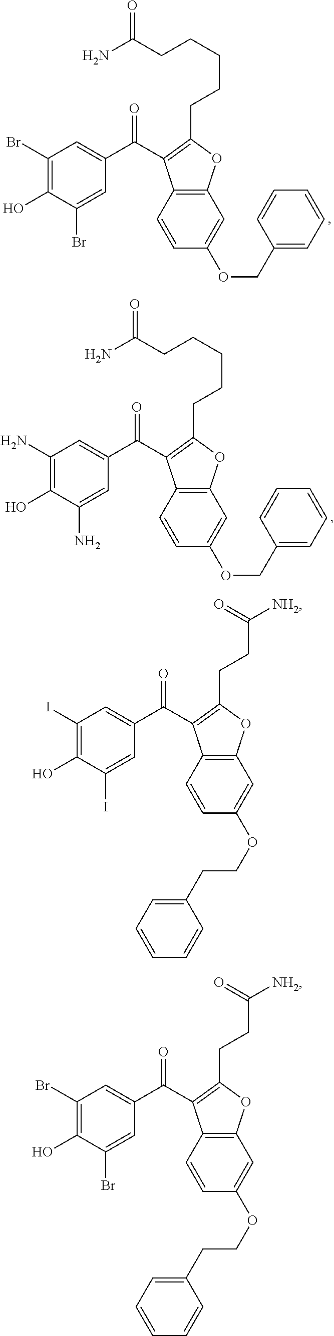 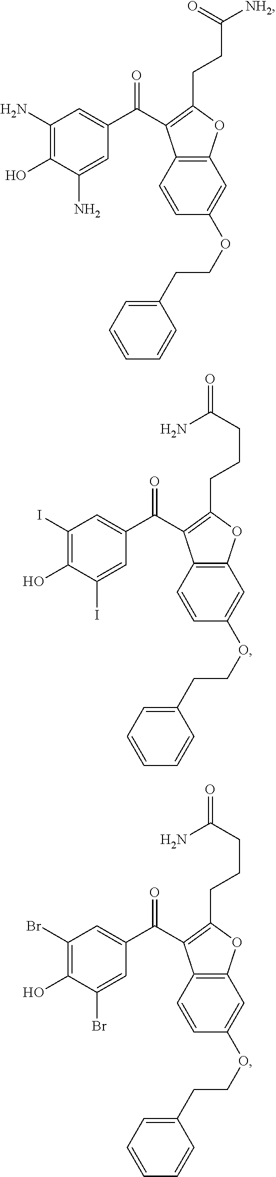 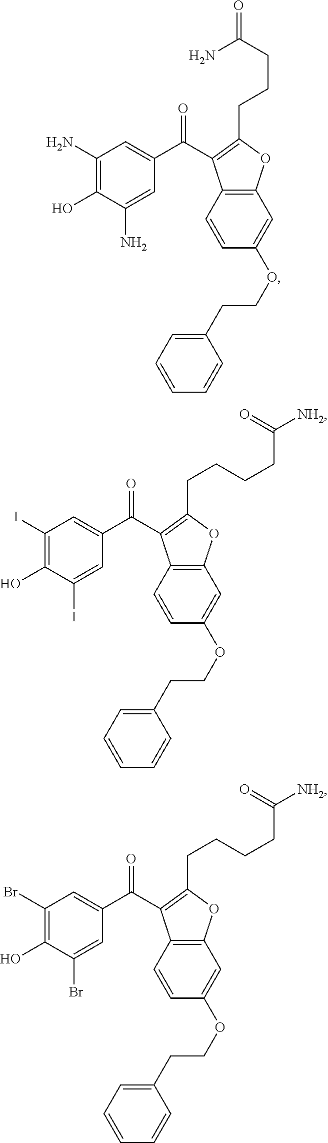 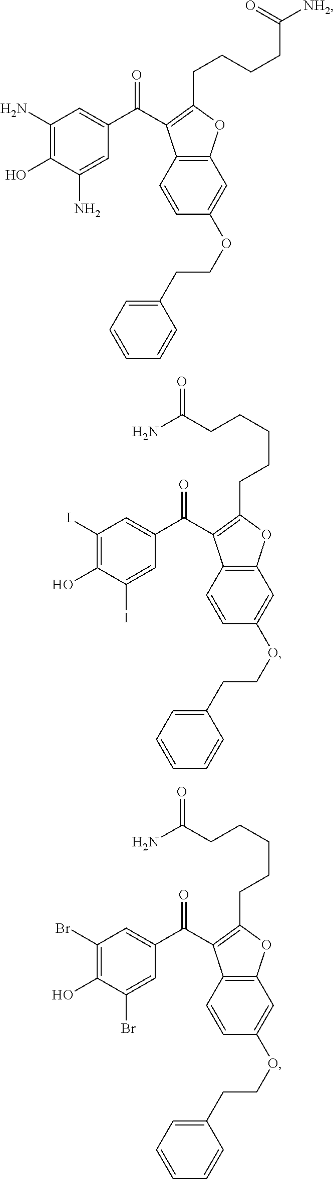 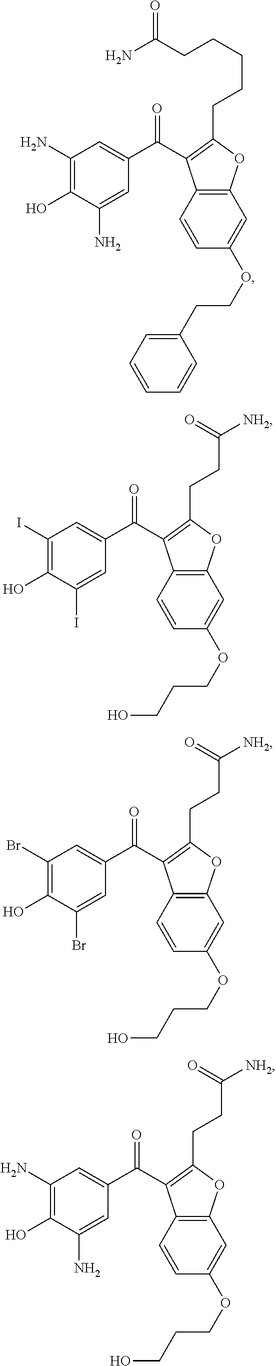 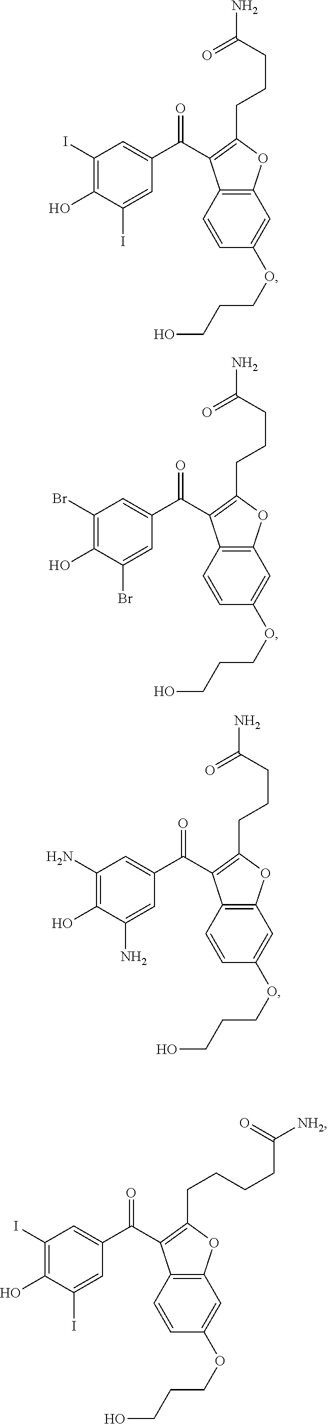 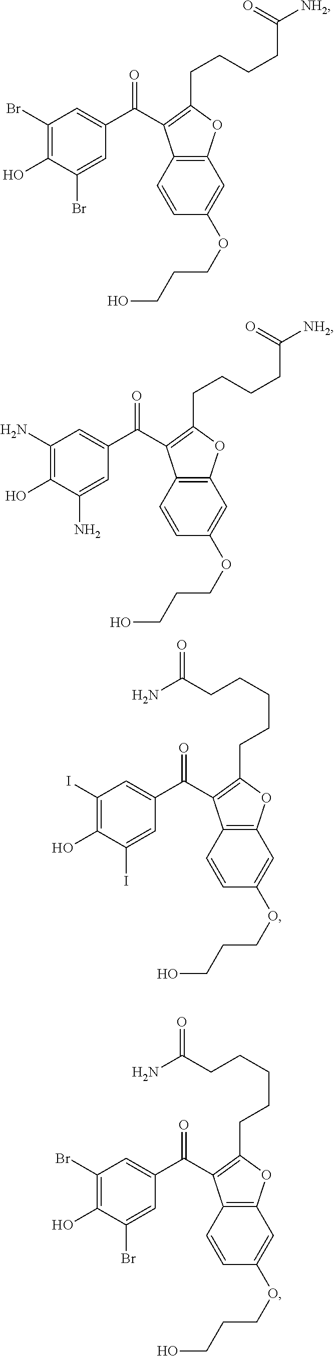 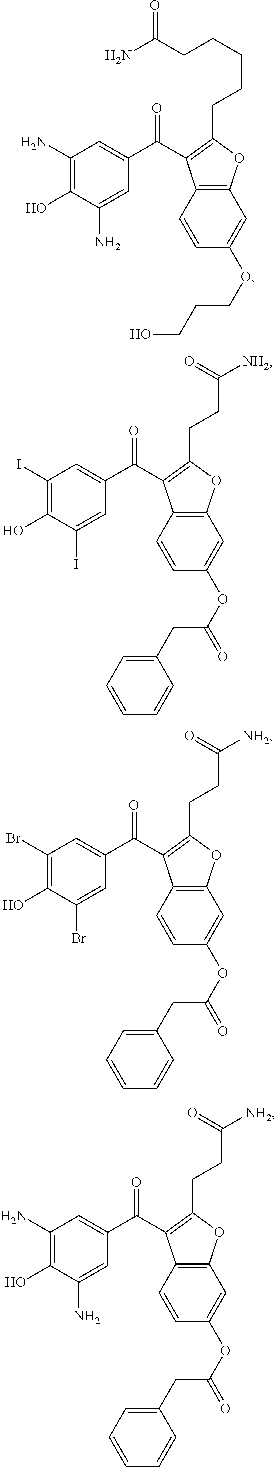 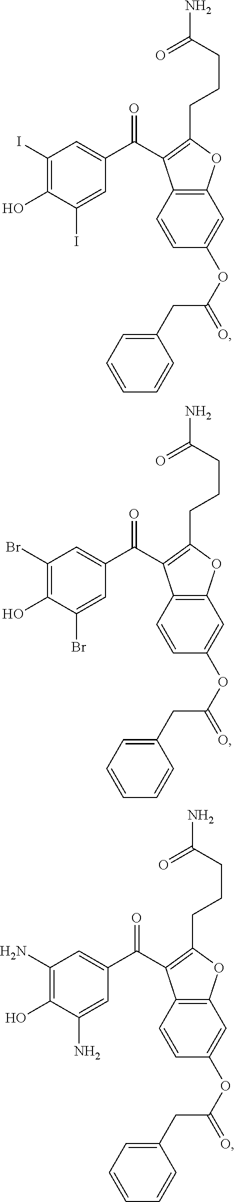 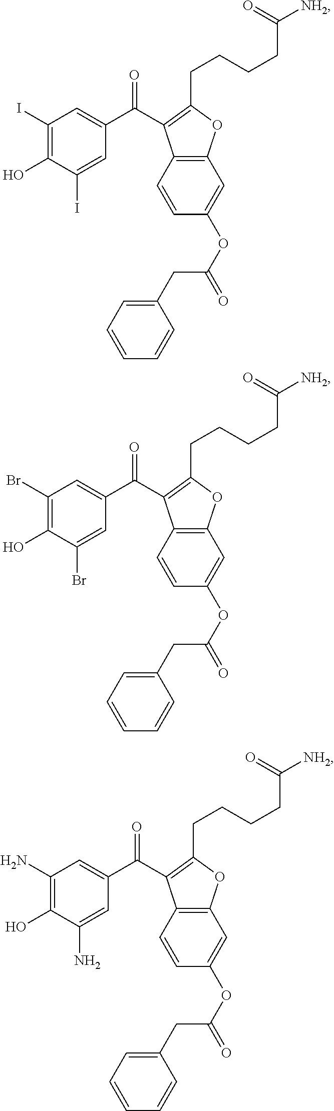 [SMILES: NC(=O)CCCCc1oc2cc(OC(=O)Cc3ccccc3)ccc2c1C(=O)c1cc(I)c(O)c(I)c1 ; NC(=O)CCCCc1oc2cc(OC(=O)Cc3ccccc3)ccc2c1C(=O)c1cc(Br)c(O)c(Br)c1 ; NC(=O)CCCCc1oc2cc(OC(=O)Cc3ccccc3)ccc2c1C(=O)c1cc(N)c(O)c(N)c1] 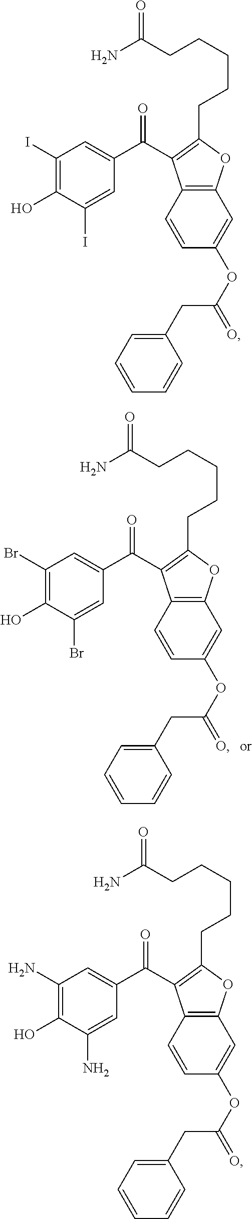
or a pharmaceutically acceptable salt thereof.

21. (canceled)

22. A method of treating a disease in an individual, comprising:
selecting or identifying an individual having pulmonary arterial hypertension;
administering to the individual an effective amount of a compound of claim 1.

23. A method of treating a disease in an individual, comprising:
selecting or identifying an individual having angio-obliterative pulmonary hypertension, or idiopathic pulmonary arterial hypertension;
administering to the individual an effective amount of a compound of claim 1.

24. (canceled)

25. (canceled)

26. A method of treating a disease in an individual, comprising:
selecting or identifying an individual having pulmonary arterial hypertension;
administering to the individual an effective amount of a compound having the structure of Formula IV: 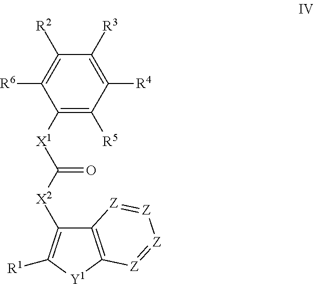
or a pharmaceutically acceptable salt thereof,
wherein:
R¹ is selected from the group consisting of C₁₋₆alkyl, C₂₋₆alkenyl, C₂₋₆alkynyl, aryl, heteroaryl, heterocyclyl, arylalkyl, heteroarylalkyl, heterocyclylalkyl, cycloalkyl, cycloalkenyl, (cyclolalkyl)alkyl, and amino, said C₁₋₆ alkyl, C₂₋₆alkenyl, C₂₋₆alkynyl, aryl, heteroaryl, heterocyclyl, arylalkyl, heteroarylalkyl, heterocyclylalkyl, cycloalkyl, cycloalkenyl, and (cyclolalkyl)alkyl are each optionally substituted with one or more R^{1A};
each R^{1A} is independently selected from the group consisting of hydroxy, halo, cyano, nitro, C₁₋₆ alkyl optionally substituted with up to 5 fluoro, C₁₋₆alkoxy optionally substituted with up to 5 fluoro;
R² is selected from the group consisting of halo, hydroxy, and C₁₋₆ alkyl substituted with one or more hydroxy;
R³ is selected from the group consisting of halo, hydroxy, and C₁₋₆ alkyl substituted with one or more hydroxy;
R⁴ is halo;
R⁵ and R⁶ are each independently selected from the group consisting of H (hydrogen), halo, cyano, C₁₋₆ alkyl, aryl, heteroaryl, heterocyclyl, and amino, said C₁₋₆ alkyl, aryl, heteroaryl, and heterocyclyl each optionally substituted with one or more R^{1A};
X¹ is absent;
X² is absent;
Y is O (oxygen);
each Z is CR^{2A}; and
each R^{2A} is independently selected from the group consisting of H (hydrogen), halo, hydroxy, O-carbamyl, N-carbamyl, C-amido, S-sulfonamido, N-sulfonamido, C-carboxy, amino, aryl, heteroaryl, heterocyclyl, arylalkyl, heteroarylalkyl, heterocyclylalkyl, cycloalkyl, cycloalkenyl, (cyclolalkyl)alkyl, C₁₋₆ alkyl substituted with one or more hydroxyl, and C₁₋₆ alkyl optionally substituted with up to 5 fluoro.

27. (canceled)

28. The method of claim 26, wherein the compound of Formula IV has the structure of Formula V: 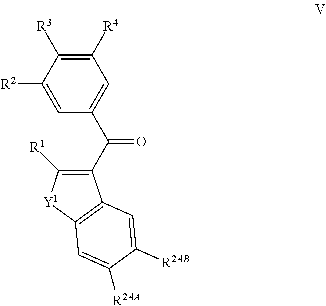
or a pharmaceutically acceptable salt thereof,
wherein:
R³ is halo or hydroxy;
R^{2AA} is H (hydrogen) or hydroxyl; and
R^{2AB} is H (hydrogen) or hydroxyl.

29. (canceled)

30. (canceled)

31. (canceled)

32. (canceled)

33. (canceled)

34. (canceled)

35. (canceled)

36. (canceled)
